(19) United States
(12) Patent Application Publication
(10) Pub. No.: US 2025/0051471 A1
(43) Pub. Date: Feb. 13, 2025

(54) Anti-Abcb1 Antibodies

(71) Applicant: WILLIAM ROBERT ARATHOON LIVING TRUST DATED AUGUST 29, 2016, Tiburon, CA (US)

(72) Inventors: William Robert Arathoon, Tiburon, CA (US); Mastewal Abuhay, Tiburon, CA (US); Raffaella Briante, Tiburon, CA (US); Alissa Loren O'Connor, Tiburon, CA (US); Paul David Ponath, Tiburon, CA (US); Cindy Tan, Tiburon, CA (US); Qianting Zhai, Tiburon, CA (US); Pingping Zhang, Tiburon, CA (US)

(21) Appl. No.: 18/716,407

(22) PCT Filed: Dec. 5, 2022
(86) PCT No.: PCT/US22/80932

§ 371 Date: Jun. 4, 2024

Related U.S. Application Data
(60) Provisional application No. 63/289,007, filed on Dec. 13, 2021.
(60) Provisional application No. 63/335,491, filed on Apr. 27, 2022.

(51) Int. Cl. C07K 16/28; A61K 39/00; A61K 45/06; A61P 35/00

(52) U.S. Cl. CPC C07K 16/2896; A61K 45/06; A61P 35/00; C07K 16/2803; A61K 2039/505; C07K 2317/24; C07K 2317/31; C07K 2317/52; C07K 2317/565

(57) ABSTRACT

Provided are anti-ABCB 1 antibodies that may be used as multi-specific, including bispecific, antibodies targeting both ABCB 1 and a tumor-associated antigen (TAA), as well as pharmaceutical compositions, nucleic acids, recombinant expression vectors, cells, and kits that include or encode such antibodies and multi-specific antibodies. Further included are uses and methods of treating conditions where inhibition of ABCB 1-mediated efflux transport is desirable, such as, cancer or multi-drug resistance mediated by the ABCB1 transporter, e.g., multi-drug resistant cancer. In particular, the disclosure provides humanized multi-specific, e.g. bispecific, IgG (IgG1) antibodies that bind ABCB1 and a TAA. Multi-specific IgG1 antibody molecules are provided with Fc substitutions in at least one of a first and a second heavy chain constant region.

### CROSS-REFERENCE TO RELATED APPLICATIONS

This application claims the benefit of priority to U.S. Provisional Patent Application No. 63/289,007 filed on Dec. 13, 2021 and U.S. Provisional Patent Application No. 63/335,491 filed on Apr. 27, 2022, which application are incorporated herein by reference in their entireties.

### INCORPORATION BY REFERENCE OF MATERIAL ELECTRONICALLY SUBMITTED

A Sequence Listing is provided herewith as a Sequence Listing XML, “KNJY-008WO_SEQ_LIST,” created on Oct. 28, 2022 and having a size of 121,000 bytes. The contents of the text file are incorporated by reference herein in their entirety.

### INTRODUCTION

Drug resistance, a well-known phenomenon that results when diseases become tolerant to pharmaceutical treatments, is a major and increasing challenge in various fields of medicine, including oncology. Some methods of drug resistance are disease-specific, while others, such as drug efflux, which is observed in microbes and human drug-resistant cancers, are evolutionarily conserved. Although many types of cancers are initially susceptible to chemotherapy, over time they can develop resistance through these and other mechanisms, such as DNA mutations and metabolic changes that promote drug inhibition, degradation, and enhanced efflux.

Efflux pumps (EP) are proteins expressed by virtually all living cells and have evolved to naturally expel various compounds from the cells. Members of the ATP-binding cassette (ABC) transporter family proteins are examples of EPs that enable drug efflux and are important, well-studied regulators at the plasma membranes of healthy cells. Though a transporter's structure varies from protein to protein (e.g., there are 49 known members of the ABC family in humans), they are all classified by the presence of two distinct domains—a highly conserved nucleotide binding domain and a more variable transmembrane domain. Multidrug resistance protein 1, encoded by the ATP Binding Cassette Subfamily B Member 1 (MDR1, ABCB1, P-glycoprotein) gene, was the first of these to be identified and has been studied extensively. Normal expression of ABCB1 is increased in certain tissues (e.g., colon, liver, and kidney) when these tissues become neoplastic and increased expression in response to treatment with certain chemotherapeutics demonstrates that both intrinsic and extrinsic mechanisms of ABCB1 overexpression are at play.

EPs enable cells and tumors to develop resistance to chemotherapeutic agents. Such resistance is frequently associated with enhanced efflux of the treatment molecules from the resistant cells. This chemotherapy resistance is termed multi drug resistance (MDR) when it applies to more than one chemotherapeutic agent. Various small molecule inhibitors have been developed that target and inhibit EPs but none have been successful in the human clinical setting for a variety of reasons among which is their tendency to penetrate into and affect all cells in the body, including healthy cells that employ EPs for efflux of naturally occurring cellular toxins, regardless of the function of the cells or their efflux pumps.

Among cancer patients, where metastatic cancer cell populations have largely been killed and cleared from the patients by use of chemotherapy, it is not uncommon for a drug-resistant cancerous population of cells to emerge and spread without response to renewed treatment with the earlier therapy. In most cases, a different drug with a different mechanism of action is applied until, once again, another emergent population of drug-resistant cells and/or tumor develops.

Multi-specific antibodies binding to ABCB1 and a tumor-associated antigen are disclosed in WO2020206033 the entire disclosure of which is hereby expressly incorporated by reference.

### SUMMARY

Provided are anti-ABCB1 antibodies that may be used as multi-specific, including bispecific, antibodies targeting both ABCB1 and a tumor-associated antigen (TAA), as well as pharmaceutical compositions, nucleic acids, recombinant expression vectors, cells, and kits that include or encode such antibodies and multi-specific antibodies along with methods for making such antibodies. Further included are uses and methods of treating conditions where inhibition of ABCB1-mediated efflux transport is desirable, such as, cancer or multi-drug resistance mediated by the ABCB1 transporter, e.g., multi-drug resistant cancer. In particular, the disclosure provides humanized multi-specific, e.g., bispecific, IgG (IgG1) antibodies that bind ABCB1 and a TAA. Illustrative structures of various bispecific antibody formats within the scope herein are shown in FIGS. 1, 2A and 2B, wherein Target 1 is ABCB1 and Target 2 is a TAA.

In one aspect, multi-specific IgG1 antibody molecules are provided with Fc substitutions in at least one of a first and a second heavy chain constant region.

In one embodiment, such multi-specific IgG1 antibody molecules comprise a first heavy chain that comprises an antigen-binding site for ABCB1 and a second heavy chain that comprises an antigen-binding site for a TAA each heavy chain comprising an Fc region, wherein one of the Fc regions comprises hole amino acid substitutions Y349C, T366S, L368A, and Y407V, in combination with amino acid substitution F405V or Q347R, wherein numbering is according to the EU numbering scheme.

In another embodiment, in such multi-specific IgG1 antibody molecules the other Fc region comprises knob amino acid substitutions S354C and T366W, wherein numbering is according to the EU numbering scheme.

In yet another embodiment, one of the Fc regions further comprises one or both of amino acid substitutions D399K and E356K, wherein numbering is according to the EU numbering scheme.

In a further embodiment, the other Fc region further comprises one or both of amino acid substitutions K409D and K392D, wherein numbering is according to the EU numbering scheme.

Some of the multi-specific, including bispecific IgG1 antibody molecules herein further comprise two identical light variable regions, each comprising an antigen-binding site for ABCB1, wherein the second heavy chain binds TAA when paired with one of the light chain variable regions.

The light chain variable regions may be humanized. In such humanized light chains, the antigen-binding site of the two identical light chain variable regions may comprise light chain CDRs 1-3 (LCDRs 1-3) of a light chain variable region sequence selected from the group consisting of:

(SEQ ID NO: 1)
DIVMTQTPLSSPVTLGQPASISCRSSQSIVHSTGNTYLEWYQQRP
GQPPRLLIYKISNRFSGVPDRFSGSGAGTDFTLKISRVEAEDVGV
YYCFQASHFPRTFGGGTKLEIKR
(MRK16 hmz);
(SEQ ID NO: 2)
DIVMTQSPLSLPVTPGEPASISCRSSQSLVHSNGNTYLEWYLQKP
GQSPQLLIYKVSNRFSGVPDRFSGSGSGTDFTLKISRVEAEDVGV
YYCFQGSHFPRTFGQGTKLEIK
(B1VL6/CDR3v2a hmz);
(SEQ ID NO: 3)
DIVMTQTPLSSPVTLGQPASISCRSSQSIVHSTGNTYLEWYQQKP
GQPPRLLIYKISNRFSGVPDRFSGSGSGTDFTLKISRVEAEDVGV
YYCFQASHFPRTFGQGTKLEIK
(MRK16v4a1 hmz);
(SEQ ID NO: 4)
DIVMTQTPLSSPVTLGQPASISCRSSQSIVHSTGNTYLEWYQQKP
GQPPRLLIYKVSNRFSGVPDRFSGSGSGTDFTLKISRVEAEDVGV
YYCFQASHFPRTFGQGTKLEIK
(MRK16v4a12 hmz);
(SEQ ID NO: 6)
DIVMTQSPLSLPVTPGEPASISCRSSQTIVHSNGNTYLEWYLQKP
GQSPQLLIYKVSKRFSGVPDRFSGSGSGTDFTLKISRVEAEDVGV
YYCFQASHFPRTFGQGTKLEIK
(B1.89v1.huL1 (KV2) hmz);
(SEQ ID NO: 7)
DVVLTQSPLSLPVTPGEPASISCRSSQNIVHSTGNTYLDWYLQKP
GQSPQLLIYKVSNRFSGVPDRFSGSGSGTDFTLKISRVEAEDVGV
YYCFQGSHIPRTFGQGTKLEIKR
(SEQ ID NO: 7) (B2.28.huL2);
and
(SEQ ID NO: 8)
DIVMTQSPLSLPVSLGDPASISCRSSQSLVHSNGNTYLEYYLQKP
GQSPKLLIYKVSNRFSGVPDRFSGSGSGTDFTLKISRVEAEDLGV
YYCFQGSHFPRTFGGGTKLEIK
(B1VL6/CDR3v2 hmz),

wherein LCDRs 1-3 are shown in bold.

In another embodiment, the two identical light chain variable region sequences comprise a sequence selected from the group consisting of:

(SEQ ID NO: 1)
DIVMTQTPLSSPVTLGQPASISCRSSQSIVHSTGNTYLEWYQQRP
GQPPRLLIYKISNRFSGVPDRFSGSGAGTDFTLKISRVEAEDVGV
YYCFQASHFPRTFGGGTKLEIKR
(MRK16 hmz);
(SEQ ID NO: 2)
DIVMTQSPLSLPVTPGEPASISCRSSQSLVHSNGNTYLEWYLQKP
GQSPQLLIYKVSNRFSGVPDRFSGSGSGTDFTLKISRVEAEDVGV
YYCFQGSHFPRTFGQGTKLEIK
(B1VL6/CDR3v2a hmz);
(SEQ ID NO: 3)
DIVMTQTPLSSPVTLGQPASISCRSSQSIVHSTGNTYLEWYQQKP
GQPPRLLIYKISNRFSGVPDRFSGSGSGTDFTLKISRVEAEDVGV
YYCFQASHFPRTFGQGTKLEIK
(MRK16v4a1 hmz);
(SEQ ID NO: 4)
DIVMTQTPLSSPVTLGQPASISCRSSQSIVHSTGNTYLEWYQQKP
GQPPRLLIYKVSNRFSGVPDRFSGSGSGTDFTLKISRVEAEDVGV
YYCFQASHFPRTFGQGTKLEIK
(MRK16v4a12 hmz);
(SEQ ID NO: 6)
DIVMTQSPLSLPVTPGEPASISCRSSQTIVHSNGNTYLEWYLQKP
GQSPQLLIYKVSKRFSGVPDRFSGSGSGTDFTLKISRVEAEDVGV
YYCFQASHFPRTFGQGTKLEIK
(B1.89v1.huL1 (KV2) hmz);
(SEQ ID NO: 7)
DVVLTQSPLSLPVTPGEPASISCRSSQNIVHSTGNTYLDWYLQKP
GQSPQLLIYKVSNRFSGVPDRFSGSGSGTDFTLKISRVEAEDVGV
YYCFQGSHIPRTFGQGTKLEIKR
(B2.28.huL2);
and
(SEQ ID NO: 8)
DIVMTQSPLSLPVSLGDPASISCRSSQSLVHSNGNTYLEYYLQKP
GQSPKLLIYKVSNRFSGVPDRFSGSGSGTDFTLKISRVEAEDLGV
YYCFQGSHFPRTFGGGTKLEIK
(B1VL6/CDR3v2 hmz).

Specifically included herein multi-specific IgG1 antibody molecules, wherein the antigen-binding site of the two identical light chain variable regions comprises light chain CDRs 1-3 (LCDRs 1-3) of a light chain variable region sequence selected from the group consisting of:

(SEQ ID NO: 6)
DIVMTQSPLSLPVTPGEPASISCRSSQTIVHSNGNTYLEWYLQKP
GQSPQLLIYKVSKRFSGVPDRFSGSGSGTDFTLKISRVEAEDVGV
YYCFQASHFPRTFGQGTKLEIK
(B1.89v1.huL1 (KV2) hmz);
(SEQ ID NO: 7)
DVVLTQSPLSLPVTPGEPASISCRSSQNIVHSTGNTYLDWYLQKP
GQSPQLLIYKVSNRFSGVPDRFSGSGSGTDFTLKISRVEAEDVGV
YYCFQGSHIPRTFGQGTKLEIKR
(B2.28.huL2);
and
(SEQ ID NO: 8)
DIVMTQSPLSLPVSLGDPASISCRSSQSLVHSNGNTYLEYYLQKP
GQSPKLLIYKVSNRFSGVPDRFSGSGSGTDFTLKISRVEAEDLGV
YYCFQGSHFPRTFGGGTKLEIK

(B1VL6/CDR3v2 hmz), wherein LCDRs 1-3 are shown in bold, or wherein the two identical light chain variable region sequences comprise the sequence of:

(SEQ ID NO: 6)
DIVMTQSPLSLPVTPGEPASISCRSSQTIVHSNGNTYLEWYLQKP
GQSPQLLIYKVSKRFSGVPDRFSGSGSGTDFTLKISRVEAEDVGV
YYCFQASHFPRTFGQGTKLEIK
(B1.89v1.huL1 (KV2) hmz);
(SEQ ID NO: 7)
DVVLTQSPLSLPVTPGEPASISCRSSQNIVHSTGNTYLDWYLQKP
GQSPQLLIYKVSNRFSGVPDRFSGSGSGTDFTLKISRVEAEDVGV
YYCFQGSHIPRTFGQGTKLEIKR
(B2.28.huL2);
or
(SEQ ID NO: 8)
DIVMTQSPLSLPVSLGDPASISCRSSQSLVHSNGNTYLEYYLQKP
GQSPKLLIYKVSNRFSGVPDRFSGSGSGTDFTLKISRVEAEDLGV
YYCFQGSHFPRTFGGGTKLEIK
(B1VL6/CDR3v2 hmz).

In addition, in multi-specific IgG1 antibody molecules herein the first and/or second heavy chain may be humanized. For example, the antigen-binding site of the first heavy chain variable region may comprise heavy chain CDRs 1-3 (H1CDRs 1-3) of a heavy chain variable region sequence selected from the group consisting of:

(SEQ ID NO: 9)
EVQLVESGGVVVQPGGSLRLSCAASGFTFSRYTMSWVRQAPGKGL
EWVATISSGGGNTYYPDSVKGRFTVSRDNSKNSLYLQMNSLRTED
TALYYCARYGAGDAWFAYWGQGTLVTVSS
(15D3 hmz);
and
(SEQ ID NO: 10)
EVQLVESGGVVVQPGGSLRLSCAASGFTFSRYTMSWVRQAPGKGL
EWVATISSGGGNTYYPDSVKGRFTVSRDNSKNSLYLQMNSLRTED
TALYYCARYYRYDAWFAYWGQGTLVTVSS
(15D3hmzv16-1 hG1),

where H1CDRs1-3 are shown in bold.

In another embodiment, the first heavy chain variable region sequence comprises:

(SEQ ID NO: 9)
EVQLVESGGVVVQPGGSLRLSCAASGFTFSRYTMSWVRQAPGKGL
EWVATISSGGGNTYYPDSVKGRFTVSRDNSKNSLYLQMNSLRTED
TALYYCARYGAGDAWFAYWGQGTLVTVSS
(15D3 hmz);
or
(SEQ ID NO: 10)
EVQLVESGGVVVQPGGSLRLSCAASGFTFSRYTMSWVRQAPGKGL
EWVATISSGGGNTYYPDSVKGRFTVSRDNSKNSLYLQMNSLRTED
TALYYCARYYRYDAWFAYWGQGTLVTVSS
(15D3hmzv16-1 hG1).

In all embodiments, in the subject multi-specific IgG1 antibody molecules the TAA may, for example, be CD47, PD-L1, or EGFR, especially CD47.

When the TAA is CD47, the antigen-binding site of the second heavy chain variable region may comprise CDRs1-3 (H2CDRs1-3) of heavy chain variable region sequence:

(SEQ ID NO: 11)
QVQLVQSGAEVKKPGASVKVSCKASGYTFTNYNMHWVRQAPGQR
LEWMGTIYPGNDDTSYNQKFKDRVTITADTSASTAYMELSSLRS
EDTAVYYCARGGYRAMDYWGQGTLVTVSS,

and the second heavy chain variable region may comprise the sequence:

(SEQ ID NO: 11)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSS.

In all aspects and embodiments, the multi-specific IgG1 antibody molecules herein may exhibit at least one of decreased mispairing, decreased head-to-tail formation, decreased half-antibody production and increased overall yield of production as compared to an IgG1 antibody without Fc mutations or having only amino acid substitutions T366S, L368A, and Y407V in the first Fc region and only amino acid substitutions T366W in the second Fc region, wherein numbering is according to the EU numbering scheme.

In another aspect the invention concerns a bispecific humanized IgG1 antibody molecule that binds multidrug resistance ABCB1 and a TAA, the antibody molecule comprising two identical light chain variable regions, a first heavy chain variable region, and a second heavy chain variable region,

wherein the light chain variable regions each comprise an antigen-binding site for ABCB1, the first heavy chain variable region comprises an antigen-binding site for ABCB1, the second heavy chain variable region comprises an antigen-binding site for the TAA, and the second VH chain binds the TAA when paired with one of the VL chains, and

wherein the antigen-binding site of the two identical light chain variable regions comprises light chain CDRs 1-3 (LCDRs 1-3) of a light chain variable region sequence selected from the group consisting of:

(SEQ ID NO: 1)
DIVMTQTPLSSPVTLGQPASISCRSSQSIVHSTGNTYLEWYQQRPGQPPR
LLIYKISNRFSGVPDRFSGSGAGTDFTLKISRVEAEDVGVYYCFQASHFP
RTFGGGTKLEIKR
(MRK16 hmz);
(SEQ ID NO: 2)
DIVMTQSPLSLPVTPGEPASISCRSSQSLVHSNGNTYLEWYLQKPGQSPQ
LLIYKVSNRFSGVPDRFSGSGSGTDFTLKISRVEAEDVGVYYCFQGSHFP
RTFGQGTKLEIK
(B1VL6/CDR3v2a hmz);
(SEQ ID NO: 3)
DIVMTQTPLSSPVTLGQPASISCRSSQSIVHSTGNTYLEWYQQKPGQPPR
LLIYKISNRFSGVPDRFSGSGSGTDFTLKISRVEAEDVGVYYCFQASHFP
RTFGQGTKLEIK
(MRK16v4a1 hmz);
(SEQ ID NO: 4)
DIVMTQTPLSSPVTLGQPASISCRSSQSIVHSTGNTYLEWYQQKPGQPPR
LLIYKVSNRFSGVPDRFSGSGSGTDFTLKISRVEAEDVGVYYCFQASHFP
RTFGQGTKLEIK
(MRK16v4a12 hmz);
(SEQ ID NO: 6)
DIVMTQSPLSLPVTPGEPASISCRSSQTIVHSNGNTYLEWYLQKPGQSPQ
LLIYKVSKRFSGVPDRFSGSGSGTDFTLKISRVEAEDVGVYYCFQASHFP
RTFGQGTKLEIK
(B1.89v1.huL1 (KV2) hmz);
(SEQ ID NO: 7)
DVVLTQSPLSLPVTPGEPASISCRSSQNIVHSTGNTYLDWYLQKPGQSPQ
LLIYKVSNRFSGVPDRFSGSGSGTDFTLKISRVEAEDVGVYYCFQGSHIP
RTFGQGTKLEIKR
(B2.28.huL2);
and
(SEQ ID NO: 8)
DIVMTQSPLSLPVSLGDPASISCRSSQSLVHSNGNTYLEYYLQKPGQSPK
LLIYKVSNRFSGVPDRFSGSGSGTDFTLKISRVEAEDLGVYYCFQGSHFP
RTFGGGTKLEIK

(B1VL6/CDR3v2 hmz), wherein LCDRs 1-3 are shown in bold.

In one embodiment, the two identical light chain variable region sequences comprise a sequence selected from the group consisting of:

(SEQ ID NO: 1)
DIVMTQTPLSSPVTLGQPASISCRSSQSIVHSTGNTYLEWYQQRPGQPPR
LLIYKISNRFSGVPDRFSGSGAGTDFTLKISRVEAEDVGVYYCFQASHFP
RTFGGGTKLEIKR
(MRK16 hmz);
(SEQ ID NO: 2)
DIVMTQSPLSLPVTPGEPASISCRSSQSLVHSNGNTYLEWYLQKPGQSPQ
LLIYKVSNRFSGVPDRFSGSGSGTDFTLKISRVEAEDVGVYYCFQGSHFP
RTFGQGTKLEIK
(B1VL6/CDR3v2a hmz);
(SEQ ID NO: 3)
DIVMTQTPLSSPVTLGQPASISCRSSQSIVHSTGNTYLEWYQQKPGQPPR
LLIYKISNRFSGVPDRFSGSGSGTDFTLKISRVEAEDVGVYYCFQASHFP
RTFGQGTKLEIK
(MRK16v4a1 hmz);
(SEQ ID NO: 4)
DIVMTQTPLSSPVTLGQPASISCRSSQSIVHSTGNTYLEWYQQKPGQPPR
LLIYKVSNRFSGVPDRFSGSGSGTDFTLKISRVEAEDVGVYYCFQASHFP
RTFGQGTKLEIK
(MRK16v4a12 hmz);
(SEQ ID NO: 6)
DIVMTQSPLSLPVTPGEPASISCRSSQTIVHSNGNTYLEWYLQKPGQSPQ
LLIYKVSKRFSGVPDRFSGSGSGTDFTLKISRVEAEDVGVYYCFQASHFP
RTFGQGTKLEIK
(B1.89v1.huL1 (KV2) hmz);
(SEQ ID NO: 7)
DVVLTQSPLSLPVTPGEPASISCRSSQNIVHSTGNTYLDWYLQKPGQSPQ
LLIYKVSNRFSGVPDRFSGSGSGTDFTLKISRVEAEDVGVYYCFQGSHIP
RTFGQGTKLEIKR
(B2.28.huL2);
and
(SEQ ID NO: 8)
DIVMTQSPLSLPVSLGDPASISCRSSQSLVHSNGNTYLEYYLQKPGQSPK
LLIYKVSNRFSGVPDRFSGSGSGTDFTLKISRVEAEDLGVYYCFQGSHFP
RTFGGGTKLEIK
(B1VL6/CDR3v2 hmz).

In a particular embodiment, in the bispecific humanized IgG1 antibody molecule the antigen-binding site of the two identical light chain variable regions comprises light chain CDRs 1-3 (LCDRs 1-3) of a light chain variable region sequence selected from the group consisting of:

(SEQ ID NO: 6)
DIVMTQSPLSLPVTPGEPASISCRSSQTIVHSNGNTYLEWYLQKPGQSPQ
LLIYKVSKRFSGVPDRFSGSGSGTDFTLKISRVEAEDVGVYYCFQASHFP
RTFGQGTKLEIK
(B1.89v1.huL1 (KV2) hmz);
(SEQ ID NO: 7)
DVVLTQSPLSLPVTPGEPASISCRSSQNIVHSTGNTYLDWYLQKPGQSPQ
LLIYKVSNRFSGVPDRFSGSGSGTDFTLKISRVEAEDVGVYYCFQGSHIP
RTFGQGTKLEIKR
(B2.28.huL2);
and
(SEQ ID NO: 8)
DIVMTQSPLSLPVSLGDPASISCRSSQSLVHSNGNTYLEYYLQKPGQSPK
LLIYKVSNRFSGVPDRFSGSGSGTDFTLKISRVEAEDLGVYYCFQGSHFP
RTFGGGTKLEIK

(B1VL6/CDR3v2 hmz), wherein LCDRs 1-3 are shown in bold.

In another particular embodiment, in the bispecific humanized IgG1 antibody molecule the two identical light chain variable region sequences comprise the sequence of:

(SEQ ID NO: 6)
DIVMTQSPLSLPVTPGEPASISCRSSQTIVHSNGNTYLEWYLQKPGQSPQ
LLIYKVSKRFSGVPDRFSGSGSGTDFTLKISRVEAEDVGVYYCFQASHFP
RTFGQGTKLEIK
(B1.89v1.huL1 (KV2) hmz);
(SEQ ID NO: 7)
DVVLTQSPLSLPVTPGEPASISCRSSQNIVHSTGNTYLDWYLQKPGQSPQ
LLIYKVSNRFSGVPDRFSGSGSGTDFTLKISRVEAEDVGVYYCFQGSHIP
RTFGQGTKLEIKR
(B2.28.huL2);
or
(SEQ ID NO: 8)
DIVMTQSPLSLPVSLGDPASISCRSSQSLVHSNGNTYLEYYLQKPGQSPK
LLIYKVSNRFSGVPDRFSGSGSGTDFTLKISRVEAEDLGVYYCFQGSHFP
RTFGGGTKLEIK
(B1VL6/CDR3v2 hmz).

In some embodiments, in the bispecific humanized IgG1 antibody molecule the antigen-binding site of the first heavy chain variable region comprises heavy chain CDRs 1-3 (H1CDRs 1-3) of a heavy chain variable region sequence selected from the group consisting of:

(SEQ ID NO: 9)
EVQLVESGGVVVQPGGSLRLSCAASGFTFSRYTMSWVRQAPGKGLEWVAT
ISSGGGNTYYPDSVKGRFTVSRDNSKNSLYLQMNSLRTEDTALYYCARYG
AGDAWFAYWGQGTLVTVSS
(15D3 hmz);
and
(SEQ ID NO: 10)
EVQLVESGGVVVQPGGSLRLSCAASGFTFSRYTMSWVRQAPGKGLEWVAT
ISSGGGNTYYPDSVKGRFTVSRDNSKNSLYLQMNSLRTEDTALYYCARYY
RYDAWFAYWGQGTLVTVSS
(15D3hmzv16-1 hG1),

where H1CDRs1-3 are shown in bold.

In other embodiments, in the bispecific humanized IgG1 antibody molecule the first heavy chain variable region sequence comprises:

(SEQ ID NO: 9)
EVQLVESGGVVVQPGGSLRLSCAASGFTFSRYTMSWVRQAPGKGLEWVAT
ISSGGGNTYYPDSVKGRFTVSRDNSKNSLYLQMNSLRTEDTALYYCARYG
AGDAWFAYWGQGTLVTVSS
(15D3 hmz);
or
(SEQ ID NO: 10)
EVQLVESGGVVVQPGGSLRLSCAASGFTFSRYTMSWVRQAPGKGLEWVAT
ISSGGGNTYYPDSVKGRFTVSRDNSKNSLYLQMNSLRTEDTALYYCARYY
RYDAWFAYWGQGTLVTVSS
(15D3hmzv16-1 hG1).

In all embodiments, in the bispecific humanized IgG1 antibody molecule the TAA may, for example, be CD47, PD-L1, or EGFR, especially CD47.

When the TAA is CD47, in the subject bispecific humanized IgG1 antibody molecule the antigen-binding site of the second heavy chain variable region may comprise CDRs1-3 (H2CDRs1-3) of heavy chain variable region sequence:

(SEQ ID NO: 11)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSS.

and the second heavy chain variable region may comprise the sequence:

(SEQ ID NO: 11)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSS.

In all embodiments concerning the bispecific humanized IgG1 antibody molecules disclosed herein, the first and the second heavy chains may each comprise an Fc region, and one of the Fc regions comprises hole amino acid substitutions Y349C, T366S, L368A, and Y407V, optionally in combination with amino acid substitution F405V or Q347R, wherein the numbering is according to the EU numbering scheme.

In certain embodiments, the other Fc region comprises knob amino acid substitutions S354C and T366W, wherein numbering is according to the EU numbering scheme.

In another embodiment, one of the Fc regions further comprises one or both of amino acid substitutions D399K and E356K, while the other Fc region may further comprise one or both of amino acid substitutions K409D and K392D, wherein numbering is according to the EU numbering scheme.

In certain bispecific humanized IgG1 antibody molecules herein, one of the Fc regions comprises a set of amino acid substitutions and amino acid residues selected from the group consisting of:
(a) Y349C, T366S, L368A, Y407V, E356, E357, F405V;
(b) Y349C, T366S, L368A, Y407V, E356, E357, F405V, K409D;
(c) Y349C, T366S, L368A, Y407V, E356, E357, K409D;
(d) Y349C, T366S, L368A, Y407V, E356, E357, D399K;
(e) Y349C, T366S, L368A, Y407V, E356, E357, F405V, D399K; and
(f) Y349C, T366S, L368A, Y407V, E356, E357, Q347R, wherein numbering is according to the EU numbering scheme.

In some bispecific humanized IgG1 antibody molecules, the other Fc region comprises a set of amino acid substitutions and amino acid residues selected from the group consisting of:
(a) S354C, T366W, E356, E357;
(b) S354C, T366W, E356, E357, D399K;
(c) S354C, T366W, E356, E357, K409D;
(d) S354C, T366W, E356, E357, K360E,

wherein numbering is according to the EU numbering scheme.

In a further group of bispecific humanized IgG1 antibody molecules, one of the Fc regions comprises the set of amino acid substitutions and amino acid residues Y349C, T366S, L368A, Y407V, E356, E357, F405V, and the other Fc region comprises the set of amino acid substitutions and amino acid residues S354C, T366W, E356, E357, wherein numbering is according to the EU numbering scheme.

In another group of bispecific humanized IgG1 antibody molecule herein, one of the Fc regions comprises the set of amino acid substitutions and amino acid residues Y349C, T366S, L368A, Y407V, E356, E357, K409D, and the other Fc region comprises the set of amino acid substitutions and amino acid residues S354C, T366W, E356, E357, D399K, wherein numbering is according to the EU numbering scheme.

In further bispecific humanized IgG1 antibody molecules herein, one of the Fc regions comprises the set of amino acid substitutions and amino acid residues Y349C, T366S, L368A, Y407V, E356, E357, F405V, D399K, and the other Fc region comprises the set of amino acid substitutions and amino acid residues S354C, T366W, E356, E357, K409D, wherein numbering is according to the EU numbering scheme.

In still further bispecific humanized IgG1 antibody molecules herein, one of the Fc regions comprises the set of amino acid substitutions and amino acid residues Y349C, T366S, L368A, Y407V, E356, E357, Q347R, and the other Fc region comprises the set of amino acid substitutions and amino acid residues S354C, T366W, E356, E357, K360E, wherein numbering is according to the EU numbering scheme.

Included herein is a bispecific humanized IgG1 antibody molecule, wherein the first 15 heavy chain comprises an Fc region of the following sequence:

(SEQ ID NO: 12)
HC1 (hG1-HIR2):
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVCTLPPSREEMTKNQVSLSC
AVKGFYPSDIAVEWESNGQPENNYKTTPPVLDSDGSFVLVSKLTVDKSRW
QQGNVFSCSVMHEALHNHYTQKSLSLSPGK

and the second heavy chain comprises an Fc region of the following sequence:

(SEQ ID NO: 22)
HC1 (hG1-KbR1):
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPCREEMTKNQVSLWC
LVKGFYPSDIAVEWESNGQPENNYKTTPPVLDSDGSFFLYSKLTVDKSRW
QQGNVFSCSVMHEALHNHYTQKSLSLSPGK

wherein the amino acid residues facilitating pairing are shown in bold.

Further included herein is a bispecific humanized IgG1 antibody molecule, wherein the first heavy chain comprises an Fc region of the following sequence:

(SEQ ID NO: 16)
HC1 (hG1-HIR4):
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVCTLPPSREEMTKNQVSLSC
AVKGFYPSDIAVEWESNGQPENNYKTTPPVLDSDGSFFLVSDLTVDKSRW
QQGNVFSCSVMHEALHNHYTQKSLSLSPGK

and the second heavy chain comprises an Fc region of the following sequence:

(SEQ ID NO: 23)
HC2 (hG1- KbR3):
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPCREEMTKNQVSLWC
LVKGFYPSDIAVEWESNGQPENNYKTTPPVLKSDGSFFLYSKLTVDKSRW
QQGNVFSCSVMHEALHNHYTQKSLSLSPGK

wherein the amino acid residues facilitating pairing are shown in bold.

Also included herein is a bispecific humanized IgG1 antibody molecule, wherein the first heavy chain comprises an Fc region of the following sequence:

(SEQ ID NO: 18)
HC1 (hG1-HIR6):
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVCTLPPSREEMTKNQVSLSC
AVKGFYPSDIAVEWESNGQPENNYKTTPPVLKSDGSFVLVSKLTVDKSRW
QQGNVFSCSVMHEALHNHYTQKSLSLSPGK

and the second heavy chain comprises an Fc region of the following sequence:

(SEQ ID NO: 24)
HC2 (hG1-KbR5):
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPCREEMTKNQVSLWC
LVKGFYPSDIAVEWESNGQPENNYKTTPPVLDSDGSFFLYSDLTVDKSRW
QQGNVFSCSVMHEALHNHYTQKSLSLSPGK

wherein the amino acid residues facilitating pairing are shown in bold.

In another bispecific humanized IgG1 antibody molecule, the first heavy chain comprises an Fc region of the following sequence:

HC1 (hG1-HIR7):
(SEQ ID NO: 19)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVCTLPPSREEMTKNQVSLSC
AVKGFYPSDIAVEWESNGQPENNYKTTPPVLDSDGSFFLVSKLTVDKSRW
QQGNVFSCSVMHEALHNHYTQKSLSLSPGK

and the second heavy chain comprises an Fc region of the following sequence:

HC2 (hG1-KbR7):
(SEQ ID NO: 25)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPCREEMTENQVSLWC
LVKGFYPSDIAVEWESNGQPENNYKTTPPVLDSDGSFFLYSKLTVDKSRW
QQGNVFSCSVMHEALHNHYTQKSLSLSPGK

wherein the amino acid residues facilitating pairing are shown in bold.

In one aspect, the bispecific humanized IgG1 antibody molecule exhibits at least one of decreased mispairing, decreased head-to-tail formation, decreased half-antibody production and increased overall yield of production as compared to an IgG1 antibody without Fc mutations or having only amino acid substitutions T366S, L368A, and Y407V in the first Fc region and only amino acid substitutions T366W in the second Fc region, wherein numbering is according to the EU numbering scheme.

In another aspect, the invention concerns a humanized antibody light chain binding to ABCB1 comprising CDRs 1-3 (LCDRs 1-3) of a light chain variable region sequence selected from the group consisting of:

(SEQ ID NO: 1)
DIVMTQTPLSSPVTLGQPASISCRSSQSIVHSTGNTYLEWYQQRPGQPPR
LLIYKISNRFSGVPDRFSGSGAGTDFTLKISRVEAEDVGVYYCFQASHFP
RTFGGGTKLEIKR (MRK16 hmz);
(SEQ ID NO: 2)
DIVMTQSPLSLPVTPGEPASISCRSSQSLVHSNGNTYLEWYLQKPGQSPQ
LLIYKVSNRFSGVPDRFSGSGSGTDFTLKISRVEAEDVGVYYCFQGSHFP
RTFGQGTKLEIK (B1VL6/CDR3v2a hmz);
(SEQ ID NO: 3)
DIVMTQTPLSSPVTLGQPASISCRSSQSIVHSTGNTYLEWYQQKPGQPPR
LLIYKISNRFSGVPDRFSGSGSGTDFTLKISRVEAEDVGVYYCFQASHFP
RTFGQGTKLEIK (MRK16v4a1 hmz);
(SEQ ID NO: 4)
DIVMTQTPLSSPVTLGQPASISCRSSQSIVHSTGNTYLEWYQQKPGQPPR
LLIYKVSNRFSGVPDRFSGSGSGTDFTLKISRVEAEDVGVYYCFQASHFP
RTFGQGTKLEIK (MRK16v4a12 hmz);
(SEQ ID NO: 6)
DIVMTQSPLSLPVTPGEPASISCRSSQTIVHSNGNTYLEWYLQKPGQSPQ
LLIYKVSKRFSGVPDRFSGSGSGTDFTLKISRVEAEDVGVYYCFQASHFP
RTFGQGTKLEIK (B1.89v1.huL1 (KV2) hmz);
(SEQ ID NO: 7)
DVVLTQSPLSLPVTPGEPASISCRSSQNIVHSTGNTYLDWYLQKPGQSPQ
LLIYKVSNRFSGVPDRFSGSGSGTDFTLKISRVEAEDVGVYYCFQGSHIP
RTFGQGTKLEIKR (B2.28.huL2);
and
(SEQ ID NO: 8)
DIVMTQSPLSLPVSLGDPASISCRSSQSLVHSNGNTYLEYYLQKPGQSPK
LLIYKVSNRFSGVPDRFSGSGSGTDFTLKISRVEAEDLGVYYCFQGSHFP
RTFGGGTKLEIK (B1VL6/CDR3v2 hmz),

wherein LCDRs 1-3 are shown in bold.

In a further embodiment, the humanized antibody light chain comprising a sequence selected from the group consisting of:

(SEQ ID NO: 1)
DIVMTQTPLSSPVTLGQPASISCRSSQSIVHSTGNTYLEWYQQRPGQPPR
LLIYKISNRFSGVPDRFSGSGAGTDFTLKISRVEAEDVGVYYCFQASHFP
RTFGGGTKLEIKR (MRK16 hmz);
(SEQ ID NO: 2)
DIVMTQSPLSLPVTPGEPASISCRSSQSLVHSNGNTYLEWYLQKPGQSPQ
LLIYKVSNRFSGVPDRFSGSGSGTDFTLKISRVEAEDVGVYYCFQGSHFP
RTFGQGTKLEIK (B1VL6/CDR3v2a hmz);
(SEQ ID NO: 3)
DIVMTQTPLSSPVTLGQPASISCRSSQSIVHSTGNTYLEWYQQKPGQPPR
LLIYKISNRFSGVPDRFSGSGSGTDFTLKISRVEAEDVGVYYCFQASHFP
RTFGQGTKLEIK (MRK16v4a1 hmz);
(SEQ ID NO: 4)
DIVMTQTPLSSPVTLGQPASISCRSSQSIVHSTGNTYLEWYQQKPGQPPR
LLIYKVSNRFSGVPDRFSGSGSGTDFTLKISRVEAEDVGVYYCFQASHFP
RTFGQGTKLEIK (MRK16v4a12 hmz);
(SEQ ID NO: 6)
DIVMTQSPLSLPVTPGEPASISCRSSQTIVHSNGNTYLEWYLQKPGQSPQ
LLIYKVSKRFSGVPDRFSGSGSGTDFTLKISRVEAEDVGVYYCFQASHFP
RTFGQGTKLEIK (B1.89v1.huL1 (KV2) hmz);
(SEQ ID NO: 7)
DVVLTQSPLSLPVTPGEPASISCRSSQNIVHSTGNTYLDWYLQKPGQSPQ
LLIYKVSNRFSGVPDRFSGSGSGTDFTLKISRVEAEDVGVYYCFQGSHIP
RTFGQGTKLEIKR (B2.28.huL2);
and
(SEQ ID NO: 8)
DIVMTQSPLSLPVSLGDPASISCRSSQSLVHSNGNTYLEYYLQKPGQSPK
LLIYKVSNRFSGVPDRFSGSGSGTDFTLKISRVEAEDLGVYYCFQGSHFP
RTFGGGTKLEIK (B1VL6/CDR3v2 hmz).

Humanized anti-ABCB1 antibodies comprising such humanized light chain are also included. Such antibodies include multi-specific and bispecific antibodies, which may comprise an antigen-binding site for a TAA, which may, for example, be CD47, PD-L1 or EGFR.

In certain embodiments, the antibodies disclosed herein, e.g., the bispecific antibodies disclosed herein induce ADCC. In certain embodiments, the antibodies, including bispecific antibodies, disclosed herein induce ADCC in a dose-dependent manner.

In certain embodiments, the antibodies disclosed herein, e.g., the bispecific antibodies disclosed herein able to recruit immune effector cells to kill target cells. In certain embodiments, the antibodies, including bispecific antibodies, disclosed herein recruit immune effector cells to kill target cells in a dose-dependent manner.

In a further aspect, provided herein is a composition comprising a multi-specific IgG1 antibody molecule, a bispecific humanized IgG1 antibody molecule, or a humanized anti-ABCB1 antibody molecule hereinabove described, in combination with a carrier. The composition may be a pharmaceutical composition.

Further included are methods of treatment with and uses of the multi-specific IgG1 antibody molecules, bispecific humanized IgG1 antibody molecules, and humanized anti-ABCB1 antibody molecules herein. The use or treatment may be any condition where control, especially reduction, of ABCB1 influx is desirable such as, for example, treatment of a cancer patient, such as patient with a drug resistant or chemotherapy resistant cancer.

The treatment methods may include administration of at least one additional active agent, wherein the at least one additional active agent may, for example, comprise a chemotherapeutic agent, an inhibitor of a multidrug resistance transporter, an immunotherapy agent, or a combination thereof. Examples of a chemotherapeutic agent are, without limitation, taxol, a vinca alkaloid, or an anthracycline. Drug resistance may develop following prior treatment with a chemotherapeutic agent or an immunotherapy agent, and in certain embodiments, the cancer may be resistant to an inhibitor of a multidrug resistance transporter.

The treatment methods and uses may include administration of the antibodies herein as a single agent or in combination with at least one further active agent, such as a chemotherapy agent, inhibitor of a multidrug resistance transporter or immunotherapy agent.

In some embodiments, the treatment increases the effectiveness of the at least one additional active agent as compared to treatment with the at least one additional active agent alone. The increased effectiveness may, for example, comprises an at least 5% increase in cancer cell killing.

Further provided herein are one or more nucleic acids comprising one or more sequences encoding the subject antibody molecules, optionally operably linked to a promoter, expression vectors comprising such nucleic acids, and mammalian cells genetically modified with such nucleic acids.

In a further aspect, a kit comprising a subject antibody or nucleic acid is provided, which may optionally comprise at least one additional active agent and/or instructions of use.

### BRIEF DESCRIPTION OF THE FIGURES

FIG. 1 schematically depicts a bispecific monoclonal antibody structure as described herein. Structure A: illustrates a bispecific antibody with mutations (HC DD/HC KK) in the heavy chain Fc regions leading to electrostatic steering effects and a common light chain. Structure B: illustrates a bispecific antibody with knob-into-hole mutations in the heavy chain Fc regions (HC HIR/HC KbR) and a common light chain.

FIGS. 2A and 2B illustrate various alternative IgG-based bispecific antibody structures within the scope herein. Although K-i-H mutations are not shown, they might be present in any of the structures illustrated. (GGGGS)ₙ (SEQ ID NO:40) and GSTGGGS(GGGGS)ₙ (SEQ ID NO:41) are flexible peptide linkers commonly used in protein engineering.

FIG. 3 shows the results of testing the purity of the ABCB1XCD47 KBisP1.1 (15D3 HC KK/KT14 HC DD/MRK16 LC) bispecific antibody by SEC-HPLC and CIEX-HPLC after Protein A purification and Capto-MMC polishing.

FIG. 4 shows the results of testing the purity of various HIRX 15D3/KbRY KT14/MRK16 LC ABCB1xCD47 bispecific antibodies by SEC-HPLC and CIEX-HPLC after Protein A purification. “X” stands for various HIR mutations and “Y” stands for various KbR mutations, as shown.

FIG. 5 shows the results of testing the purity of various HIRX 15D3/KbRY KT14/B1VL6/CDR3v2 hmz LC ABCB1XCD47 bispecific antibodies by SEC-HPLC and CIEX-HPLC after Protein A purification. “X” stands for various HIR mutations and “Y” stands for various KbR mutations, as shown.

FIG. 6 shows the results of testing the purity of various HIRX 15D3/KbRY KT14/KNJY_B2.28.huL2 LC bispecific antibodies by SEC-HPLC and CIEX-HPLC after Protein A purification. “X” stands for various HIR mutations and “Y” stands for various KbR mutations, as shown.

FIG. 7 presents the results of formulation studies with the ABCB1XCD47 bispecific antibody KBisP1.1 (15D3 HC KK/KT14 HC DD/MRK16 LC). T=Trehalose, NaOAc=sodium acetate, PBS=phosphate-buffered saline.

FIG. 8 presents the results of (A) & (B): long-term stability studies with the ABCB1XCD47 bispecific antibody KBisP1.1 (15D3 HC KK/KT14 HC DD/MRK16 LC) at 40° C. at 1 mg/ml in 20 mM Histidine, pH6.0+100 mM Trehalose (T), and (C) & (D): thermostability studies with the ABCB1XCD47 bispecific antibody KBisP1.1 (15D3 HC KK/KT14 HC DD/MRK16 LC) at 1 mg/ml Histidine, pH6.0+100 mM Trehalose (T).

FIG. 9 presents the results of thermostability studies with the bispecific antibody ABCB1XCD47 KBisP1.1 (15D3 HC KK/KT14 HC DD/MRK16 LC) at 1 and 10 mg/ml in 20 mM Histidine, pH 6.0+100 mM Trehalose (T).

FIG. 10 presents the results of freeze/thaw stability studies with the bispecific antibody ABCB1XCD47 (15D3 HC KK/KT14 HC DD/MRK16 LC) at 1 and 10 mg/ml in 20 mM Histidine, pH 6.0+100 mM Trehalose (T).

FIG. 11 presents the results of thermostability studies with ABCB1XCD47 bispecific antibody 15D3 HIR2/KT14 KbR1 and 15D3 HIR4/KT14 KbR3 with two different light chains: B1VL6/CDR3v2a and B1.89v1.huL1(KV2) at 1 mg/ml in 20 mM Histidine, pH 6.0+100 mM Trehalose.

FIG. 12 presents the results of long-term stability studies of bispecific ABCB1XCD47 antibodies 15D3 HIR2/KT14 KbR1 and 15D3 HIR4/KT14 KbR3 paired with two different light chains (LCs): B1 VL6/CDR3v2a and B1.89v1.huL1(KV2) at 1 mg/ml in 20 mM Histidine, pH 6.0+10 mM Trehalose.

FIG. 13 presents the results of freeze/thaw studies at 1 mg/ml of bispecific ABCB1XCD47 antibodies 15D3 HIR2/KT14 KbR1 and 15D3 HIR4/KT14 KbR3 paired with two different light chains (LCs) B1VL6/CDR3v2a and B1/89v1.huL1(KV2) at 1 mg/ml in 20 mM Histidine, pH 6.0+100 mM Trehalose.

FIG. 14 shows the binding of different formats of ABCB1XCD47 bispecific antibodies to the extracellular domain (ECD) of huCD47 tested by ELISA. For further details of the structures see also FIGS. 2A and 2B. The binding of the different formats was compared to binding of 5F9 and the 15D3 hmz DD/KT14 KK/MRK16 hmz bispecific antibodiesin the IgG1 format.

FIG. 15A shows the binding of ABCB1XCD47 bispecific antibodies with K-i-H and modified K-i-H Fc region, tested by A: ELISA for binding to the extracellular domain (CD) of huCD47 and B: by FACS for binding to the HEK 293T naïve cell line expressing a high level of hCD47 and moderately low level of ABCB1.

FIG. 15B shows the binding of ABCB1XCD47 bispecific antibodies to ABCB1 over-expressing HEK 293T cells (C); and HEK 293T CD47 KO cells (D).

FIG. 16 shows the binding of ABCB1XCD47 bispecific antibodies 15D3hmzG1 HIR2/KT14hG1 KbR1/MRK16v4b hmz polished, 15D3hmzG1 HIR2/KT14 hG1 KbR1/B1VL6/CDR3v2a hmz LC polished, and 15D3hmzG1 HIR2/KT14 hG1 KbR1/B1.89v1 huL1(KV2) hmz LC polished to wild-type DX5 WT cell lines.

FIG. 17 shows the binding of ABCB1XCD47 bispecific antibodies 15D3hmzG1 HIR2/KT14hG1 KbR1/MRK16v4b hmz polished, 15D3hmzG1 HIR2/KT14 hG1 KbR1/B1VL6/CDR3v2a hmz LC polished, and 15D3hmzG1 HIR2/KT14 hG1 KbR1/B1.89v1 huL1(KV2) hmz LC polished to CD47 KO DX5 cell lines.

FIG. 18 shows the binding of ABCB1XCD47 bispecific antibodies 15D3hmzG1 HIR2/KT14hG1 KbR1/MRK16v4b hmz polished, 15D3hmzG1 HIR2/KT14 hG1 KbR1/B1VL6/CDR3v2a hmz LC polished, and 15D3hmzG1 HIR2/KT14 hG1 KbR1/B1.89v1 huL1(KV2) hmz LC polished to ABCB1 KO DX5 cell lines.

FIGS. 19A-19C show that humanized ABCB1XCD47 bispecific antibodies KB1-1418, KB1-1419 and KB1-1420 specifically bind to ABCB1 and CD47 antigens expressed on cancer cell surface, using wild-type Adriamycin-resistant DX5/ADR CD47 WT, DX5 CD47 KO and DX5 ABCB1 KO (B1 KO) cell lines.

FIGS. 20A-20B show the binding profile of humanized ABCB1XCD47 bispecific antibodies KB1-1418, KB1-1419, and KB1-1420, and non-humanized ABCB1XCD47 bispecific antibody KB1-1401 in a receptor occupancy assay, using the multidrug resistant ovarian carcinoma tumor cell line A2780ADR.

FIG. 21 shows a schematic depicting receptor occupancy assay.

FIG. 22 presents the results of in vitro potency assays in ABCB1 expressing N6ADR (leukemia) cell lines performed with the ABCB1XCD47 bispecific antibodies 15D3hmzG1 HIR2/KT14 hG1 KbR1/B1VL6/CDR3v2a hmz LC and 15D3hmzG1 HIR2/KT14hG1 KbR1/B1.89.v1 huL1(KV2) hmz LC relative to bispecific ABCB1XCD47 antibody KBisP1.1, and anti-ABCB1 and anti-CD47 mono-arm antibodies, having the same light chain as the corresponding bispecific antibodies.

FIGS. 23A-23B present results showing that humanized bispecific ABCB1 X CD47 antibodies KB1-1418, KB1-1419 and KB1-1420 show significant in vitro potency in a N6ADR drug resistant cell-based assay. FIG. 24 shows that the tested bispecific ABCB1XCD47 antibodies inhibit efflux in Paclitaxel treated 293T hB1 cells overexpressing ABCB1 antigen.

FIG. 25 shows that humanized bispecific ABCB1XCD47 bispecific antibody KB1-1420 demonstrates clear efflux inhibition in paclitaxel treated HEK293T ABCB1 overexpressing stable cell line.

FIG. 26. Flow cytometry gating for assessing antibody binding to RBCs.

FIG. 27 presents results of assays testing the in vivo efficacy and dose-response of bispecific ABCB1XCD47 antibodies in the SA-MES-DX5 model. BisP1.1 and 15D3 HIR2/KT14 KbR1/B1.89v1.huL1 (KV2) hmz LC and 15D316-1 DD/KT14 KK/MRK16v4a1 hmz LC bispecific antibodies significantly inhibited tumor growth of SA-MES-DX5 cells as single agents and in combination with paclitaxel.

FIG. 28 presents results of assays testing the in vivo efficacy and dose-response of bispecific ABCB1XCD47 antibody 15D3 HIR2/KT14 KbR1/B1.89v1.huL1 (KV2) hmz LC in the A2780ADR tumor model.

FIG. 29 presents results of Antibody Dependent Cell Cytotoxicity (ADCC) assessment of bispecific ABCB1XCD47 antibody 15D3 HIR2/KT14 KbR1/B1.89v1.huL1 (KV2) hmz LC against the human MES-SA/DX5 multidrug resistant cell model target.

FIG. 30 presents data showing that humanized bispecific ABCB1XCD47 antibody KB1-1420 significantly affects tumor growth rates, slowing down tumor progression in the multidrug resistant ovarian adenocarcinoma A2780ADR tumor model in vivo.

FIG. 31 shows that KB1-1420 combined with paclitaxel shows strong therapeutic effect against human xenograft tumors in nude mice.

FIG. 32 show that KB1-1420 exhibits negligible binding to human red blood cells (RBCs) compared to an anti-CD47 monoclonal antibody.

FIG. 33 presents results of a potency assay performed in the multidrug resistant uterine sarcoma MES-SA/DX5/ADDR tumor model in vivo. The results demonstrate that KB1-1420, with or without paclitaxel, significantly enhances survival in a dose-dependent fashion.

FIGS. 34A-34B show that KB1-1420 is efficacious as a single agent and enhances paclitaxel-mediated cell killing in multidrug resistant ovarian adenocarcinoma cell line A2780ADR tumor model in vivo.

### DEFINITIONS

Throughout the disclosure, the terms “MDR1,” “ABCB1,” “Pgp,” P-glycoprotein-1, and “KPB1” are used herein interchangeably.

The term “tumor-associated antigen” or “TAA” is used herein in the broadest sense and includes tumor-specific antigens (TSA) that are exclusively expressed by tumor cells and antigens that are preferentially expressed by tumor cells but are also found in normal cells. Tumor associated antigens (TAAs) that can be targeted by the ABCB1 X TAA bispecific antibodies disclosed herein include, without limitation, TAAs that are overexpressed in drug-resistant tumors, especially multidrug-resistant (MDR) tumors, and/or are co-expressed with ABCB1 on tumor cells. Such TAAs include, without limitation, the tumor-associated antigens listed in Table 4, and specifically CD47, HER2, EGFR, PD-L1, AXL, B7-H4, LIV1, LY6E, LRRC15, Nectin4, TIM3, gpNMB, Fuc-GM1, cMET, ENPP3, CD19, CD20, CD27, CD30, CD33. D44, CD66e, CD70, CD73, CD79b, CD115, CD221, and CD228.

The terms “antibody” and “immunoglobulin” and their grammatical variants include antibodies or immunoglobulins of any isotype, fragments of antibodies which retain specific binding to antigen, including, but not limited to, Fab, Fv, scFv, and Fc fragments, chimeric antibodies, humanized antibodies, single-chain antibodies, including antibodies comprising only heavy chains (e.g. VHH camelid antibodies), bispecific antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the term are Fab′, Fv, F(ab′)₂, and or other antibody fragments that retain specific binding to antigen, and monoclonal antibodies.

An antibody may be monovalent or bivalent. The antibodies used herein may be used to assay expression of a target antigens(s) on a cell surface, e.g., in a cell sample or a tissue sample from a patient.

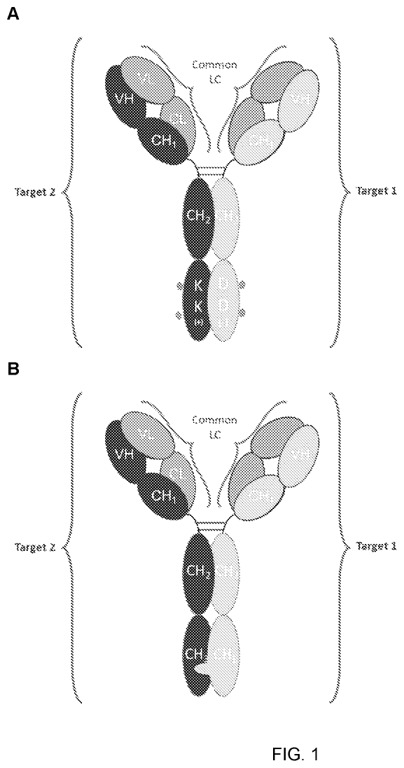

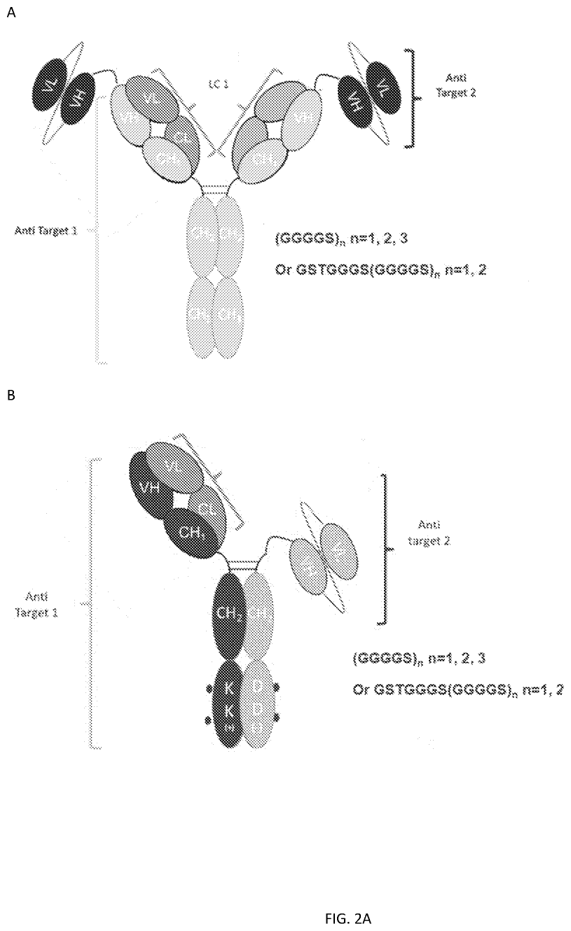

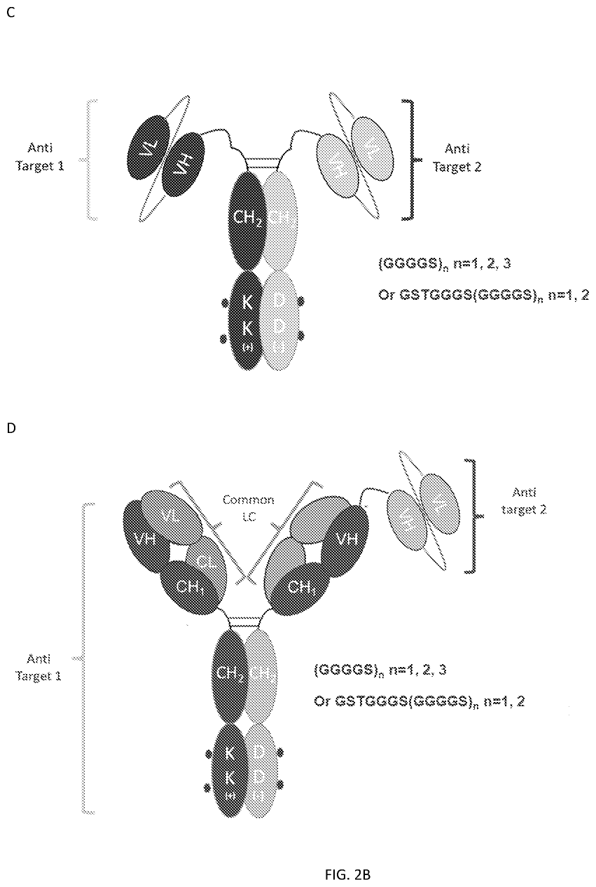

As used herein the term “antibody” encompasses, but is not limited to, a tetramer of two heavy and two light chains, wherein the heavy and light chains are interconnected by, for example, disulphide bonds. The heavy chain constant region is comprised of three domains, CH1, CH2 and CH3. The light chain constant region is comprised of one domain, CL. The variable regions of the heavy and light chains comprise binding regions that interact with antigen. The constant regions of the antibodies typically mediate the binding of the antibody to host tissues and factors, including various cells of the immune system and the first component of the complement system. The term “antibody” includes immunoglobulins of types IgA, IgG, IgE, IgD, IgM and subtypes thereof. In some embodiments, a subject antibody is an IgG isotype, i.e., IgG1, IgG, IgG3, IgG4, e.g., IgG1. Various antibody formats are illustrated in FIG. 1 and FIGS. 2A and 2B.

As used herein the term “immunoglobulin” refers to a protein including one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes; and numerous immunoglobulin variable region genes. Full-length immunoglobulin light chains (about 25 kD or 214 amino acids) are encoded by a variable region gene at the N-terminus (about 110 amino acids) and a kappa or lambda constant region at the C-terminus. Full-length immunoglobulin heavy chains (about 50 kD or 446 amino acids) are encoded by a variable region gene at the N-terminus (about 116 amino acids) and one of the other aforementioned constant region genes at the C-terminus, e.g. gamma (encoding about 330 amino acids). In some embodiments, a subject antibody comprises a whole immunoglobulin comprising full-length immunoglobulin heavy chain and a full-length immunoglobulin light chain.

The term “multispecific antibody” is used in the broadest sense and specifically covers an antibody that has polyepitopic specificity. Such multispecific antibodies include, but are not limited to, an antibody comprising a heavy chain variable domain (VH) and a light chain variable domain (VL), wherein the VH-VL unit has polyepitopic specificity, antibodies having two or more VL and VH domains with each VH-VL unit binding to a different epitope, antibodies having two or more single variable domains with each single variable domain binding to a different epitope, full length antibodies, antibody fragments such as Fab, Fv, dsFv, scFv, diabodies, bispecific diabodies and triabodies, antibody fragments that have been linked covalently or non-covalently. “Polyepitopic specificity” refers to the ability to specifically bind to two or more different epitopes on the same or different target(s). “Monospecific” refers to the ability to bind only one epitope. According to one embodiment the multispecific antibody is an IgG antibody that binds to each epitope with an affinity of 5 μM to 0.001 pM, 3 μM to 0.001 pM, 1 μM to 0.001 pM, 0.5 kM to 0.001 pM, or 0.1 kM to 0.001 pM. The term “multispecific” specifically includes “bispecific.” The term “antigen-binding fragment” refers to one or more fragments of a full-length antibody that are capable of specifically binding to an antigen. Examples of binding fragments include (i) a Fab fragment (a monovalent fragment including, e.g., consisting of, the VL, VH, CL and CH1 domains; (ii) a F(ab′)₂ fragment (a bivalent fragment comprising two Fab fragments linked by a disulfide bridge at the hinge region; (iii) a Fd fragment (including, e.g., consisting of, the VH and CH1 domains); (iv) a Fv fragment (including, e.g., consisting of, the VH and VL domains of a single arm of an antibody); (v) a dAb fragment (including, e.g., consisting of, the VH domain); (vi) an isolated CDR; (vii) a single chain Fv (scFv) (including, e.g., consisting of, the VH and VL domains of a single arm of an antibody joined by a synthetic linker using recombinant means such that the VH and VL domains pair to form a monovalent molecule); (viii) diabodies (including, e.g., consisting of, two scFvs in which the VH and VL domains are joined such that they do not pair to form a monovalent molecule; the VH of each one of the scFv pairs with the VL domain of the other scFv to form a bivalent molecule).

The term “chimeric” antibody refers to an antibody in which a portion of the heavy and/or light chain is derived from a particular source or species, while the remainder of the heavy and/or light chain is derived from a different source or species.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework (FR) which represents the most commonly occurring amino acid residues in a selection of human immunoglobulin variable light chain (VL) or variable heavy chain (VH) framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup Ill as in Kabat et al., supra.

A “humanized” antibody refers to a chimeric antibody comprising amino acid residues from non-human CDRs and amino acid residues from human frameworks (FRs). At least a portion of a humanized antibody constant region is derived from a human antibody. In preferred embodiments of the bispecific antibody molecules disclosed herein, the two constant regions are from a human IgG antibody, such as, human IgG1. In preferred embodiments, the bispecific antibody molecules disclosed herein include a first and a second heavy chain each comprising a heavy chain variable region as provided herein and a human IgG1 constant region having the amino acid sequence sequence set forth in UniProt: P01857-1, version 1 modified by the knob-into-hole substitutions described herein. In preferred embodiments, the antibody molecules disclosed herein include a humanized light chain or a light chain comprising a variable light chain region as provided herein and a human light chain constant region. In preferred embodiments, the human light chain constant region is a human kappa light chain constant region. In certain aspects, the human IgG (IgG1) constant region additionally includes a KKDD enhancing heterodimer formation through electrostatic steering effects. In certain aspects, the human IgG1 heavy chain constant region present in the subject antibodies may include additional mutations, e.g., substitutions to modulate Fc function. For example, the LALAPG effector function mutations (L234A, L235A, and P329G) or the N297A mutation may be introduced to reduce antibody dependent cellular cytotoxicity (ADCC). The numbering of the substitutions is based on the EU numbering system. The “EU numbering system” or “EU index” is generally used when referring to a residue in an immunoglobulin heavy chain constant region (e.g., the EU index reported in Kabat et al., Sequences of Proteins of Immunological Interest, 5th Ed. Public Health Service, National Institutes of Health, Bethesda, MD. (1991)). The “EU index as in Kabat” refers to the residue numbering of the human IgG 1 EU antibody.

A “humanized form” of an antibody, e.g., a non-human antibody, refers to an antibody that has undergone humanization.

The term “epitope” refers to a region of an antigen that is recognized by the immune system, for example by antibodies, B cells, or T cells. For example, the epitope is the specific region of the antigen to which an antibody binds.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In some embodiments, the antibody will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated antibody includes the antibody in situ within recombinant cells since at least one component of the antibody's natural environment will not be present. In some instances, isolated antibody will be prepared by at least one purification step.

The multi-specific antibodies herein are generally purified to substantial homogeneity. The terms “substantial homogeneity,” “substantially homogeneous,” and “substantially homogeneous form” are used to indicate that the product is substantially devoid of by-products originated from undesired polypeptide combinations (e.g., homodimers or homo-multimers). Expressed in terms of purity, substantial homogeneity means that the amount of by-products does not exceed 10%, 9%, 8%, 7%, 6%, 4%, 3%, 2% or 1% by weight or is less than 1% by weight. In one embodiment, the by-product is below 5% by weight.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); single-chain antibody molecules, including antibodies comprising only heavy chains (e.g. VHH camelid antibodies); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂fragment that has two antigen combining sites and is still capable of cross-linking antigen.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Fv” is the minimum antibody fragment which contains a complete antigen-recognition and—binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen and can form the antigen binding site, although at a lower affinity than the entire binding site comprising the three CDRs of each variable domain.

The “Fab” fragment also contains the constant domain of the light chain and the first constant domain (CH₁) of the heavy chain. Fab fragments differ from Fab′ fragments by the addition of a few residues at the carboxyl terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Single-chain Fv”, “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L}domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “Fc region”, as used herein, generally refers to a dimer complex comprising the C-terminal polypeptide sequences of an immunoglobulin heavy chain, wherein a C-terminal polypeptide sequence is that which is obtainable by papain digestion of an intact antibody. The Fc region may comprise native or variant Fc sequences. Although the boundaries of the Fc sequence of an immunoglobulin heavy chain might vary, the human IgG heavy chain Fc sequence is usually defined to stretch from an amino acid residue at about position Cys226, or from about position Pro230, to the carboxyl terminus of the Fc sequence. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991. The Fc sequence of an immunoglobulin generally comprises two constant domains, a CH2 domain and a CH3 domain, and optionally comprises a CH4 domain. By “Fc polypeptide” herein is meant one of the polypeptides that make up an Fc region, e.g., a monomeric Fc. An Fc polypeptide may be obtained from any suitable immunoglobulin, such as IgG1, IgG2, IgG3, or IgG4 subtypes, IgA, IgE, IgD or IgM. The effector functions of antibodies are determined by sequences in the Fc region; this region is also the part recognized by Fc receptors (FcR) found on certain types of cells. In some embodiments, an Fc polypeptide comprises part or all of a wild-type hinge sequence (generally at its N terminus). In some embodiments, an Fc polypeptide does not comprise a functional or wild type hinge sequence.

A “native sequence Fc region” or “wild-type Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

In certain embodiments, the multi-specific antibody herein comprises an IgG Fc region, preferably derived from a wild-type human IgG1 Fc region. By “wild-type” human IgG Fc it is meant a sequence of amino acids that occurs naturally within the human population. The Fc sequence may vary slightly between individuals and such variations are still included within the definition of “Wild-type” human IgG Fc. For example, the Fc region may contain additional alterations that are not related to the present invention, such as a mutation in a glycosylation site or inclusion of an unnatural amino acid.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95%, at least about 96%, at least about 97%, at least about 98% or at least about 99% homology therewith.

The term “CH2 domain” of a human IgG Fc region usually extends from about residues 231 to about 340 of the IgG according to the EU numbering system. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain (Burton, Molec. Immunol. 22:161-206 (1985)).

The “knob-into-hole” or “K-i-H” technology directs the pairing of two polypeptides in vitro or in vivo by introducing a pertuberance (knob) into one polypeptide and a cavity (hole) into the other polypeptide at an interface in which they interact. For example, K-i-Hs have been introduced in the Fc:Fc binding interfaces, CL:CH1 interfaces or VH/VL interfaces of antibodies (see, e.g., Zhu et al., 1997 Protein Science 6:781-788; WO 96/027011, and WO 98/050431). This is especially useful in driving the pairing of two different heavy chains together during the manufacture of multispecific antibodies, such as bispecific antibodies (BsAb) (Carter, J. Immunol. Methods, 2001, 248:7-15 and Klein et al., 2012, MAbs 4(6):653-663), including bispecific IgG antibodies (BslgG). Multi-specific antibodies having K-i-H modifications in their Fc regions can further comprise single variable domains linked to each Fc region or can further comprise different heavy chain variable domains that pair with similar or different light chain variable domains. When used in conjunction with knob, hole or knob/hole, “wild-type” is meant to refer to the protein sequence without artificially introduced mutations, such as the knob, hole or knob/hole (K-i-H) mutations introduced herein, but otherwise to include all sequences that occur naturally within the human population.

A “protuberance” refers to at least one amino acid side chain which projects from the interface of a first polypeptide and is therefore positionable in a compensatory cavity in the adjacent interface (i.e. the interface of a second polypeptide) so as to stabilize the hetero-multimer, and thereby favor hetero-multimer formation over homo-multimer formation, for example. The protuberance may exist in the original interface or may be introduced synthetically (e.g. by altering nucleic acid encoding the interface). Normally, nucleic acid encoding the interface of the first polypeptide is altered to encode the protuberance. To achieve this, the nucleic acid encoding at least one “original” amino acid residue in the interface of the first polypeptide is replaced with nucleic acid encoding at least one “import” amino acid residue which has a larger side chain volume than the original amino acid residue. It will be appreciated that there can be more than one original and corresponding import residue. The upper limit for the number of original residues which are replaced is the total number of residues in the interface of the first polypeptide. The preferred import residues for the formation of a protuberance are generally naturally occurring amino acid residues and are preferably selected from arginine (R), phenylalanine (F), tyrosine (Y) and tryptophan (W). Most preferred are tryptophan and tyrosine. In one embodiment, the original residue for the formation of the protuberance has a small side chain volume, such as alanine, asparagine, aspartic acid, glycine, serine, threonine or valine.

A “cavity” refers to at least one amino acid side chain which is recessed from the interface of a second polypeptide and therefore accommodates a corresponding protuberance on the adjacent interface of a first polypeptide. The cavity may exist in the original interface or may be introduced synthetically (e.g., by altering nucleic acid encoding the interface). Normally, nucleic acid encoding the interface of the second polypeptide is altered to encode the cavity. To achieve this, the nucleic acid encoding at least one “original” amino acid residue in the interface of the second polypeptide is replaced with DNA encoding at least one “import” amino acid residue which has a smaller side chain volume than the original amino acid residue. It will be appreciated that there can be more than one original and corresponding import residue. The upper limit for the number of original residues which are replaced is the total number of residues in the interface of the second polypeptide. The preferred import residues for the formation of a cavity are usually naturally occurring amino acid residues and are preferably selected from alanine (A), serine (S), threonine (T) and valine (V). Most preferred are serine, alanine or threonine. In one embodiment, the original residue for the formation of the cavity has a large side chain volume, such as tyrosine, arginine, phenylalanine or tryptophan.

The protuberance or cavity can be “introduced” into the interface of a first or second polypeptide by synthetic means, e.g., by recombinant techniques, in vitro peptide synthesis, by enzymatic or chemical coupling of peptides or some combination of these and other techniques known in the art.

A “functional Fc region” possesses an “effector function” of a native sequence Fc region. Exemplary “effector functions” include C1q binding; CDC; Fc receptor binding; ADCC; phagocytosis; down regulation of cell surface receptors (e.g., B cell receptor; BCR), etc. Such effector functions generally require the Fc region to be combined with a binding domain (e.g., an antibody variable domain) and can be assessed using various assays as disclosed, for example, in definitions herein.

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. A MDR1-specific antibody binds specifically to an epitope within a MDR1 polypeptide. Non-specific binding would refer to binding with an affinity of less than about 10⁻⁷ M, e.g., binding with an affinity of 10⁻⁶ M, 10⁻⁵ M, 10⁻⁴ M, etc.

As used herein, the term “CDR” or “complementarity determining region” is intended to mean the non-contiguous antigen combining sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1
CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein, the term “framework” when used in reference to an antibody variable region is intended to mean all amino acid residues outside the CDR regions within the variable region of an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by the CDRs. A VH chain can comprise three CDRs and four FRs arranged from N-terminus to C-terminus in the following order: FR1, CDR1, FR2, CDR2, FR3, CDR3, FR4. Similarly, a VL chain can comprise three CDRs and four FRs arranged from N-terminus to C-terminus in the following order: FR1, CDR1, FR2, CDR2, FR3, CDR3, FR4.

The term “cytotoxic agent” as used herein refers to a substance that inhibits or prevents a cellular function and/or causes cell death or destruction. A “chemotherapeutic agent,” also referred to an “antineoplastic agent,” can be a cytotoxic agent which is used for treating a cancer or other disease or disorder.

The terms “cancer” and “cancerous” refer to a physiological condition in mammals, including humans, that is typically characterized by unregulated cell growth/proliferation. Included in this definition are benign and malignant cancers. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastric or stomach cancer including gastrointestinal cancer, pancreatic cancer, glioblastoma, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer (e.g., renal cell carcinoma), liver cancer, prostate cancer, vulval cancer, thyroid cancer, hepatic carcinoma, anal carcinoma, penile carcinoma, melanoma, and various types of head and neck cancer.

By “early-stage cancer” is meant a cancer that is not invasive or metastatic or is classified as a Stage 0, I, or II cancer.

The term “precancerous” refers to a condition or a growth that typically precedes or develops into a cancer.

By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage Ill cancer.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, including in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” refers to the amount of a target-specific antibody that, when administered to a mammal or other subject for treating a disease, is sufficient to affect such treatment for the disease. The “therapeutically effective amount” will vary depending on the antibody, the disease and its severity and the age, weight, etc., of the subject to be treated. The therapeutically effective amount of a multi-specific humanized antibody herein may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the antibody or antibody fragment may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life. “Reduce” or “inhibit” can refer to the symptoms of the disorder being treated, the presence or size of metastases, the size of the primary tumor, or the size or number of the blood vessels in angiogenic disorders.

The term “refractory”, used herein, refers to a disease or condition that does not respond to treatment. With regard to cancer, “refractory cancer”, as used herein, refers to cancer that does not respond to treatment. A refractory cancer may be resistant at the beginning of treatment or it may become resistant during treatment. Refractory cancer may also be called resistant cancer.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, serum, plasma, biological fluid, and tissue samples.

Percent identity between a pair of sequences may be calculated by multiplying the number of matches in the pair by 100 and dividing by the length of the aligned region, including gaps. Identity scoring only counts perfect matches and does not consider the degree of similarity of amino acids to one another. Only internal gaps are included in the length, not gaps at the sequence ends. Percent Identity=(Matches x 100)/Length of aligned region (with gaps)

The phrase “conservative amino acid substitution” refers to substitution of amino acid residues within the following groups: 1) L, I, M, V, F; 2)R, K; 3) F, Y, H, W, R; 4) G, A, T, S; 5) Q, N; and 6) D, E. Conservative amino acid substitutions may preserve the activity of the protein by replacing an amino acid(s) in the protein with an amino acid with a side chain of similar acidity, basicity, charge, polarity, or size of the side chain.

The term “vector” means any molecule or entity (e.g., nucleic acid, plasmid, bacteriophage or virus) used to transfer protein coding information into a host cell.

The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct may include, but is not limited to, sequences that affect or control transcription, translation, and, if introns are present, affect RNA splicing of a coding region operably linked thereto.

The term “stimulation,” refers to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

The term “stimulatory molecule,” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In one aspect, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing cytoplasmic signaling sequence that is of particular use in the invention includes, but is not limited to, those derived from CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12.

The term a “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to, an MHC class I molecule, BTLA and a Toll ligand receptor, as well as OX40, CD27, CD28, CDS, ICAM-1, LFA-1 (CD11a/CD18), ICOS (CD278), and 4-1 BB (CD137).

The term “autologous” refers to any material derived from the same individual to whom it is later to be re-introduced into the individual.

An “intracellular signaling domain,” as the term is used herein, refers to an intracellular portion of a molecule. The intracellular signaling domain generates a signal that promotes an immune effector function of the CAR containing cell, e.g., a CAR-T cell. Examples of immune effector function, e.g., in a CAR-T cell, include cytolytic activity and helper activity, including the secretion of cytokines.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes.

Guidance for substitutions, insertions, or deletions may be based on alignments of amino acid sequences of proteins from different species or from a consensus sequence based on a plurality of proteins having the same or similar function.

### DETAILED DESCRIPTION

Provided are multi-specific humanized anti-ABCB1 antibodies that target both ABCB1 and a tumor-associated antigen (TAA) as well as pharmaceutical compositions, nucleic acids, recombinant expression vectors, cells, and kits that include or encode such multi-specific antibodies. In some embodiments, the multi-specific antibodies include a common variable light (VL) chain that includes an antigen-binding site for ABCB1, a first variable heavy (VH) chain that includes an antigen-binding site for ABCB1, and a second VH chain that includes an antigen-binding site for the TAA. Methods and uses for treating conditions where inhibition of ABCB1-mediated influx is desirable, such as methods and uses for treating cancer are also included. Such methods include administering to the subject a multi-specific humanized anti-ABCB1 antibody that targets both ABCB1 and a TAA as described and claimed herein. The treating may involve administering the multi-specific antibody alone or administering the multi-specific antibody and a chemotherapeutic agent. Further provided are methods of generating the described multi-specific antibodies and reagents related thereto, including genetically modified cell lines useful in the subject methods and methods of making such genetically modified cell lines.

The multi-specific (e.g., bispecific) antibodies provided herein bind to cancer cells expressing both ABCB1 and the TAA while showing reduced binding to non-cancer cells expressing ABCB1 and/or the TAA. In other words, the bispecific antibodies provided herein bind with low affinity to (1) cells expressing TAA where ABCB1 expression is low or absent and (2) cells expressing ABCB1 where TAA expression is low or absent, and with high affinity to cancer cells that express at least one or both ABCB1 and CD47 at relatively high levels, i.e., levels higher than normal cells.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the methods and compositions have or will be described for the sake of grammatical fluidity with functional explanations, it is to be expressly understood that the claims, unless expressly formulated under 35 U.S.C. § 112(f), are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112(f) are to be accorded full statutory equivalents under 35 U.S.C. § 112(f).

### Multi-Specific (Bispecific) Antibodies

To generate a substantially homogeneous population of heterodimeric polypeptides, such as multi-specific or bispecific antibodies, the process must have a strong preference for forming heterodimers over homodimers. A well-known approach to address this challenge is the use of the so-called knobs-into-holes technique, which is based on engineering the Fc region of antibody heavy chains as described, for example, in Carter, P., Journal of Immunological Methods 248 (2001) 7-15; and U.S. Pat. Nos. 5,731,168 and 7,642,228. In brief, the knobs-into-holes approach provides that in a bispecific antibody the Fc regions of the first and the second heavy chains meet at an interface, where the interface of one of the Fc regions comprises a protuberance (knob) that is positionable in a cavity (hole) in the interface of the other Fc region. The protuberance and the cavity may be created by introducing alterations, such as substitutions, in one or both of the Fc region sequences. Thus, a protuberance may be generated by replacing an original residue in the Fc sequence with an import residue having a larger side-chain volume than the original residue.

In certain aspects, the present disclosure provides multi-specific, such as bispecific, humanized IgG (IgG1) antibody molecules that target both multidrug resistance protein 1 (MDR1, ABCB1) and a tumor-associated antigen (TAA) and include matching knobs-into-holes (K-i-H) mutations in two IgG (IgG1) heavy chain Fc regions in combination with one or more additional mutations (substitutions). Such combination of amino acid alterations, in some instances combined with participating residues present in the native IgG (IgG1) heavy chain, promote proper orientation of the heavy chains relative to each other, improving overall yield and/or purity and/or homogeneity of the desired bispecific antibodies not only relative to corresponding bispecific antibodies without mutations in their Fc regions but generally also relative to bispecific antibodies comprising only traditional knobs-into-hole mutations.

Specifically, in this aspect, such multi-specific IgG1 antibody molecules comprise a first heavy chain that comprises an antigen-binding site for ABCB1 and a second heavy chain that comprises an antigen-binding site for a TAA each heavy chain comprising an Fc region, wherein one of the Fc regions comprises hole amino acid substitutions Y349C, T366S, L368A, and Y407V, in combination with amino acid substitution F405V or Q347R, wherein numbering is according to the EU numbering scheme. In such multi-specific IgG1 antibody molecules the other Fc region may comprise knob amino acid substitutions S354C and T366W, and optionally further comprises one or both of amino acid substitutions K392D and K409D, wherein numbering is according to the EU numbering scheme.

In some of the multi-specific IgG1 antibody molecules herein one of the Fc regions comprises hole amino acid substitutions Y349C, T366S, L368A, and Y407V in combination with substitutions (i) F405V; (ii) F405V, K409D; (iii) K409D; (iv) D399K; (v) F405V, D399K; or (vi) Q347R, wherein numbering is according to the EU numbering scheme.

In some of such multi-specific IgG1 antibody molecules the other Fc region comprises knob amino acid substitutions (i) S354C, T366W; (ii) S354C, T366W, D399K; (iii) S354C, T366W, K409D; or (iv) S354C, T366W, K360E, wherein numbering is according to the EU numbering scheme.

In one embodiment, in the subject multi-specific IgG1 antibody molecule one of the Fc regions comprises a set of amino acid substitutions and amino acid residues selected from the group consisting of:
(a) Y349C, T366S, L368A, Y407V, E356, E357, F405V;
(b) Y349C, T366S, L368A, Y407V, E356, E357, F405V, K409D;
(c) Y349C, T366S, L368A, Y407V, E356, E357, K409D;
(d) Y349C, T366S, L368A, Y407V, E356, E357, D399K;
(e) Y349C, T366S, L368A, Y407V, E356, E357, F405V, D399K; and
(f) Y349C, T366S, L368A, Y407V, E356, E357, Q347R, wherein numbering is according to the EU numbering scheme.

In another embodiment, the other Fc region comprises a set of amino acid substitutions and amino acid residues selected from the group consisting of:
(a) S354C, T366W, E356, E357;
(b) S354C, T366W, E356, E357, D399K;
(c) S354C, T366W, E356, E357, K409D;
(d) S354C, T366W, E356, E357, K360E,

wherein numbering is according to the EU numbering scheme.

Specifically included herein is a multi-specific IgG1 antibody molecule, wherein one of the Fc regions comprises the set of amino acid substitutions and amino acid residues Y349C, T366S, L368A, Y407V, E356, E357, F405V, and the other Fc region comprises the set of amino acid substitutions and amino acid residues S354C, T366W, E356, E357, wherein numbering is according to the EU numbering scheme.

In another group of multi-specific IgG1 antibody molecules, one of the Fc regions comprises the set of amino acid substitutions and amino acid residues Y349C, T366S, L368A, Y407V, E356, E357, K409D, and the other Fc region comprises the set of amino acid substitutions and amino acid residues S354C, T366W, E356, E357, D399K, wherein numbering is according to the EU numbering scheme.

Exemplary combinations of the substitutions that may be present in the heavy chain Fc regions of the subject ABCB1 X TAA multi-specific antibodies, along with participating residues that are present in the native Fc sequence, are shown in the following Table 2:

TABLE 2

| | |
|---|---|
| HIR1 | FC hIgG1 Y349C, T366S, L368A, Y407V, E356 E357 |
| KbR1 | FC huIgG1 S354C, T366W, E356 E357 |
| HIR2 | FC hIgG1 Y349C, T366S, L368A, Y407V, E356 E357 F405V |
| HIR3 | FC hIgG1 Y349C, T366S, L368A, Y407V, E356 E357 F405V |
| | K409D |
| KbR3 | FC huIgG1 S354C, T366W, E356 E357 D399K |
| HIR4 | FC hIgG1 Y349C, T366S, L368A, Y407V, E356 E357 K409D |
| HIR5 | FC hIgG1 Y349C, T366S, L368A, Y407V, E356 E357 D399K |
| KbR5 | FC huIgG1 S354C, T366W, E356 E357 K409D |
| HIR6 | FC hIgG1 Y349C, T366S, L368A, Y407V, E356 E357 F405V |
| | D399K |
| HIR7 | FC hIgG1 Y349C, T366S, L368A, Y407V, E356 E357 Q347R |
| KbR7 | FC huIgG1 S354C, T366W, E356 E357 K360E |

Any of the KbR1, KbR3, KbR5, and KbR7 sets of knob sites can be combined with any of the HIR1-HIR7 sets of hole sites. In one embodiment, the bispecific antibodies herein comprise HIR1/KbR3, HIR1/KbR5, HIR1/KbR7, HIR2/KbR1, HIR3/KbR4, HIR4/KbR3, HIR6/KbR5, or HIR7/KbR7 combinations. In another embodiment, the bispecific antibodies herein comprise the HIR2/KbR1 and HIR4/KbR3 combinations, especially the HIR2/KbR1 combination.

In certain embodiments, one IgG (IgG1) heavy chain of the bispecific anti-ABCB1 antibodies herein contain charge pair substitutions at the CH3 domain, thus one of the heavy chains may contain K392D and/or K409D substitutions and the other E356K and/or D399K substitutions to further facilitate assembly.

Any of the listed sets of amino acid substitutions can be present in either the heavy chain with binding specificity for ABCB1 or in the heavy chain binding to the TAA. Similarly, the “KK” and “DD” substitution, respectively can be present either in the ABCB1-binding heavy chain or in the heavy chain binding the TAA.

As discussed earlier, while any of the KbR1, KbR3, KbR5, and KbR7 sets can be combined with any of the HIR-HIR7 sets, the HIR1/KbR1, HIR2/KbR1, HIR4/KbR3, HIR3/KBR4, HIR6/KbR5, and HIR7/KbR7 combinations are preferred. Particularly preferred are the HIR2/KbR1 (format 1) and HIR4/KbR3 (format 2) combinations.

The format 1 and format 2 Fc polypeptide sequences with mutations relative to the wild-type human IgG1 Fc reference sequence, are shown below. The mutated residues and, in the wild-type sequence, the native residues participating in interactions facilitating pairing, are shown in bold.

Format 1
HC1 (hG1-HIR2):
(SEQ ID NO: 12)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVCTLPPSREEMTKNQVSLSC
AVKGFYPSDIAVEWESNGQPENNYKTTPPVLDSDGSFVLVSKLTVDKSRW
QQGNVFSCSVMHEALHNHYTQKSLSLSPGK
HC1 (hG1-KbR1):
(SEQ ID NO: 22)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPCREEMTKNQVSLWC
LVKGFYPSDIAVEWESNGQPENNYKTTPPVLDSDGSFFLYSKLTVDKSRW
QQGNVFSCSVMHEALHNHYTQKSLSLSPGK
Format 2
HC1 (hG1-HIR4):
(SEQ ID NO: 16)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVCTLPPSREEMTKNQVSLSC
AVKGFYPSDIAVEWESNGQPENNYKTTPPVLDSDGSFFLVSDLTVDKSRW
QQGNVFSCSVMHEALHNHYTQKSLSLSPGK
HC2 (hG1-KbR3):
(SEQ ID NO: 23)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPCREEMTKNQVSLWC
LVKGFYPSDIAVEWESNGQPENNYKTTPPVLKSDGSFFLYSKLTVDKSRW
QQGNVFSCSVMHEALHNHYTQKSLSLSPGK
human HC CHIg-hG1 reference sequence:
(SEQ ID NO: 42)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In another aspect, bispecific humanized IgG1 antibody molecules are provided that bind ABCB1 and a TAA and comprise two identical light chain variable regions, a first heavy chain variable region, and a second heavy chain variable region, wherein the light chain variable regions each comprise an antigen-binding site for ABCB1, the first heavy chain variable region comprises an antigen-binding site for ABCB1, the second heavy chain variable region comprises an antigen-binding site for the TAA, and the second VH chain binds the TAA when paired with one of the VL chains, and wherein the antigen-binding site of the two identical light chain variable regions comprises light chain CDRs 1-3 (LCDRs 1-3) of a light chain variable region sequence selected from the group consisting of:

(SEQ ID NO: 1)
DIVMTQTPLSSPVTLGQPASISCRSSQSIVHSTGNTYLEWYQQRPGQPPR
LLIYKISNRFSGVPDRFSGSGAGTDFTLKISRVEAEDVGVYYCFQASHFP
RTFGGGTKLEIKR (MRK16 hmz);
(SEQ ID NO: 2)
DIVMTQSPLSLPVTPGEPASISCRSSQSLVHSNGNTYLEWYLQKPGQSPQ
LLIYKVSNRFSGVPDRFSGSGSGTDFTLKISRVEAEDVGVYYCFQGSHFP
RTFGQGTKLEIK (B1VL6/CDR3v2a hmz);
(SEQ ID NO: 3)
DIVMTQTPLSSPVTLGQPASISCRSSQSIVHSTGNTYLEWYQQKPGQPPR
LLIYKISNRFSGVPDRFSGSGSGTDFTLKISRVEAEDVGVYYCFQASHFP
RTFGQGTKLEIK (MRK16v4a1 hmz);
(SEQ ID NO: 4)
DIVMTQTPLSSPVTLGQPASISCRSSQSIVHSTGNTYLEWYQQKPGQPPR
LLIYKVSNRFSGVPDRFSGSGSGTDFTLKISRVEAEDVGVYYCFQASHFP
RTFGQGTKLEIK (MRK16v4a12 hmz);
(SEQ ID NO: 6)
DIVMTQSPLSLPVTPGEPASISCRSSQTIVHSNGNTYLEWYLQKPGQSPQ
LLIYKVSKRFSGVPDRFSGSGSGTDFTLKISRVEAEDVGVYYCFQASHFP
RTFGQGTKLEIK (B1.89v1.huL1 (KV2) hmz);
(SEQ ID NO: 7)
DVVLTQSPLSLPVTPGEPASISCRSSQNIVHSTGNTYLDWYLQKPGQSPQ
LLIYKVSNRFSGVPDRFSGSGSGTDFTLKISRVEAEDVGVYYCFQGSHIP
RTFGQGTKLEIKR (B2.28.huL2);
and
(SEQ ID NO: 8)
DIVMTQSPLSLPVSLGDPASISCRSSQSLVHSNGNTYLEYYLQKPGQSPK
LLIYKVSNRFSGVPDRFSGSGSGTDFTLKISRVEAEDLGVYYCFQGSHFP
RTFGGGTKLEIK (B1VL6/CDR3v2 hmz),

wherein LCDRs 1-3 are shown in bold.

In one embodiment, the bispecific humanized IgG1 antibody molecule comprising a common humanized light chain, having a variable region selected from the humanized variable regions listed above.

In some embodiments, the subject bispecific humanized IgG1 molecules comprise any of the above-described combinations of Fc mutations.

Anti-ABCB1 and Anti-CD47 (ABCB1 X CD47) Multi-Specific Antibodies In certain aspects, a multi-specific antibody molecule herein binds to ABCB1 and CD47 and comprises any of the combinations of amino acid substitutions described above in its Fc region. Representative sequences of the first and second heavy chains of such antibodies, Fc sequences with matching HIR and KbR mutations, and VH and VL sequences, including CDRs, of such antibodies are presented in Table 3 below. However, such sequences are for illustration only and are not limiting. All multi-specific antibodies that bind ABCB1 and CD47 and comprise any of the mutations listed above in any combination are specifically included.

The ABCB1-binding domain and CD47-binding domains in such antibodies may vary, including the epitopes bound by the domains, the variable region arrangement and sequences, and other similar factors.

A subject ABCB1-binding domain specifically binds one or more epitopes of ABCB1. Thus, the epitope is an ABCB1 epitope. The size of an ABCB1 epitope bound by a ABCB1-binding domain may vary, including where the ABCB1 epitope is formed by a polypeptide having a contiguous stretch of a ABCB1 sequence that may range from 4 aa or less to 12 aa or more, including but not limited to e.g., 4 aa, 5 aa, 6 aa, 7 aa, 8 aa, 9 aa, 10 aa, 11 aa, 12 aa, 4 aa to 10 aa, 5 aa to 10 aa, 6 aa to 10 aa, 4 aa to 8 aa, 5 aa to 8 aa, 6 aa to 8 aa, etc.

A subject ABCB1-binding domain exhibits high affinity binding to ABCB1. For example, a subject MDR1-binding domain binds to MDR1 with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M. A subject MDR1-binding domain binds to an epitope present on MDR1with an affinity of from about 10⁻⁷ μM to about 10⁻⁸ M, from about 10⁻⁸ μM to about 10⁻⁹ M, from about 10⁻⁹ μM to about 10⁻¹⁰ M, from about 10⁻¹⁰ μM to about 10⁻¹¹ M, or from about 10⁻¹¹ μM to about 10⁻¹² M, or greater than 10⁻¹² M.

A subject ABCB1-binding domain exhibits substantially no binding to any epitopes formed by amino acids within other related, but sequence dissimilar, proteins such as related but sequence dissimilar EPs. Any binding of a subject ABCB1-binding domain to an epitope formed by amino acids within a related, but sequence dissimilar, protein is generally non-specific binding of a substantially lower affinity than the specific binding of the ABCB1-binding domain to the epitope on ABCB1. A substantially lower affinity is generally at least a two-fold, three-fold, five-fold, 10-fold, 50-fold, 100-fold, 500-fold, or 1000-fold lower affinity.

A subject ABCB1-binding domain can reduce transport of molecules through a ABCB1 transporter. For example, a subject ABCB1-binding domain can reduce transport by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the degree of transport in the absence of the ABCB1-binding domain.

In addition to the combinations of mutations described herein, including charge to swap mutations (e.g., E356K, D399K/K392D, K409D mutations), the Fc region can be further modified, including modifications that reduce or abrogate binding of the antibody to one or more Fcγ receptors. In certain aspects, the IgG1 Fc domain may have one or more of the substitutions L234A, L235A, P329G and N297A/Q/G.

In some embodiments, a subject ABCB1XCD47 multi-specific antibody comprises a humanized heavy chain, binding to ABCB1, that comprises one of the heavy chain variable region sequences listed below, or CDRs 1-3 (HCDRs 1-3) of one of such heavy chain variable region sequences:

(SEQ ID NO: 9)
EVQLVESGGVVVQPGGSLRLSCAASGFTFSRYTMSWVRQAPGKGLEWVAT
ISSGGGNTYYPDSVKGRFTVSRDNSKNSLYLQMNSLRTEDTALYYCARYG
AGDAWFAYWGQGTLVTVSS (15D3 hmz);
(SEQ ID NO: 10)
EVQLVESGGVVVQPGGSLRLSCAASGFTFSRYTMSWVRQAPGKGLEWVAT
ISSGGGNTYYPDSVKGRFTVSRDNSKNSLYLQMNSLRTEDTALYYCARYY
RYDAWFAYWGQGTLVTVSS (15D3hmzv16-1 hG1);
(SEQ ID NO: 43)
EVQLVESGGVVVQPGGSLRLSCAASGFTFSRYTMSWVRQAPGKGLEWVAT
ISSGGGNTYYPDSVKGRFTVSRDNSKNSLYLQMNSLRTEDTALYYCARYG
RGDAWFAYWGQGTLVTVSSASTK (15D3hmzv16-2 hG1);
(SEQ ID NO: 44)
EVQLVESGGVVVQPGGSLRLSCAASGFTFSRYTMSWVRQAPGKGLEWVAT
ISSGGGNTYYPDSVKGRFTVSRDNSKNSLYLQMNSLRTEDTALYYCARYY
AYDAWFAYWGQGTLVTVSSASTK (15D3hmzv16-3 hG1);
(SEQ ID NO: 45)
EVQLVESGGVVVQPGGSLRLSCAASGFTFSRYTMSWVRQAPGKGLEWVAT
ISSGGGNTYYPDSVKGRFTVSRDNSKNSLYLQMNSLRTEDTALYYCARYG
AGDAWFASWGQGTLVTVSSASTK (15D3hmzv16-4 hG1);
and
(SEQ ID NO: 46)
EVQLVESGGVVVQPGGSLRLSCAASGFTFSRYTMSWVRQAPGKGLEWVAT
ISSGGGNTYYPDSVKGRFTVSRDNSKNSLYLQMNSLRTEDTALYYCARYY
AYDAWFASWGQGTLVTVSSASTK (15D3hmzv16-5 hG1).

In some embodiments, an ABCB1XCD47 bispecific antibody, comprises a humanized heavy chain, binding to ABCB1, comprising one of the following heavy chain sequences:

(SEQ ID NO: 31)
EVQLVESGGVVVQPGGSLRLSCAASGFTFSRYTMSWVRQAPGKGLEWVAT
ISSGGGNTYYPDSVKGRFTVSRDNSKNSLYLQMNSLRTEDTALYYCARYG
AGDAWFAYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYDTTPPVL
DSDGSFFLYSDLTVDKSRWQQGNVFSCSVMHEALHNHYTQKSLSLSPG
(15D3 hmz DD huIgG1 HC);
(SEQ ID NO: 33)
EVQLVESGGVVVQPGGSLRLSCAASGFTFSRYTMSWVRQAPGKGLEWVAT
ISSGGGNTYYPDSVKGRFTVSRDNSKNSLYLQMNSLRTEDTALYYCARYG
AGDAWFAYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
CTLPPSREEMTKNQVSLSCAVKGFYPSDIAVEWESNGQPENNYKTTPPVL
DSDGSFVLVSKLTVDKSRWQQGNVFSCSVMHEALHNHYTQKSLSLSPG
(15D3 hmz HIR2 huIgG1 HC);
and
(SEQ ID NO: 34)
EVQLVESGGVVVQPGGSLRLSCAASGFTFSRYTMSWVRQAPGKGLEWVAT
ISSGGGNTYYPDSVKGRFTVSRDNSKNSLYLQMNSLRTEDTALYYCARYG
AGDAWFAYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
CTLPPSREEMTKNQVSLSCAVKGFYPSDIAVEWESNGQPENNYKTTPPVL
DSDGSFFLVSDLTVDKSRWQQGNVFSCSVMHEALHNHYTQKSLSLSPG
(15D3 hmz HIR4 huIgG1 HC).

In some embodiments, the heavy chain sequence can be extended at the C-terminus by addition of one or more amino acids. In some embodiments, the heavy chain sequence set forth in SEQ ID NOs:33 and 34 may be extended by one amino acid, where the one amino acid is K.

In some embodiments, an ABCB1XCD47 bispecific antibody comprises a second heavy chain, which binds CD47 and comprises the variable region, shown below, or CDRs1-3 (HCDRs1-3) of such variable region, shown in bold:

(SEQ ID NO: 11)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSS (KT14 aCD47).

In some embodiments, an ABCB1XCD47 bispecific antibody, comprises one of the following second heavy chains, binding to CD47:

(SEQ ID NO: 32)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRKELTKNQVSLTCLVKGFYPSDIAVEWESNGQPENNYKTTPPVLKS
DGSFFLYSKLTVDKSRWQQGNVFSCSVMHEALHNHYTQKSLSLSPG
(KT14 KK huIgG1 HC);
(SEQ ID NO: 35)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVCT
LPPSREEMTKNQVSLSCAVKGFYPSDIAVEWESNGQPENNYKTTPPVLDS
DGSFFLVSKLTVDKSRWQQGNVFSCSVMHEALHNHYTQKSLSLSPGK
(KT14 HIR1 huIgG1 HC);
(SEQ ID NO: 36)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVCT
LPPSREEMTKNQVSLSCAVKGFYPSDIAVEWESNGQPENNYKTTPPVLDS
DGSFVLVSDLTVDKSRWQQGNVFSCSVMHEALHNHYTQKSLSLSPGK
(KT14 HIR3 huIgG1 HC);
(SEQ ID NO: 120)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPCREEMTKNQVSLWCLVKGFYPSDIAVEWESNGQPENNYKTTPPVLDS
DGSFFLYSKLTVDKSRWQQGNVFSCSVMHEALHNHYTQKSLSLSPG
(KT14 KbR1 huIgG1 HC);
and
(SEQ ID NO: 121)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPCREEMTKNQVSLWCLVKGFYPSDIAVEWESNGQPENNYKTTPPVLKS
DGSFFLYSKLTVDKSRWQQGNVFSCSVMHEALHNHYTQKSLSLSPG
(KT14 KbR3 huIgG1 HC).

In another aspect, the subject ABCB1XCD47 bispecific antibody comprises a humanized light chain binding ABCB1, that comprises one of the light chain variable region sequences listed below, or CDRs 1-3 (LODRs 1-3) of one of such light chain variable region sequences, where such CDRs are shown in bold:

(SEQ ID NO: 1)
DIVMTQTPLSSPVTLGQPASISCRSSQSIVHSTGNTYLEWYQQRPGQPPR
LLIYKISNRFSGVPDRFSGSGAGTDFTLKISRVEAEDVGVYYCFQASHFP
RTFGGGTKLEIKR (MRK16 hmz);
(SEQ ID NO: 2)
DIVMTQSPLSLPVTPGEPASISCRSSQSLVHSNGNTYLEWYLQKPGQSPQ
LLIYKVSNRFSGVPDRFSGSGSGTDFTLKISRVEAEDVGVYYCFQGSHFP
RTFGQGTKLEIK (B1VL6/CDR3v2a hmz);
(SEQ ID NO: 3)
DIVMTQTPLSSPVTLGQPASISCRSSQSIVHSTGNTYLEWYQQKPGQPPR
LLIYKISNRFSGVPDRFSGSGSGTDFTLKISRVEAEDVGVYYCFQASHFP
RTFGQGTKLEIK (MRK16v4a1 hmz);
(SEQ ID NO: 4)
DIVMTQTPLSSPVTLGQPASISCRSSQSIVHSTGNTYLEWYQQKPGQPPR
LLIYKVSNRFSGVPDRFSGSGSGTDFTLKISRVEAEDVGVYYCFQASHFP
RTFGQGTKLEIK (MRK16v4a12 hmz);
(SEQ ID NO: 6)
DIVMTQSPLSLPVTPGEPASISCRSSQTIVHSNGNTYLEWYLQKPGQSPQ
LLIYKVSKRFSGVPDRFSGSGSGTDFTLKISRVEAEDVGVYYCFQASHFP
RTFGQGTKLEIK (B1.89v1.huL1 (KV2) hmz);
(SEQ ID NO: 7)
DVVLTQSPLSLPVTPGEPASISCRSSQNIVHSTGNTYLDWYLQKPGQSPQ
LLIYKVSNRFSGVPDRFSGSGSGTDFTLKISRVEAEDVGVYYCFQGSHIP
RTFGQGTKLEIKR (B2.28.huL2);
and
(SEQ ID NO: 8)
DIVMTQSPLSLPVSLGDPASISCRSSQSLVHSNGNTYLEYYLQKPGQSPK
LLIYKVSNRFSGVPDRFSGSGSGTDFTLKISRVEAEDLGVYYCFQGSHFP
RTFGGGTKLEIK (B1VL6/CDR3v2 hmz),

In some embodiments, a subject ABCB1XCD47 bispecific antibody comprises one of the following humanized light chains, binding ABCB1:

(SEQ ID NO: 38)
DVLMTQTPVSLSVSLGDQASISCRSSQSIVHSTGNTYLEWYLQKPGQSPK
LLIYKISNRFSGVPDRFSGSGSGTDFTLKISRVEAEDLGVYYCFQASHFP
RTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC (MRK16 LC);
(SEQ ID NO: 37)
DIVMTQSPLSLPVTPGEPASISCRSSQTIVHSNGNTYLEWYLQKPGQSPQ
LLIYKVSKRFSGVPDRFSGSGSGTDFTLKISRVEAEDVGVYYCFQASHFP
RTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC (B1.89v1.huL1 (KV2) LC);
and
(SEQ ID NO: 39)
DIVMTQSPLSLPVTPGEPASISCRSSQSLVHSNGNTYLEWYLQKPGQSPQ
LLIYKVSNRFSGVPDRFSGSGSGTDFTLKISRVEAEDVGVYYCFQGSHFP
RTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC (B1VL6/CDR3v2a LC).

Such humanized ABCB1XCD47 bispecific antibodies may comprise any two heavy chains comprising ABCB1—and CD47-binding domains, respectively, with or without the Fc mutations discussed and listed above. All bispecific antibodies that comprise any of the humanized light chain variable domains set forth above are specifically included herein, as are the humanized light chain molecules. As discussed above, the ABCB1-binding domain and CD47-binding domains in such antibodies may vary, including the epitopes bound by the domains, the variable region arrangement and sequences, and other similar factors.

In addition to the described humanized light chains, the first VH chain, and or the second VH may be humanized. Thus, bispecific ABCB1XCD47 antibodies comprising a common humanized light chain as described above, may comprise one of the humanized heavy chain variable domains disclosed above, and may have Fc mutations as hereinabove discussed.

In certain aspects, the humanized IgG bispecific antibody molecule specifically binds a cell expressing both ABCB1 and CD47 and has greater than twice the affinity for a cell expressing both ABCB1 and the CD47 as compared to a cell expressing either ABCB1 or CD47.

In certain aspects, the bispecific antibody molecule is capable of increasing sensitivity of a cancer cell to treatment with a chemotherapeutic agent, where the half maximal inhibitory concentration (IC50) of the chemotherapeutic agent when co-administered with the antibody is at least 2 times lower (e.g. at least 3 times lower, at least 4 times lower, at least 5 times lower, at least 10 times lower, at least 20 times lower, or at least 30 times lower) than the IC50 of the chemotherapeutic agent when co-administered with an anti-ABCB1 antibody comprising a VH chain having the sequence:
EVKVVESGGVLVRPGGSLKLSCAASGFTFSRYTMSWVRQTPEKRLEWVATISSGGGNTYYPD SVKGRFTVSRDNAMSSLYLQMSSLRSEDTALYYCARYGAGDAWFAYWGQGTLVTVSA (SEQ ID NO:47); and a VL chain having the sequence:
DVLMTQTPLSLPVSLGDQASISCRSSQSIVHSTGNTYLEWYLQKPGQSPKLLIYKVSNRFSGVP DRFSGSGSGTDFTLKISRLEAEDLGVYYCFQGSHFPRTFGGGTRLEIK (SEQ ID NO:48). In certain aspects, the anti-MDR1 antibody may be the 15D3 antibody described in U.S. Pat. No. 5,959,084.

In certain aspects, the bispecific antibody molecule binds to ABCB1 with at least 2-fold lower affinity (e.g. at least 3 times lower, at least 4 times lower, at least 5 times lower, at least 10 times lower, at least 20 times lower, or at least 30 times lower affinity) than an anti-MDR1 antibody comprising a VH chain having the sequence:
EVKVVESGGVLVRPGGSLKLSCAASGFTFSRYTMSWVRQTPEKRLEWVATISSGGGNTYYPD SVKGRFTVSRDNAMSSLYLQMSSLRSEDTALYYCARYGAGDAWFAYWGQGTLVTVSA (SEQ ID NO:47); and a VL chain having the sequence:
DVLMTQTPLSLPVSLGDQASISCRSSQSIVHSTGNTYLEWYLQKPGQSPKLLIYKVSNRFSGVP DRFSGSGSGTDFTLKISRLEAEDLGVYYCFQGSHFPRTFGGGTRLEIK (SEQ ID NO:48). In certain aspects, the anti-ABCB1 antibody may the 15D3 antibody described in U.S. Pat. No. 5,959,084.

In certain aspects, the bispecific antibody molecule when bound to a cell expressing a ABCB1, inhibits efflux by the ABCB1. Inhibition may be a decrease in efflux by at least 5%, at least 10%, at least 15%, at least 20%, at least 25%, at least 50%, or more, as compared to efflux by the MDR1 in absence of the bispecific antibody.

As summarized above, the present disclosure provides multi-specific antibodies having a domain that targets a cellular efflux pump and a domain that targets a cancer-associated antigen. Included are multi-specific antibodies that include a multidrug resistance protein 1 (ABCB1)-binding domain and a leukocyte surface antigen CD47-binding domain. Multi-specific antibodies of the present disclosure specifically bind cells that express both ABCB1 and CD47.

Accordingly, the multi-specific antibodies of the present disclosure target both ABCB1 and CD47. ABCB1 (MDR1, also known as P-glycoprotein 1 (Pgp)), is an energy-dependent efflux pump responsible for decreased drug accumulation in multidrug-resistant cells that is expressed from the ATP binding cassette subfamily B member 1 (ABCB1). CD47, also known as integrin associated protein (IAP), is an immunoglobulin superfamily transmembrane protein that binds membrane integrins and also serves as a receptor for the ligands thrombospondin-1 (TSP-1) and signal-regulatory protein alpha (SIRPα) and is encoded by the CD47 gene. CD47 ligand binding can result in inhibition of phagocytosis and thus, as a target in immune therapy, masking of the CD47 extracellular domain prevents inhibition of immune-mediated killing of CD47-expressing cancer cells.

Thus, multi-specific antibodies of the present disclosure bind cells that express both ABCB1 and CD47 with higher affinity than cells that express only ABCB1 or CD47. Correspondingly, multi-specific antibodies of the present disclosure bind with much reduced affinity when low levels of the respective second target are present, e.g., as compared to when both first and second targets are present above low levels (e.g., at average, normal, and/or high levels). In some embodiments, the affinity with which the subject multi-specific antibodies bind cells that express both ABCB1 and CD47 is greater than twice, including e.g., greater than 2.5 times, greater than 3 times, greater than 4 times, greater than 5 times, greater than 6 times, greater than 7 times, greater than 8 times, greater than 9 times, greater than 10 times, or more, as compared to the affinity with which the subject multi-specific antibodies bind cells that express either ABCB1 or CD47 (or a low level of either ABCB1 or CD47).

In some embodiments, the subject multi-specific antibodies may, when bound to a cell expressing ABCB1, prevent the functioning of the cellular ABCB protein. Accordingly, multi-specific antibodies of the present disclosure may inhibit efflux by the MDR1 protein, including e.g., where efflux is reduced by 5% or more, including e.g., 10% or more, 15% or more, 20% or more, 25% or more, 30% or more, 40% or more, 50% or more, 60% or more, 70% or more, 80% or more, or 90% or more, as compared to efflux by MDR1 in the absence of the subject multi-specific antibody.

In some embodiments, the subject multi-specific antibodies may, when bound to a cell expressing CD47, prevent the functioning of the cellular CD47 protein. Accordingly, multi-specific antibodies of the present disclosure may inhibit binding of a CD47-ligand or CD47 binding partner to CD47, including e.g., where ligand/binding partner binding is reduced by 5% or more, including e.g., 10% or more, 15% or more, 20% or more, 25% or more, 30% or more, 40% or more, 50% or more, 60% or more, 70% or more, 80% or more, or 90% or more, as compared to binding by CD47 in the absence of the subject multi-specific antibody.

Multi-specific antibodies of the present disclosure are at least bispecific for ABCB1 and CD47, where the configuration of the antibody may vary. The term “antibody” refers to a protein comprising one or more (e.g., one or two) heavy chain variable regions (VH) and/or one or more (e.g., one or two) light chain variable regions (VL), or subfragments thereof capable of binding an epitope. With regard to the herein described multi-specific antibodies, such antibodies are capable of binding at least two different epitopes present on two different target proteins. The number of different target proteins, and thus different epitopes, bound by the subject multi-specific antibodies may vary and may be two (i.e., bispecific), three (tri-specific), four, or greater.

In some embodiments, the multi-specific ABCB1XCD47 antibodies of the present disclosure may include a common light chain. As used herein, the term “common light chain” will generally refer to the use, and incorporation, of two copies of the same light chain into the multi-specific antibody. Put another way, a light chain, in the assembled multi-specific antibody, will associate with the MDR1-specific heavy chain and a second copy of the same light chain will associate with the CD47-specific heavy chain. In some antibodies, the common light chain is humanized, having one of the humanized light chain variable region sequences disclosed herein.

The VH and VL regions can be further subdivided into regions of hypervariability, termed “complementarity determining regions (CDR)”, interspersed with regions that are more conserved, termed “framework regions (FR)”. The extent of the FR and CDRs has been precisely defined (see, Kabat, et al. (1991) Sequences of Proteins of Immunological Interest, Fifth Edition, U.S. Department of Health and Human Services, NIH Publication No. 91-3242; Chothia et al. (1987) J. Mol. Biol. 196: 901-917). A VH can comprise three CDRs and four FRs arranged from N-terminus to C-terminus in the following order: FR1, CDR1, FR2, CDR2, FR3, CDR3, FR4. Similarly, a VL can comprise three CDRs and four FRs arranged from N-terminus to C-terminus in the following order: FR1, CDR1, FR2, CDR2, FR3, CDR3, FR4.

The VH or VL chain of an antibody can further include all or part of a heavy or light chain constant region, to thereby form a heavy or light immunoglobulin chain, respectively. In one embodiment, the antibody is a tetramer of two heavy and two light chains, wherein the heavy and light chains are interconnected by, for example, disulphide bonds. The heavy chain constant region is comprised of three domains, CH1, CH2 and CH3. The light chain constant region is comprised of one domain, CL. The variable regions of the heavy and light chains comprise binding regions that interact with antigen. The constant regions of the antibodies typically mediate the binding of the antibody to host tissues and factors, including various cells of the immune system and the first component of the complement system.

The bispecific humanized ABCB1 x CD47 antibodies herein include antibody molecules in a variety of formats including, without limitation, the formats illustrated in FIGS. 1, 2A and 2B.

In some embodiments, a subject antibody does not comprise a full-length immunoglobulin heavy chain and a full-length immunoglobulin light chain, and instead comprises antigen-binding fragments of one or more full-length immunoglobulin heavy chains and/or one or more antigen-binding fragments of a full-length immunoglobulin light chain. In some embodiments, the antigen-binding fragments are contained on separate polypeptide chains; in other embodiments, the antigen-binding fragments are contained within a single polypeptide chain.

The term “antigen-binding fragment” refers to one or more fragments of a full-length antibody that are capable of specifically binding to ABCB1 or CD47 as described above. Examples of binding fragments include (i) a Fab fragment (a monovalent fragment including, e.g., consisting of, the VL, VH, CL and CH1 domains; (ii) a F(ab′)₂ fragment (a bivalent fragment comprising two Fab fragments linked by a disulfide bridge at the hinge region; (iii) a Fd fragment (including, e.g., consisting of, the VH and CH1 domains); (iv) a Fv fragment (including, e.g., consisting of, the VH and VL domains of a single arm of an antibody); (v) a dAb fragment (including, e.g., consisting of, the VH domain); (vi) an isolated CDR; (vii) a single chain Fv (scFv) (including, e.g., consisting of, the VH and VL domains of a single arm of an antibody joined by a synthetic linker using recombinant means such that the VH and VL domains pair to form a monovalent molecule); (viii) diabodies (including, e.g., consisting of, two scFvs in which the VH and VL domains are joined such that they do not pair to form a monovalent molecule; the VH of each one of the scFv pairs with the VL domain of the other scFv to form a bivalent molecule).

In some embodiments, a subject antibody is a recombinant or modified antibody. The term “recombinant” or “modified” antibody as used herein is intended to include all antibodies that are prepared, expressed, created, or isolated by recombinant means, such as (i) antibodies expressed using a recombinant expression vector transfected into a host cell; (ii) antibodies isolated from a recombinant, combinatorial antibody library; (iii) antibodies isolated from an animal (e.g. a mouse) that is transgenic for human immunoglobulin genes; or (iv) antibodies prepared, expressed, created, or isolated by any other means that involves splicing of human immunoglobulin gene sequences to other DNA sequences. Such recombinant antibodies include humanized, CDR grafted, chimeric, deimmunized, and in vitro generated antibodies; and can optionally include constant regions derived from human germline immunoglobulin sequences.

Modified antibodies may include modified domains, including where any antibody domain may be modified from a naturally occurring form. In some embodiments, a modified antibody may include a modified heavy chain, including a modified Fc domain as described throughout the disclosure, but may include modifications in other domains, such as the CH2 and/or CH3 domain. In some instances, modified Fc domains may employ electrostatic steering effects, including but not limited to e.g., through the use of the procedures described in Gunasekeran et al, (2010) Journal of Biological Chemistry 285, 19637-19646; the disclosure of which is incorporated herein by reference in its entirety. In some instances, a bispecific antibody is assembled through charge pair substitutions at the CH3 domain, including but not limited to e.g., where one heavy chain is modified to contain K392D and K409D substitutions and the other heavy chain is modified to contained E356K and D399K substitutions. Charge pair substituted chains may preferentially form a heterodimer with one another. The numbering of the amino acid substitutions is per EU numbering system for HCs.

### Anti-MDR1 and Anti-PD-Li (ABCB1 X PD-Li) Bispecific Antibodies

In certain aspects, the TAA may be Programmed death-ligand 1 (PD-L1). PD-L1 is also known as cluster of differentiation 274 (CD274) or B7 homolog 1 (B7-H1). In certain aspects, the bispecific antibody molecule that binds to ABCB1 and PD-L1 comprises Fc mutations in the two heavy chain constant regions. All Fc mutations and combinations of Fc mutations hereinabove described and discussed can be present in the bispecific antibodies comprising both ABCB1—and PF-L1-binding domains, and the antibodies can be present in any formats, including the bispecific antibody formats illustrated in FIGS. 1, 2A and 2B.

Representative sequences of the anti-ABCB1 heavy and light chain sequences, including the corresponding CDRs and HIR and KbR mutations are presented in Table 3. However, such sequences are for illustration only and are not limiting. All multi-specific antibodies that bind ABCB1 and PD-L1 and comprise any of the mutations listed above in any combination are specifically included.

In one aspect a bispecific ABCBiXPD-L1 antibody molecule is humanized, includes one of the humanized common light chain sequences and one of the first heavy chain sequences described in the preceding sections and the second VH chain comprises the HCDRs 1-3 of a VH chain comprising the amino acid sequence:

(SEQ ID NO: 53)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS

The HCDRs 1-3 defined as per Kabat nomenclature are:

(SEQ ID NO: 107)
HCDR1: DSWIH
(SEQ ID NO: 108)
HCDR2: WISPYGGSTYYADSVKG
(SEQ ID NO: 109)
HCDR3: RHWPGGFDY

The second VH chain of the bispecific antibody that binds to ABCB1 and PD-L1 may have an amino acid sequence at least 80%, at least 90%, at least 95%, or a 100% identical to the amino acid sequence:

(SEQ ID NO: 53)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS

The second VH chain of the bispecific antibody that binds to ABCB1 and PD-L1 may be present in heavy chain having an amino acid sequence at least 80%, at least 90%, at least 95%, or a 100% identical to the amino acid sequence:

(SEQ ID NO: 54)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK

As described elsewhere herein, at least one, two, or all of the VL chain, the first VH chain, and the second VH may be humanized. Specific humanized heavy and light chain sequences described in the previous sections and connection with the ABCB1XCD47 bispecific antibodies, may also be present in the ABCBiXPD-L1 bispecific antibodies herein. Similarly, the ABCB1XPD-L1 bispecific antibody molecules may include any of the Fc mutations or set of Fc mutations discussed earlier, generally and specifically in connection with the ABCB1 X CD47 bispecific antibodies, and may include the anti-ABCB1 heavy chain variable region sequences specifically listed above, in Table 3 or anywhere in the disclosure.

### Anti-MDR1 and Anti-EGFR (ABCB1 X EGFR) Bispecific Antibodies

In certain aspects, the TAA may be epidermal growth factor receptor (EGFR).

In certain aspects, the bispecific antibody molecule that binds to ABCB1 and EGFR comprises Fc mutations in the two heavy chain constant regions. All Fc mutations and combinations of Fc mutations hereinabove described and discussed can be present in the bispecific antibodies comprising both ABCB1—and EGFR-binding domains, and the antibodies can be present in any formats, including the bispecific antibody formats illustrated in FIGS. 1, 2A and 2B.

Representative sequences of the anti-ABCB1 heavy and light chain sequences, including the corresponding CDRs and HIR and KbR mutations are presented in Table 3. However, such sequences are for illustration only and are not limiting. All multi-specific antibodies that bind ABCB1 and EGFR and comprise any of the mutations listed above in any combination are specifically included.

The HCDRs1-3 for the second VH chain that includes an antigen-binding site for EGFR may be derived from the VH chain of the anti-EGFR antibody necitumumab or cetuximab. The heavy chain of necitumumab has the following sequence:

(SEQ ID NO: 55)
QVQLQESGPGLVKPSQTLSLTCTVSGGSISSGDYYWSWIRQPPGKGLEWI
GYIYYSGSTDYNPSLKSRVTMSVDTSKNQFSLKVNSVTAADTAVYYCARV
SIFGVGTFDYWGQGTLVTVSSASTKGPSVLPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
K

The heavy chain of cetuximab has the following sequence:

(SEQ ID NO: 56)
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK

In a first aspect, the anti-MDR1 anti-EGFR bispecific antibody includes the VL and first VH chain as described in the preceding sections and the second VH chain may include the HCDRs from VH region of necitumumab. The HCDRs defined as per Kabat nomenclature may have the following sequences:

(SEQ ID NO: 110)
HCDR1: SGDYYWS
(SEQ ID NO: 111)
HCDR2: YIYYSGSTDYNPSLKS
(SEQ ID NO: 112)
HCDR3: VSIFGVGTFDY

In certain aspects, the second VH chain may have an amino acid sequence at least 80%, at least 90%, at least 95%, or a 100% identical to the amino acid sequence:

(SEQ ID NO: 57)
QVQLQESGPGLVKPSQTLSLTCTVSGGSISSGDYYWSWIRQPPGKGLEWI
GYIYYSGSTDYNPSLKSRVTMSVDTSKNQFSLKVNSVTAADTAVYYCARV
SIFGVGTFDYWGQGTLVTVSS

In a second aspect, the anti-ABCB1 anti-EGFR bispecific antibody includes the VL and first VH chain as described in the preceding sections and the second VH chain may include the HCDRs from VH region of cetuximab. The HCDRs defined as per Kabat nomenclature may have the following sequences:

(SEQ ID NO: 113)
HCDR1: NYGVH
(SEQ ID NO: 114)
HCDR2: VIWSGGNTDYNTPFTS
(SEQ ID NO: 115)
HCDR3: ALTYYDYEFAY

In certain aspects, the second VH chain may have an amino acid sequence at least 80%, at least 90%, at least 95%, or a 100% identical to the amino acid sequence:

(SEQ ID NO: 58)
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSA

The anti-ABCB1 anti-EGFR bispecific antibodies described above may include the same combination of VH and VL chains as the anti-ABCB1 anti-CD47 bispecific antibodies and the anti-MDR1 anti-PD-L1 bispecific antibodies described above, specifically including the Fc mutations and humanized heavy and light chain amino acid sequences disclosed therein.

As described elsewhere herein, at least one, two, or all of the VL chain, the first VH chain, and the second VH may be humanized. Specific humanized heavy and light chain sequences described in the previous sections and connection with the ABCB1XCD47 bispecific antibodies, may also be present in the ABCB1XEGFR bispecific antibodies herein. Similarly, the ABCB1XEGFR bispecific antibody molecules may include any of the Fc mutations or set of Fc mutations discussed earlier, generally and specifically in connection with the ABCB1 X CD47 bispecific antibodies and may include the anti-ABCB1 heavy chain variable region sequences specifically listed above, in Table 3 or anywhere in the disclosure.

### Additional Anti-ABCB1 Anti-TAA Bispecific Antibodies

Further bispecific antibodies disclosed herein include a VH and VL chain as described herein that specifically binds to ABCB1 and a VH chain and VL chain that binds to a TAA. The TAA may be an antigen expressed on the surface of a multi-drug resistant cancer cell and/or co-expressed with ABCB1 on the surface of a cancer cell. In certain embodiments, the VH chain that binds to a TAA may include HCDRs1-3 or the VH sequence of an anti-TAA antibody as set forth in Table 4.

TABLE 4

| TAA | Antibodies | Sequence of VH |
|---|---|---|
| CD47 | Magrolimab | QVQLVQSGAEVKKPGASVKVSCKASGYTFTNYNMHWVRQAPGQRL |
| | | EWMGTIYPGNDDTSYNQKFKDRVTITADTSASTAYMELSSLRSEDTA |
| | | VYYCARGGYRAMDYWGQGTLVTVSS (SEQ ID NO: 11) |
| HER2 | Tratuzumab | EVQLVESGGGLVQPGGSLRLSCAASGFNIKDTYIHWVRQAPGKGLEW |
| | | VARIYPTNGYTRYADSVKGRFTISADTSKNTAYLQMNSLRAEDTAVY |
| | | YCSRWGGDGFYAMDYWGQGTLVTVSS (SEQ ID NO: 59) |
| HER2 | Pertuzumab | EVQLVESGGGLVQPGGSLRLSCAASGFTFTDYTMDWVRQAPGKGLE |
| | | WVADVNPNSGGSIYNQRFKGRFTLSVDRSKNTLYLQMNSLRAEDTA |
| | | VYYCARNLGPSFYFDYWGQGTLVTVSS (SEQ ID NO: 60) |
| EGFR | Cetuximab | QVQLKQSGPGLVQPSQSLSITCTVSGFSLTNYGVHWVRQSPGKGLEW |
| | | LGVIWSGGNTDYNTPFTSRLSINKDNSKSQVFFKMNSLQSNDTAIYYC |
| | | ARALTYYDYEFAYWGQGTLVTVS (SEQ ID NO: 61) |
| EGFR | Necitumumab | QVQLQESGPGLVKPSQTLSLTCTVSGGSISSGDYYWSWIRQPPGKGLE |
| | | WIGYIYYSGSTDYNPSLKSRVTMSVDTSKNQFSLKVNSVTAADTAVY |
| | | YCARVSIFGVGTFDYWGQGTLVTVSS (SEQ ID NO: 57) |
| PD-L1 | atezolizumab | EVQLVESGGGLVQPGGSLRLSCAASGFTFSDSWIHWVRQAPGKGLE |
| | | WVAWISPYGGSTYYADSVKGRFTISADTSKNTAYLQMNSLRAEDTA |
| | | VYYCARRHWPGGFDYWGQGTLVTVSS (SEQ ID NO: 53) |
| PD-L1 | Avelumab | EVQLLESGGGLVQPGGSLRLSCAASGFTFSSYIMMWVRQAPGKGLE |
| | | WVSSIYPSGGITFYADTVKGRFTISRDNSKNTLYLQMNSLRAEDTAVY |
| | | YCARIKLGTVTTVDYWGQGTLVTVSS (SEQ ID NO: 62) |
| PD-L1 | Durvalumab | EVQLVESGGGLVQPGGSLRLSCAASGFTFSRYWMSWVRQAPGKGLE |
| | | WVANIKQDGSEKYYVDSVKGRFTISRDNAKNSLYLQMNSLRAEDTA |
| | | VYYCAREGGWFGELAFDYWGQGTLVTVSS (SEQ ID NO: 63) |
| AXL | YW327.6S2 | EVQLVESGGGLVQPGGSLRLSCAASGFSLSGSWIHWVRQAPGKGLE |
| | (GNE) | WVGWINPYRGYAYYADSVKGRFTISADTSKNTAYLQMNSLRAEDTA |
| | | VYYCAREYSGWGGSSVG YAMDYWGQGTLV (SEQ ID NO: 64) |
| AXL | HuMax-AXL- | EVQLLESGGGLVQPGGSLRLSCAASGFTFSSYAMNWVRQAPGKGLE |
| | ADC | WVSTTSGSGASTYYADSVKGRFTISRDNSKNTLYLQMNSLRAEDTAV |
| | Enapotamab | YYCAKIWIAFDIWGQGTMVTVSS (SEQ ID NO: 65) |
| | AXL-107-MMAE | |
| | (Genmab) | |
| LIV1 | LadiratuzumabSG | QVQLVQSGAEVKKPGASVKVSCKASGLTIEDYYMHWVRQAPGQGLE |
| | N-LIV1A | WMGWIDPENGDTEYGPKFQGRVTMTRDTSINTAYMELSRLRSDDTA |
| | (MerckADC) | VYYCAVHNAHYGTWFAYWGQGTLVTVSS (SEQ ID NO: 66) |
| LRRC15 | ABBV-085 | ABBV-085- huAD208.4.1 |
| | (ADC) | EVQLVQSGAEVKKPGSSVKVSCKASGFTFTDYYIHWVKQAPGQGLE |
| | | WIGLVYPYIGGTNYNQKFKGKATLTVDTSTTTAYMEMSSLRSEDTAV |
| | | YYCARGDNKYDAMDYWGQGTTVTVSS (SEQ ID NO: 67) |
| | | ABBV-085-huM25 |
| | | EVQLVQSGAEVKKPGASVKVSCKASGYKFSSYWIEWVKQAPGQGLE |
| | | WIGEILPGSDTTNYNEKFKDRATFTSDTSINTAYMELSRLRSDDTAVY |
| | | YCARDRGNYRAWFGYWGQGTLVTVSS (SEQ ID NO: 68) |
| Nectin4 | Enfortumab | EVQLVESGGGLVQPGGSLRLSCAASGFTFSSYNMNWVRQAPGKGLE |
| | (ADC) PADCEV | WVSYISSSSSTIYYADSVKGRFTISRDNAKNSLSLQMNSLRDEDTAVY |
| | Seattle Genetics | YCARAYYYGMDVWGQGTTVTVSS (SEQ ID NO: 69) |
| | (Agensys/Astella) | |
| Tim-3 | Cobolimab | EVQLLESGGGLVQPGGSLRLSCAAASGFTFSSYDMSWVRQAPGKGL |
| (lymphomas) | Anaptys | DWVSTISGGGTYTYYQDSVKGRFTISRDNSKNTLYLQMNSLRAEDTA |
| | Bio/Glaxo Smith | VYYCASMDYWGQGTTVTVSS (SEQ ID NO: 70) |
| | Kline | |
| Tim-3 | Sabatolimab | Previously called MBG453 |
| (lymphomas) | (Novartis) | QVQLVQSGAEVKKPGSSVKVSCKASGYTFTSYNMHWVRQAPGQGLE |
| | | WMGDIYPGNGDTSYNQKFKGRVTITADKSTSTVYMELSSLRSEDTAV |
| | | YYCARVGGAFPMDYWGQGTTVTVSS (SEQ ID NO: 71) |
| gpNMB | Glembatumumab | QVQLQESGPGLVKPSQTLSLTCTVSGGSISSFNYYWSWIRHHPGKGLE |
| | (Celldex) | WIGYIYYSGSTYSNPSLKSRVTISVDTSKNQFSLTLSSVTAADTAVYYC |
| | | ARGYNWNYFDYWGQGTLVTVSS (SEQ ID NO: 72) |
| cMET | Telisotuzumab | Previously called ABT-700 and hz224G11 |
| | (Abbvie) | QVQLVQSGAEVKKPGASVKVSCKASGYIFTAYTMHWVRQAPGQGLE |
| | | WMGWIKPNNGLANYAQKFQGRVTMTRDTSISTAYMELSRLRSDDTA |
| | | VYYCARSEITTEFDYWGQGTLVTVSS (SEQ ID NO: 73) |
| cMET | Amivantamab | Previously called J & J 61186372 |
| | (J & J) Bispecific | QVQLVQSGAEVKKPGASVKVSCETSGYTFTSYGISWVRQAPGHGLE |
| | | WMGWISAYNGYTNYAQKLQGRVTMTTDTSTSTAYMELRSLRSDDT |
| | | AVYYCARDLRGTNYFDYWGQGTLVTVSS (SEQ ID NO: 74) |
| cMET | LY2875358 | QVQLVQSGAEVKKPGASVKVSCKASGYTFTDYYMHWVRQAPGQGL |
| | (Lilly) | EWMGRVNPNRRGTTYNQKFEGRVTMTTDTSTSTAYMELRSLRSDDT |
| | | AVYYCARANWLDYWGQGTTVTVSS (SEQ ID NO: 75) |
| CD19 | Tafasitamab | EVQLVESGGGLVKPGGSLKLSCAASGYTFTSYVMHWVRQAPGKGLE |
| | MONJUVI | WIGYINPYNDGTKYNEKFQGRVTISSDKSISTAYMELSSLRSEDTAMY |
| | Xmab5574 | YCARGTYYYGTRVFDYWGQGTLVTVSS (SEQ ID NO: 76) |
| | (Xencor) | |
| CD19 | COLTUXIMAB | QVQLVQPGAEVVKPGASVKLSCKTSGYTFTSNWMHWVKQAPGQGL |
| | | EWIGEIDPSDSYTNYNQNFQGKAKLTVDKSTSTAYMEVSSLRSDDTA |
| | | VYYCARGSNPYYYAMDYWGQGTSVTVSS (SEQ ID NO: 77) |
| CD19 | Loncastuximab | QVQLVQPGAEVVKPGASVKLSCKTSGYTFTSNWMHWVKQAPGQGL |
| | | EWIGEIDPSDSYTNYNQNFQGKAKLTVDKSTSTAYMEVSSLRSDDTA |
| | | VYYCARGSNPYYYAMDYWGQGTSVTVSS (SEQ ID NO: 77) |
| CD19 | Inebilizumab | Previously called MEDI-551, Trade name Uplinza |
| | | EVQLVESGGGLVQPGGSLRLSCAASGFTFSSSWMNWVRQAPGKGLE |
| | | WVGRIYPGDGDTNYNVKFKGRFTISRDDSKNSLYLQMNSLKTEDTAV |
| | | YYCARSGFITTVRDFDYWGQGTLVTVSS (SEQ ID NO: 79) |
| CD19 | Blinatumomab | QVQLQQSGAELVRPGSSVKISCKASGYAFSSYWMNWVKQRPGQGLE |
| | BITE Amgen | WIGQIWPGDGDTNYNGKFKGKATLTADESSSTAYMQLSSLASEDSAV |
| | CD19xCD3 | YFCARRETTTVGRYYYAMDYWGQGTTVTVSS (SEQ ID NO: 80) |
| CD20 | Ocrelizumab | EVQLVESGGGLVQPGGSLRLSCAASGYTFTSYNMHWVRQAPGKGLE |
| | | WVGAIYPGNGDTSYNQKFKGRFTISVDKSKNTLYLQMNSLRAEDTA |
| | | VYYCARVVYYSNSYWYFDVWGQGTLVTVSS (SEQ ID NO: 81) |
| CD20 | Veltuzumab | QVQLQQSGAEVKKPGSSVKVSCKASGYTFTSYNMHWVKQAPGQGL |
| | | EWIGAIYPGMGDTSYNQKFKGKATLTADESTNTAYMELSSLRSEDTA |
| | | FYYCARSTYYGGDWYFDVWGQGTTVTVSS (SEQ ID NO: 82) |
| CD20 | Obinutuzumab | QVQLVQSGAEVKKPGSSVKVSCKASGYAFSYSWINWVRQAPGQGLE |
| | | WMGRIFPGDGDTDYNGKFKGRVTITADKSTSTAYMELSSLRSEDTAV |
| | | YYCARNVFDGYWLVYWGQGTLVTVSSASTKGPSVFPLAPSSKSTSG |
| | | GTAALGCLVKDYFPEPVTVSWNSGALTSGVHTFPAVLQSSGLYSLSS |
| | | VVTVPSS (SEQ ID NO: 119) |
| CD20 | Ofatumumab | EVQLVESGGGLVQPGRSLRLSCAASGFTFNDYAMHWVRQAPGKGLE |
| | | WVSTISWNSGSIGYADSVKGRFTISRDNAKKSLYLQMNSLRAEDTAL |
| | | YYCAKDIQYGNYYYGMDVWGQGTTVTVSS (SEQ ID NO: 84) |
| CD20 | Ibritumomab | QAYLQQSGAELVRPGASVKMSCKASGYTFTSYNMHWVKQTPRQGL |
| | | EWIGAIYPGNGDTSYNQKFKGKATLTVDKSSSTAYMQLSSLTSEDSA |
| | | VYFCARVVYYSNSYWYFDVWGTGTTVTVSA (SEQ ID NO: 85) |
| CD20 | Rituximab | QVQLQQPGAELVKPGASVKMSCKASGYTFTSYNMHWVKQTPGRGL |
| | | EWIGAIYPGNGDTSYNQKFKGKATLTADKSSSTAYMQLSSLTSEDSA |
| | | VYYCARSTYYGGDWYFNVWGAGTTVTVSA (SEQ ID NO: 86) |
| CD20 | Tositumomab | QAYLQQSGAELVRPGASVKMSCKASGYTFTSYNMHWVKQTPRQGL |
| | | EWIGAIYPGNGDTSYNQKFKGKATLTVDKSSSTAYMQLSSLTSEDSA |
| | | VYFCARVVYYSNSYWYFDVWGTGTTVTVSG (SEQ ID NO: 87) |
| CD20 | Ublituximab | QAYLQQSGAELVRPGASVKMSCKASGYTFTSYNMHWVKQTPRQGL |
| | | EWIGGIYPGNGDTSYNQKFKGKATLTVGKSSSTAYMQLSSLTSEDSA |
| | | VYFCARYDYNYAMDYWGQGTSVTVSS (SEQ ID NO: 89) |
| CD33 | Gentuzumab | EVQLVQSGAEVKKPGSSVKVSCKASGYTITDSNIHWVRQAPGQSLEW |
| | (Pfizer) | IGYIYPYNGGTDYNQKFKNRATLTVDNPTNTAYMELSSLRSEDTAFY |
| | | YCVNGNPWLAYWGQGTLVTVSS (SEQ ID NO: 90) |
| CD33 | Lintuzumab | QVQLVQSGAEVKKPGSSVKVSCKASGYTFTDYNMHWVRQAPGQGL |
| | (Seagen) | EWIGYIYPYNGGTGYNQKFKSKATITADESTNTAYMELSSLRSEDTAV |
| | | YYCARGRPAMDYWGQGTLVTVSS (SEQ ID NO: 91) |
| CD66e | Labetuzumab | QVQLVESGGGVVQPGRSLRLSCAASGFTFSSYDMHWVRQAPGKGLE |
| | (Immunomedics) | WVAVIWYDGSNKYYADSVKGRFTISRDNSKNTLYLQMNSLRAEDTA |
| | | VYYCARGSGNWGFFDYWGQGTLVTVSS (SEQ ID NO: 92) |
| B7-H4 | FPA150 | QIQLQQSGPEVVKPGASVKISCKASGYTFTDYYITWVKQKPGQGLEW |
| | (FivePrime) | IGWIYPGSGNTKYNEKFKGKATLTVDTSSSTAFMQLSSLTSEDTAVYF |
| | | CANYGNYWFAYWGQGTQVTVSA (SEQ ID NO: 93) |
| CD30 | XmAb2513 | QVQLVQSGAEVKKPGASVKVSCKVSGYTFTDYYITWVRQAPGQALE |
| | (Xencor) | WMGWIYPGSGNTKYSQKFQGRFVFSVDTSASTAYLQISSLKAEDTAV |
| | | YYCANYGNYWFAYWGQGTLVTVSS (SEQ ID NO: 94) |
| CD30 | MDX-060 | QVQLQQSAAELARPGASVKMSCKASGYTFTDLSMHWIKQRPGQGLE |
| | Iratumumab | WIGYITPSSGYTAYNQKFKGKTTLTADKSSSTAYMQLSSLTSEDSAVY |
| | 5f11 | YCARLILQRGAYWGQGTLVTVSA (SEQ ID NO: 95) |
| | (Medarex) | |
| CD44 | R05429083 | EVQLVESGGGLVQPGGSLRLSCATSGFDFSRYWMSWVRQAPGKGLV |
| | (Roche) | WIGEVNPDSTSINYTPSLKDQFTISRDNAKNTLYLQMNSLRAEDTAVY |
| | | YCTRPNYYGSRYHYYAMDYWGQGTLVTVSS (SEQ ID NO: 96) |
| CD44 | Bivatuzumab | QLQLQESGPGLLKPSETLSLTCTVSGGSISSPGYYGGWIRQPPGKGLE |
| | BIWA4 | WIGSIYKSGSTYHNPSLKSRVTISVDTSKNQFSLKLSSVTAADTAVYY |
| | | CTRPVVRYFGWFDPWGQGTLVTVSS (SEQ ID NO: 97) |
| CD70 | Cusatuzumab | EVQLVESGGGLVQPGGSLRLSCAASGFTFSVYYMNWVRQAPGKGLE |
| | (JNJ-74494550) | WVSDINNEGGTTYYADSVKGRFTISRDNSKNSLYLQMNSLRAEDTAV |
| | | YYCARDAGYSNHVPIFDSWGQGTLVTVSS (SEQ ID NO: 98) |
| CD70 | Vorsetuzumab | QVQLVQSGAEVKKPGASVKVSCKASGYTFTNYGMNWVRQAPGQGL |
| | | KWMGWINTYTGEPTYADAFKGRVTMTRDTSISTAYMELSRLRSDDT |
| | | AVYYCARDYGDYGMDYWGQGTTVTVSS (SEQ ID NO: 99) |
| CD73 | Oleclumab | EVQLLESGGGLVQPGGSLRLSCAASGFTFSSYAYSWVRQAPGKGLEW |
| | (MEDI9947) | VSAISGSGGRTYYADSVKGRFTISRDNSKNTLYLQMNSLRAEDTAVY |
| | | YCARLGYGRVDEWGRGTLVTVSS (SEQ ID NO: 100) |
| CD73 | Mupadolimab | QVQLVQSGAEVEKPGASVKVSCKASGYTFTSYWITWVRQAPGQGLE |
| | CPI-006 | WMGDIYPGSGNTNYNEKFKTRVTITADKSTSTAYMELSSLRSEDTAV |
| | (Corus) | YYCAKEGGLTTEDYALDYWGQGTLVTVSS (SEQ ID NO: 101) |
| CD79b | Polatuzumab | EVQLVESGGGLVQPGGSLRLSCAASGYTFSSYWIEWVRQAPGKGLE |
| | (GNEADC) | WIGEILPGGGDTNYNEIFKGRATFSADTSKNTAYLQMNSLRAEDTAV |
| | | YYCTRRVPIRLDYWGQGTLVTVSS (SEQ ID NO: 102) |
| CD115 | Emactuzumab | QVQLVQSGAEVKKPGASVKVSCKASGYTFTSYDISWVRQAPGQGLE |
| | (GNE) | WMGVIWTDGGTNYAQKLQGRVTMTTDTSTSTAYMELRSLRSDDTA |
| | | VYYCARDQRLYFDVWGQGTTVTVSS (SEQ ID NO: 103) |
| CD221 | AVE1642Enfortu | EVQLVESGGGLVQPGGSLRLSCAASGFTFSSYNMNWVRQAPGKGLE |
| | mab | WVSYISSSSSTIYYADSVKGRFTISRDNAKNSLSLQMNSLRDEDTAVY |
| | | YCARAYYYGMDVWGQGTTVTVSS (SEQ ID NO: 69) |
| CD221 | Robatumumab | EVQLVQSGGGLVKPGGSLRLSCAASGFTFSSFAMHWVRQAPGKGLE |
| | (Merck) | WISVIDTRGATYYADSVKGRFTISRDNAKNSLYLQMNSLRAEDTAVY |
| | | YCARLGNFYYGMDVWGQGTTVTVSS (SEQ ID NO: 105) |
| CD221 | Figitumumab | EVQLLESGGGLVQPGGSLRLSCTASGFTFSSYAMNWVRQAPGKGLE |
| | (Pfizer) | WVSAISGSGGTTFYADSVKGRFTISRDNSRTTLYLQMNSLRAEDTAV |
| | | YYCAKDLGWSDSYYYYYGMDVWGQGTTVTVSS (SEQ ID NO: 106) |

In certain embodiments, the VH chain that binds to a TAA may include HCDRs1-3 or the VH sequence of an anti-TAA antibody listed in Table 5.

TABLE 5

| TAA | Antibodies |
|---|---|
| LY6E | DLYE5953A |
| | RG7841 (ADC) |
| | RG7841 |
| Nectin4 | 9MW2821 (ADC) |
| | (Shanghai Mabwell) |
| Tim-3 | LY3321367 |
| (lymphomas) | (Lilly) |
| cMET | TR1801-ADC |
| cMET | F46 |
| ENPP3 | AGS-16C3F |
| | (ADC) |
| CD19 | DI-B4 |
| | (Merck Serono) |
| CD33 | BI836858 |
| | (BI) |
| CD44 | RG7356 |
| | (Roche) |
| CD79b | Polatuzumab |
| | (GNEADC) |
| CD73 | AK119 |

### Compositions and Formulations

The present disclosure provides compositions, including pharmaceutical compositions, comprising an antibody herein.

In general, a pharmaceutical formulation comprises an effective amount of the subject antibody in combination with a pharmaceutically acceptable excipient. An “effective amount” means a dosage sufficient to produce a desired result, e.g., reduction in a cancer of a subject, reduction in the growth rate of a cancer in a subject, amelioration of a symptom of cancer, and the like. Generally, the desired result is at least a reduction in a symptom of a cancer, reduction in the growth of a cancer, reduction in the size of a cancer, etc., as compared to a control. The effective amount of antibody present in the formulation is determined by considering the desired dose volumes and mode(s) of administration, for example.

The following formulations, excipients and methods are merely exemplary and are in no way limiting.

The pharmaceutical composition may, for example, be in a liquid form, a lyophilized form or a liquid form reconstituted from a lyophilized form, wherein the lyophilized preparation is to be reconstituted with a sterile solution prior to administration. Thus, formulations of the subject antibodies may be in a solid form, which present advantages, such as improved stability and increased shelf-life as well as simpler storage and transportation. Such solid formulations may be prepared by a variety of drying technologies, including lyophilization and spray dry manufacturing processes. Solid formulations may be reconstituted prior to use. The standard procedure for reconstituting a lyophilized composition is to add back a volume of pure water (typically equivalent to the volume removed during lyophilization), however, solutions comprising antibacterial agents may be used for the production of pharmaceutical compositions for parenteral administration; see also Chen (1992) Drug Dev Ind Pharm 18, 1311-54.

Thus, pharmaceutical compositions of the subject antibodies might be in a variety of formulations, including lyophilized powders and liquid, typically aqueous, formulations, which may be concentrated solutions that require dilution prior to administration or ready-to-use solutions suitable for subcutaneous administration. Generally, the compositions contain the antibody, an excipient to adjust tonicity or osmolality for solutions or a lyoprotectant for lyophilized powders, a buffer, and a surfactant. The ionic tonicity-adjusting excipient may, for example, be sodium chloride, and non-ionic osmolality-adjusting excipients include, for example, trehalose, sucrose, mannitol, maltose, and sorbitol. Typical lyoprotectants include trehalose and sucrose.

A tonicity agent may be included in the antibody formulation to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 nM.

A surfactant may also be added to the antibody formulation to reduce aggregation of the formulated antibody and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™) Examples of suitable polyethylene-polypropylene copolymers are those sold under the names Pluronic® F68 or Poloxamer 188™. Examples of suitable Polyoxyethylene alkyl ethers are those sold under the trademark Brij™. Exemplary concentrations of surfactant may range from about 0.001% to about 1% w/v.

A lyoprotectant may also be added in order to protect the labile active ingredient (e.g. a protein) against destabilizing conditions during lyophilization, if any. For example, known lyoprotectants include sugars (including trehalose, glucose and sucrose); polyols (including mannitol, sorbitol and glycerol); and amino acids (including alanine, glycine and glutamic acid). Typical lyoprotectants are trehalose and sucrose. Lyoprotectants can be included in an amount of about 10 mM to 500 nM.

Pharmaceutical formulations of the subject antibodies include aqueous formulations comprising the antibody in a pH-buffered solution. The buffer used in aqueous pharmaceutical formulations herein has a pH in the range from about 4.8 to about 8.0. In certain embodiments the pH is in the range from 5.5 to 7.0, from pH 5.5 to 6.5, in the range from pH 5.7 to 6.8, in the range from pH 5.8 to 6.5, in the range from pH 5.9 to 6.5, in the range from pH 6.0 to 6.5, or in the range from pH 6.2 to 6.5. In certain embodiments of the invention, the formulation has a pH of 6.0 or about 6.0. Examples of buffers that will control the pH within this range include sodium histidine (such as L-histidine) and phosphate. In certain embodiments, the buffer contains histidine in the concentration of about 15 mM to about 35 mM. In certain embodiments of the invention, the buffer contains histidine in the concentration of about 20 mM to about 30 mM, about 22 mM to about 28 mM, or about 25 mM. In one embodiment, the buffer is histidine in an amount of about 20 mM, pH 6.0. Alternatively, the formulation may contain a phosphate buffer, such as sodium phosphate in the concentration of about 20 mM to about 30 mM, about 22 mM to about 28 mM, or about 25 mM. In one embodiment, the buffer is sodium phosphate in an amount of about 25 mM, pH 6.2.

As another example, a subject parenteral formulation is a liquid or lyophilized formulation which may comprise, for example, about 1 mg/mL to about 200 mg/mL of a subject antibody; 0.04% Tween 20 w/v; 20 mM L-histidine; and 250 mM trehalose or sucrose; and has a pH of 5.5.

As another example, a subject parenteral formulation comprises a lyophilized formulation comprising: 1) 15 mg/mL of a subject antibody; 0.04% Tween 20 w/v; 20 mM L-histidine; and 250 mM sucrose; and has a pH of 5.5; or 2) 75 mg/mL of a subject antibody; 0.04% Tween 20 w/v; 20 mM L-histidine; and 250 mM sucrose; and has a pH of 5.5; or 3) 75 mg/mL of a subject antibody; 0.02% Tween 20 w/v; 20 mM L-histidine; and 250 mM Sucrose; and has a pH of 5.5; or 4) 75 mg/mL of a subject antibody; 0.04% Tween 20 w/v; 20 mM L-histidine; and 250 mM trehalose; and has a pH of 5.5; or 6) 75 mg/mL of a subject antibody; 0.02% Tween 20 w/v; 20 mM L-histidine; and 250 mM trehalose; and has a pH of 5.5.

As another example, a subject parenteral formulation is a liquid formulation comprising:1) 7.5 mg/mL of a subject antibody; 0.022% Tween 20 w/v; 120 mM L-histidine; and 250 125 mM sucrose; and has a pH of 5.5; or 2) 37.5 mg/mL of a subject antibody; 0.02% Tween 20 w/v; 10 mM L-histidine; and 125 mM sucrose; and has a pH of 5.5; or 3) 37.5 mg/mL of a subject antibody; 0.01% Tween 20 w/v; 10 mM L-histidine; and 125 mM sucrose; and has a pH of 5.5; or 4) 37.5 mg/mL of a subject antibody; 0.02% Tween 20 w/v; 10 mM L-histidine; 125 mM trehalose; and has a pH of 5.5; or 5) 37.5 mg/mL of a subject antibody; 0.01% Tween 20 w/v; 10 mM L-histidine; and 125 mM trehalose; and has a pH of 5.5; or 6) 5 mg/mL of a subject antibody; 0.02% Tween 20 w/v; 20 mM L-histidine; and 250 mM trehalose; and has a pH of 5.5; or 7) 75 mg/mL of a subject antibody; 0.02% Tween 20 w/v; 20 mM L-histidine; and 250 mM mannitol; and has a pH of 5.5; or 8) 75 mg/mL of a subject antibody; 0.02% Tween 20 w/v; 20 mM L histidine; and 140 mM sodium chloride; and has a pH of 5.5; or 9) 150 mg/mL of a subject antibody; 0.02% Tween 20 w/v; 20 mM L-histidine; and 250 mM trehalose; and has a pH of 5.5; or 10) 150 mg/mL of a subject antibody; 0.02% Tween 20 w/v; 20 mM L-histidine; and 250 mM mannitol; and has a pH of 5.5; or 11) 150 mg/mL of a subject antibody; 0.02% Tween 20 w/v; 20 mM L-histidine; and 140 mM sodium chloride; and has a pH of 5.5; or 12) 10 mg/mL of a subject antibody; 0.01% Tween 20 w/v; 20 mM L-histidine; and 40 mM sodium chloride; and has a pH of 5.5.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for a subject antibody may depend on the particular antibody employed and the effect to be achieved, and the pharmacodynamics associated with each antibody in the host. In pharmaceutical dosage forms, a subject antibody can be administered in conjunction with a pharmaceutically acceptable excipient, or they may also be used alone or in appropriate association, as well as in combination with other pharmaceutically active compounds.

In some embodiments, a subject antibody is formulated in a controlled release formulation. Sustained-release preparations may be prepared using methods well known in the art. Suitable examples of sustained-release preparations include semipermeable matrices of solid hydrophobic polymers containing the antibody in which the matrices are in the form of shaped articles, e.g. films or microcapsules. Examples of sustained-release matrices include polyesters, copolymers of L-glutamic acid and ethyl-L-glutamate, non-degradable ethylene-vinyl acetate, hydrogels, polylactides, degradable lactic acid-glycolic acid copolymers and poly-D-(−)-3-hydroxybutyric acid. Possible loss of biological activity and possible changes in immunogenicity of antibodies comprised in sustained-release preparations may be prevented by using appropriate additives, by controlling moisture content and by developing specific polymer matrix compositions.

Controlled release within the scope of this invention can be taken to mean any one of a number of extended release dosage forms. The following terms may be considered to be substantially equivalent to controlled release, for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release.

Further discussions of these terms may be found in Lesczek Krowczynski, Extended-Release Dosage Forms, 1987 (CRC Press, Inc.).

Intranasal formulations are also included within the scope herein. Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa. A subject antibody can be utilized in aerosol formulation to be administered via inhalation. A subject antibody can be formulated into pressurized acceptable propellants such as dichlorodifluoromethane, propane, nitrogen and the like.

Oral formulations for administering the subject antibodies are also included. Oral administration is challenging, due to the degradation or orally administered antibodies by proteolytic enzymes, such as pepsin, trypsin, chymotrypsin, carboxypeptidase and elastase. Various approaches used to include stability or orally administered antibodies include formulations in liposomes, coating polymers and genetic engineering of resistant forms.

### Dosages

A suitable dosage can be determined by an attending physician or other qualified medical personnel, based on various clinical factors. As is well known in the medical arts, dosages for any one patient depend upon many factors, including the patient's size, body surface area, age, the particular compound to be administered, sex of the patient, time, and route of administration, general health, and other drugs being administered concurrently. A subject antibody may be administered in amounts between 1 ng/kg body weight and 20 mg/kg body weight per dose, e.g., between 0.1 mg/kg body weight to 10 mg/kg body weight, e.g. between 0.5 mg/kg body weight to 5 mg/kg body weight; however, doses below or above this exemplary range are envisioned, especially considering the aforementioned factors. If the regimen is a continuous infusion, it can also be in the range of 1 μg to 10 mg per kilogram of body weight per minute.

In addition to body-weight-based dosing schedules, the antibodies herein can also be administered by fixed dosing, where the foxed dose and dosing schedule are determined and adjusted depending on the target disease.

Those of skill will readily appreciate that dose levels can vary as a function of the specific antibody, the severity of the symptoms and the susceptibility of the subject to side effects.

Preferred dosages for a given compound are readily determinable by those of skill in the art by a variety of means.

### Routes of Administration

In the subject methods, a subject antibody can be administered to the host using any convenient means capable of resulting in the desired therapeutic effect or diagnostic effect. Thus, a subject antibody is administered to an individual using any available method and route suitable for drug delivery, including in vivo and ex vivo methods, as well as systemic and localized routes of administration.

The main routes of administration for the antibodies herein incudes intravenous (IV), subcutaneous (SC), intramuscular (IM), and pulmonary administration. Thus, the subject antibodies may, for example, be delivered by intravenous infusion, subcutaneous injection, or by intranasal delivery, but other administration routes, such as oral delivery are also possible.

In particular, the subject antibodies may be administered by intravenous administration as a bolus or by continuous infusion over a period of time, by intramuscular, intraperitoneal, intracerobrospinal, subcutaneous, intra-articular, intrasynovial, intrathecal, oral, topical, or inhalation routes. In one embodiment, the antibodies are administered by intravenous administration. For such purposes, the formulation may be injected using a syringe or via an IV line, for example. In one embodiment, the formulation is administered by subcutaneous administration.

The subject antibodies may be administered by intravenous infusion typically in about 30 to 90 minutes, every week, biweekly or every three weeks. Alternatively, or in addition, the antibodies may be administered subcutaneously, as a fixed dose or body weight-adjusted dose over a shorter period of time, such as 2 to 20 minutes, or 2 to 10 minutes, or 2 to 5 minutes, weekly, once every two weeks or once every three weeks. The advantage of subcutaneous injections is that it allows the medical practitioner to perform it in a rather short intervention with the patient. Moreover, the patient can be trained to perform the subcutaneous injection by himself.

Such self-administration is particularly useful during maintenance dosing because no hospital care is needed (reduced medical resource utilization). Usually, injections via the subcutaneous route are limited to approximately 2 ml. For patients requiring multiple doses, several unit dose formulations can be injected at multiple sites of the body surface. The subject antibodies can be co-administered with other therapeutic agents, such as, for example, chemotherapeutic agents.

By “co-administering” is meant administering two (or more) drugs during the same administration, rather than sequential administration of the two or more drugs. In the case of intravenous administration, this will generally involve combining the two (or more) drugs into the same IV bag prior to co-administration but co-administration from different, separate formulations is also included.

The antibody and one or more additionally drugs may also be administered concurrently. A drug that is administered “concurrently” with one or more other drugs is administered during the same treatment cycle, on the same day of treatment as the one or more other drugs, and, optionally, at the same time as the one or more other drugs. For instance, for cancer therapies given every 3-weeks, the concurrently administered drugs are each administered on day-1 of a 3-week cycle.

### Nucleic Acids

The present disclosure provides nucleic acids comprising nucleotide sequences encoding a subject antibody. A nucleotide sequence encoding a subject antibody can be operably linked to one or more regulatory elements, such as a promoter and enhancer, that allow expression of the nucleotide sequence in the intended target cells (e.g., a cell that is genetically modified to synthesize and/or secrete the encoded antibody).

Suitable promoter and enhancer elements are known in the art. For expression in a bacterial cell, suitable promoters include, but are not limited to, lacl, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

A nucleotide sequence encoding a subject antibody can be present in an expression vector and/or a cloning vector. Where a subject antibody comprises two or more separate polypeptides, nucleotide sequences encoding the two polypeptides can be cloned in the same or separate vectors. Separate polypeptides may be expressed from a single nucleic acid or single vector using various strategies, such as separate promoters, one or more internal ribosomal entry sites (IRES), one or more self-cleaving sequences (e.g., 2A cleavage sequences, e.g., P2A, T2A, E2A, and F2A), combinations thereof, and the like. An expression vector can include a selectable marker, an origin of replication, and other features that provide for replication and/or maintenance of the vector.

Large numbers of suitable vectors and promoters are known to those of skill in the art; many are commercially available for generating a subject recombinant construct. The following vectors are provided by way of example. Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

As noted above, a subject nucleic acid comprises a nucleotide sequence encoding a subject multi-specific antibody. A subject nucleic acid can comprise a nucleotide sequence encoding heavy- and light-chain CDRs, including ABCB1 CDRs and CD47 CDRs. In some embodiments, a subject nucleic acid comprises a nucleotide sequence encoding heavy- and/or light-chain ABCB1 CDRs, where the CDR-encoding sequences are interspersed with FR-encoding nucleotide sequences. In some embodiments, a subject nucleic acid comprises a nucleotide sequence encoding heavy- and/or light-chain CD47 CDRs, where the CDR-encoding sequences are interspersed with FR-encoding nucleotide sequences. In some embodiments, the FR-encoding nucleotide sequences are human FR-encoding nucleotide sequences.

Nucleic acids, e.g., as described herein, may, in some instances, be introduced into a cell, e.g., by contacting the cell with the nucleic acid. Cells with introduced nucleic acids will generally be referred to herein as genetically modified cells. Various methods of nucleic acid delivery may be employed including but not limited to e.g., naked nucleic acid delivery, viral delivery, chemical transfection, biolistics, and the like.

### Cells

The present disclosure provides isolated genetically modified cells (e.g., in vitro cells, ex vivo cells, cultured cells, etc.) that are genetically modified with a subject nucleic acid. In some embodiments, a subject isolated genetically modified cell can produce a subject antibody. In some instances, a genetically modified cell can deliver an antibody, e.g., to a subject in need thereof. In some instances, a genetically modified cell may be used in the production, screening, and/or discovery of multi-specific antibodies. Genetically modified cells may also, in some instances, include cells where endogenous gene expression has been reduced, e.g., inhibited, knocked-down, etc., or abolished, e.g., knocked-out. Genetically modified cells may also, in some instances, include cells where expression of a gene has been enhanced, e.g., the expression of an endogenous gene is increased or the expression of a heterologous gene is increased.

Suitable cells include eukaryotic cells, such as a mammalian cell, an insect cell, a yeast cell; and prokaryotic cells, such as a bacterial cell. Introduction of a subject nucleic acid into the host cell can be effected, for example by calcium phosphate precipitation, DEAE dextran mediated transfection, liposome-mediated transfection, electroporation, or other known method.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In some instances, useful mammalian cells may include cells derived from a mammalian tissue or organ. In some instances, cells employed are kidney cells, including e.g., kidney cells of an established kidney cell line, such as HEK 293T cells.

Suitable yeast cells or fungi or algae cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like.

Suitable prokaryotic cells include, but are not limited to, any of a variety of laboratory strains of Escherichia coli, Lactobacillus sp., Salmonella sp., Shigella sp., and the like. See, e.g., Carrier et al. (1992) J. Immunol. 148:1176-1181; U.S. Pat. No. 6,447,784; and Sizemore et al. (1995) Science 270:299-302. Examples of Salmonella strains which can be employed in the present invention include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Bacillus subtilis, Pseudomonas pudita, Pseudomonas aeruginosa, Pseudomonas mevalonii, Rhodobacter sphaeroides, Rhodobacter capsulatus, Rhodospirillum rubrum, Rhodococcus sp., and the like. In some embodiments, the host cell is Escherichia coli.

In some instances, cells of the present disclosure may be immune cells. As used herein, the term “immune cells” generally includes white blood cells (leukocytes) which are derived from hematopoietic stem cells (HSC) produced in the bone marrow. “Immune cells” includes, e.g., lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), T-regulatory cells (Treg) and gamma-delta T cells. A “cytotoxic cell” includes CD8+ T cells, natural-killer (NK) cells, and neutrophils, which cells are capable of mediating cytotoxicity responses. In some instances, useful cells of the present disclosure may be immune cells expressing a multi-specific antibody or chimeric receptor incorporating binding components of the multi-specific antibody of the present disclosure and may be useful as immunotherapeutic agents. In some instances, useful cells expressing a multi-specific antibody of the present disclosure may include producer T cells. Nonlimiting examples of producer T cells include those described in Tsai & Davila Oncoimmunology. (2016) 5(5): e1122158; the disclosure of which is incorporated herein by reference in its entirety. Producer T cells engineered to include nucleic acid sequence encoding a multi-specific antibody of the present disclosure may, in some instances, be employed to deliver the antibody to a subject in need thereof.

Cells of the present disclosure also include cells genetically modified to change and/or amend expression of one or more of ABCB1 and a TAA, e.g., CD47 in the cell. Such modified cells are useful for various purposes including assaying the binding of multi-specific antibodies, including but not limited to those produced according to the description and methods provided herein. In some instances, ABCB1 may be knocked out or knocked down in a subject cell line. In some instances, CD47 may be knocked out or knocked down in a subject cell line. In some instances, ABCB1 may be constitutively or inducibly overexpressed in a subject cell line. In some instances, CD47 may be constitutively or inducibly overexpressed in a subject cell line. In some instances, both ABCB1 and CD47 may knocked down, knocked out, or constitutively or inducibly overexpressed in a subject cell line. Any convenient and appropriate method for knockdown, knockout and/or overexpression may be employed. Introduced nucleic acid may be stably integrated or present transiently.

In some embodiments, cells of the present disclosure include a genetically modified human cell line that expresses CD47 and includes an exogenous nucleic acid comprising a sequence encoding MDR1 for overexpression of ABCB1. In such cells CD47 expression may by endogenous or exogenously derived (i.e., introduced) and ABCB1 expression may be stable or transient. In some instances, cells lines of the present disclosure, that express CD47, may be configured to produce a genetically modified human cell expressing CD47 and stably overexpressing ABCB1.

Cells and cell lines of the present disclosure may be cultured, including e.g., through use of culture methods described herein. In some instances, a cell, into which nucleic acid have been introduced to genetically modify the cell, may be cultured to produce a cell line. Useful cells lines may include but are not limited to e.g., genetically modified cell lines, including human cell lines, expressing CD47 and stably over-expressing ABCB1.

Cells of the present disclosure, and cell lines thereof, may be employed in various methods of the disclosure, e.g., as test samples, controls, and the like. For example, in some instances cells in which ABCB1 and/or CD47 have been knocked out and/or knocked down may be employed as reference cells, e.g., to which the binding of a multi-specific antibody of the present disclosure may be compared. Other useful reference cells include but are not limited to e.g., non-cancerous cells, as well as normal cells and cells expressing normal levels of various proteins, including normal levels of ABCB1 and/or CD47.

### Methods

As summarized above, methods of the present disclosure include methods of contacting a cell with an antibody of the present disclosure, methods of treating a subject according to a method that involves administering to the subject an antibody of the present disclosure, methods of making elements described in the instant application, including e.g., multi-specific antibodies, compositions and formulations, nucleic acids, expression vectors, cells, and the like.

As summarized above, methods of the present disclosure include contacting a cancer cell with the multi-specific antibody of the present disclosure, e.g., to facilitate and/or enhance killing of the cancer cell. In some instances, killing of the cancer cell is mediated by an immune response or immune cell acting upon the cancer cell as a result of opsonization of the cancer cell by bispecific targeting when the two targets are co-expressed on the cancer cell. In some instances, killing of the cancer cell is mediated by an immune response or immune cell acting upon the cancer cell, e.g., as a result of masking or antagonizing of a CD47 epitope present on the surface of the cancer cell by the multi-specific antibody. In some instances, killing of the cancer cell is mediated by inhibition of cellular efflux of the cancer cell, e.g., as a result of ABCB1 antagonism on the cancer cell by the multi-specific antibody. In some instances, the cell contacted with the multi-specific antibody may be a multidrug resistant cancer cell. Methods that involve contacting a cancer cell with a multi-specific antibody of the present disclosure may or may not include contacting the cancer cell with an additional therapy or active agent, including e.g., a chemotherapeutic, an immunotherapy, radiation therapy, or the like.

Contacting a cancer cell with a multi-specific antibody of the present disclosure will generally enhance the killing of the cancer cell, e.g., as compared to the level of killing of the cancer cell in the absence of the multi-specific antibody. In some instances, where an additional active agent is employed, enhanced killing of the cancer cell may be seen as compared to the level of killing observed using the additional active agent alone. The amount of enhancement of cancer cell killing attributable to the multi-specific antibody will vary and may range from at least a 5% increase in cancer cell killing to at least 90% or more, including but not limited to e.g., at least 10%, at least 20%, at least 30%, at least 40%, at least 50%, at least 60%, at least 70%, at least 80%, etc. Such increases may be compared to contacting with one or more additional active agents alone.

Enhanced killing of a cancer cell may be assessed by a variety of means including but not limited to e.g., observational studies, in vitro cell-based cytotoxicity assays, flow cytometry, cell viability labeling (e.g., using one or more cell viability stains), and the like.

Treatment Methods The present disclosure provides methods of treating a cancer, the methods generally involving administering to an individual in need thereof (e.g., an individual having a cancer) an effective amount of a subject multi-specific antibody, alone (e.g., in monotherapy) or in combination (e.g., in combination therapy) with one or more additional therapeutic agents. Administration of a multi-specific antibody of the present disclosure may be performed by any convenient and appropriate route of delivery.

Aspects of the present disclosure include the bispecific antibody molecule according to the preceding section of the specification for use in a method of treating cancer in a subject, the method comprising administering the antibody to the subject. The method comprises administering the antibody in combination with at least one additional active agent, wherein the at least one additional active agent comprises a chemotherapeutic agent, an inhibitor of a multidrug resistance transporter, an immunotherapy agent, or a combination thereof. In certain aspects, the at least one additional active agent is a chemotherapeutic agent, optionally wherein the chemotherapeutic agent is a taxol, a vinca alkaloid, or an anthracycline. Some chemotherapeutic agents that are substrates for ABCB1 pump include paclitaxel, Colchicine, Verapamil, Vinblastine, Topotecan, Doxorubicin, Daunorubicin, Etoposide, and Nilotinib.

Also disclosed herein is a chemotherapy agent for use in a method of treating cancer in a subject, the method comprising administering the chemotherapy agent in combination with the antibody described herein to the subject, optionally wherein the chemotherapy agent is a taxol, a vinca alkaloid, or an anthracycline.

Accordingly, administration includes but is not limited to e.g., delivery of the antibody by injection, delivery of the antibody by infusion, delivery of a nucleic acid or expression vector encoding the multi-specific antibody, delivery of the antibody by administering to the subject a cell that expresses and secretes the multi-specific antibody, and the like. Administration of an agent, a nucleic acid encoding an agent, a cell expressing an agent, etc. may include contacting with the agent, contacting with the nucleic acid, contacting with the cell, etc.

In some embodiments, an effective amount of a subject multi-specific antibody is an amount that, when administered alone (e.g., in monotherapy) or in combination (e.g., in combination therapy) with one or more additional therapeutic agents, in one or more doses, is effective to reduce an adverse symptom of cancer by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the severity of the adverse symptom in the absence of treatment with the antibody.

In some embodiments, an effective amount of a subject multi-specific antibody is an amount that, when administered alone (e.g., in monotherapy) or in combination (e.g., in combination therapy) with one or more additional therapeutic agents, in one or more doses, is effective to improve the cancer (i.e., slow the growth of the cancer, stop the growth of the cancer, reverse the growth of the cancer, kill cancer cells (including tumor cells, or the like) in the individual being treated. For example, an effective amount of a subject antibody can reduce a cancer growth rate or reduce a cancer size in an individual by at least about 5%, at least about 10%, at least about 15%, at least about 20%, at least about 25%, at least about 30%, at least about 40%, at least about 50%, or more, compared to in the absence of treatment with the multi-specific antibody.

In some instances, a subject may be treated systemically, including with the subject multi-specific antibody, with or without one or more additional reagents. By “systemic treatment”, as used herein, is meant a treatment that is not directed solely to target a specific tumor (such as e.g., a primary tumor or a defined secondary tumor) or a specific cancer containing tissue (such as e.g., the liver in the case of liver cancer, the blood in the case of a blood cancer, etc.). Systemic treatments will generally be directed to the subject's body as a whole and may include but are not limited to e.g., systemic radiation therapy, systemic chemotherapy, systemic immunotherapy, combinations thereof and the like.

In some instances, a subject may be treated locally, including with the subject multi-specific antibody, with or without one or more additional reagents. By “local treatment”, as used herein, is meant a treatment that is specifically directed to the location of a tumor (such as e.g., a primary tumor or a defined secondary tumor) or specifically directed to a cancer containing tissue (such as e.g., the liver in the case of liver cancer, the blood in the case of a blood cancer, etc.). In some instances, local treatment may also be administered in such a way as to affect the environment surrounding a tumor, such as tissue surrounding the tumor, such as tissue immediately adjacent to the tumor. Local treatment will generally not affect or not be targeted to tissues distant from the site of cancer including the site of a tumor, such as a primary tumor. Useful local treatments that may be administered in addition to or in combination with a subject multi-specific antibody, e.g., include but are not limited to surgery, local radiation therapy, local cryotherapy, local laser therapy, local topical therapy, combinations thereof, and the like.

In some embodiments, a subject treatment method involves administering a subject multi-specific antibody and one or more additional therapeutic agents. Suitable additional therapeutic agents include, but are not limited to, chemotherapeutic agents, radiation therapy reagents, immunotherapy reagents, other antibody or multi-specific antibody agents, and the like. Additional therapies that may be administered to a subject before, during or after a subject administering a multi-specific antibody of the present disclosure will vary depending on numerous factors including e.g., the type of cancer, the subject's medical history, general state of health and/or any co-morbidities, and the like. Useful cancer therapies include but are not limited to e.g., radiation therapy, chemotherapy, immunotherapy, and the like.

Radiation therapy includes, but is not limited to, x-rays or gamma rays that are delivered from either an externally applied source such as a beam, or by implantation of small radioactive sources.

Suitable antibodies for use in cancer treatment include, but are not limited to, naked antibodies, e.g., trastuzumab (Herceptin), bevacizumab (Avastin™), cetuximab (Erbitux™) panitumumab (Vectibix™), Ipilimumab (Yervoy™), rituximab (Rituxan), alemtuzumab (Lemtrada™), Ofatumumab (Arzerra™), Oregovomab (OvaRex™), Lambrolizumab (MK-3475), pertuzumab (Perjeta™), ranibizumab (Lucentis™) etc., and conjugated antibodies, e.g., gemtuzumab ozogamicin (Mylortarg™), Brentuximab vedotin (Adcetris™), g0Y-labelled ibritumomab tiuxetan (Zevalin™), 131 I-labelled tositumoma (Bexxar™), etc. Suitable antibodies for use in cancer treatment also include, but are not limited to, antibodies raised against tumor-associated antigens. Such antigens include, but are not limited to, CD20, CD30, CD33, CD52, EpCAM, CEA, gpA33, Mucins, TAG-72, CAIX, PSMA, Folate-binding protein, Gangliosides (e.g., GD2, GD3, GM2, etc.), Le y, VEGF, VEGFR, Integrin alpha-V-beta-3, Integrin alpha-5-beta-1, EGFR, ERBB2, ERBB3, MET, IGF1R, EPHA3, TRAILR1, TRAILR2, RANKL, FAP, Tenascin, etc.

Conventional cancer therapies also include targeted therapies for cancer including but not limited to e.g., Ado-trastuzumab emtansine (Kadcyla) targeting HER2 (ERBB2/neu) (approved for use in Breast cancer); Afatinib (Gilotrif) targeting EGFR (HER1/ERBB1), HER2 (ERBB2/neu) (approved for use in Non-small cell lung cancer); Aldesleukin (Proleukin) targeting (approved for use in Renal cell carcinoma, Melanoma); Alectinib (Alecensa) targeting ALK (approved for use in Non-small cell lung cancer); Alemtuzumab (Campath) targeting CD52 (approved for use in B-cell chronic lymphocytic leukemia); Atezolizumab (Tecentriq) targeting PD-L1 (approved for use in Urothelial carcinoma, Non-small cell lung cancer); Avelumab (Bavencio) targeting PD-L1 (approved for use in Merkel cell carcinoma); Axitinib (Inlyta) targeting KIT, PDGFR3, VEGFR1/2/3 (approved for use in Renal cell carcinoma); Belimumab (Benlysta) targeting BAFF (approved for use in Lupus erythematosus); Belinostat (Beleodaq) targeting HDAC (approved for use in Peripheral T-cell lymphoma); Bevacizumab (Avastin) targeting VEGF ligand (approved for use in Cervical cancer, Colorectal cancer, Fallopian tube cancer, Glioblastoma, Non-small cell lung cancer, Ovarian cancer, Peritoneal cancer, Renal cell carcinoma); Blinatumomab (Blincyto) targeting CD19/CD3 (approved for use in Acute lymphoblastic leukemia (precursor B-cell)); Bortezomib (Velcade) targeting Proteasome (approved for use in Multiple myeloma, Mantle cell lymphoma); Bosutinib (Bosulif) targeting ABL (approved for use in Chronic myelogenous leukemia); Brentuximab vedotin (Adcetris) targeting CD30 (approved for use in Hodgkin lymphoma, Anaplastic large cell lymphoma); Brigatinib (Alunbrig) targeting ALK (approved for use in Non-small cell lung cancer (ALK+)); Cabozantinib (Cabometyx, Cometriq) targeting FLT3, KIT, MET, RET, VEGFR2 (approved for use in Medullary thyroid cancer, Renal cell carcinoma); Carfilzomib (Kyprolis) targeting Proteasome (approved for use in Multiple myeloma); Ceritinib (Zykadia) targeting ALK (approved for use in Non-small cell lung cancer); Cetuximab (Erbitux) targeting EGFR (HER1/ERBB1) (approved for use in Colorectal cancer, Squamous cell cancer of the head and neck); Cobimetinib (Cotellic) targeting MEK (approved for use in Melanoma); Crizotinib (Xalkori) targeting ALK, MET, ROS1 (approved for use in Non-small cell lung cancer); Dabrafenib (Tafinlar) targeting BRAF (approved for use in Melanoma, Non-small cell lung cancer); Daratumumab (Darzalex) targeting CD38 (approved for use in Multiple myeloma); Dasatinib (Sprycel) targeting ABL (approved for use in Chronic myelogenous leukemia, Acute lymphoblastic leukemia); Denosumab (Xgeva) targeting RANKL (approved for use in Giant cell tumor of the bone); Dinutuximab (Unituxin) targeting B4GALNT1 (GD2) (approved for use in Pediatric neuroblastoma); Durvalumab (Imfinzi) targeting PD-L1 (approved for use in Urothelial carcinoma); Elotuzumab (Empliciti) targeting SLAMF7 (CS1/CD319/CRACC) (approved for use in Multiple myeloma); Enasidenib (Idhifa) targeting IDH2 (approved for use in Acute myeloid leukemia); Erlotinib (Tarceva) targeting EGFR (HER1/ERBB1) (approved for use in Non-small cell lung cancer, Pancreatic cancer); Everolimus (Afinitor) targeting mTOR (approved for use in Pancreatic, gastrointestinal, or lung origin neuroendocrine tumor, Renal cell carcinoma, Nonresectable subependymal giant cell astrocytoma, Breast cancer); Gefitinib (Iressa) targeting EGFR (HER1/ERBB1) (approved for use in Non-small cell lung cancer); Ibritumomab tiuxetan (Zevalin) targeting CD20 (approved for use in Non-Hodgkin's lymphoma); Ibrutinib (Imbruvica) targeting BTK (approved for use in Mantle cell lymphoma, Chronic lymphocytic leukemia, Waldenstrom's macroglobulinemia); Idelalisib (Zydelig) targeting P13Kδ (approved for use in Chronic lymphocytic leukemia, Follicular B-cell non-Hodgkin lymphoma, Small lymphocytic lymphoma); Imatinib (Gleevec) targeting KIT, PDGFR, ABL (approved for use in GI stromal tumor (KIT+), Dermatofibrosarcoma protuberans, Multiple hematologic malignancies); Ipilimumab (Yervoy) targeting CTLA-4 (approved for use in Melanoma); Ixazomib (Ninlaro) targeting Proteasome (approved for use in Multiple Myeloma); Lapatinib (Tykerb) targeting HER2 (ERBB2/neu), EGFR (HER1/ERBB1) (approved for use in Breast cancer (HER2+)); Lenvatinib (Lenvima) targeting VEGFR2 (approved for use in Renal cell carcinoma, Thyroid cancer); Midostaurin (Rydapt) targeting FLT3 (approved for use in acute myeloid leukemia (FLT3+)); Necitumumab (Portrazza) targeting EGFR (HER1/ERBB1) (approved for use in Squamous non-small cell lung cancer); Neratinib (Nerlynx) targeting HER2 (ERBB2/neu) (approved for use in Breast cancer); Nilotinib (Tasigna) targeting ABL (approved for use in Chronic myelogenous leukemia); Niraparib (Zejula) targeting PARP (approved for use in Ovarian cancer, Fallopian tube cancer, Peritoneal cancer); Nivolumab (Opdivo) targeting PD-1 (approved for use in Colorectal cancer, Head and neck squamous cell carcinoma, Hodgkin lymphoma, Melanoma, Non-small cell lung cancer, Renal cell carcinoma, Urothelial carcinoma); Obinutuzumab (Gazyva) targeting CD20 (approved for use in Chronic lymphocytic leukemia, Follicular lymphoma); Ofatumumab (Arzerra, HuMax-CD20) targeting CD20 (approved for use in Chronic lymphocytic leukemia); Olaparib (Lynparza) targeting PARP (approved for use in Ovarian cancer); Olaratumab (Lartruvo) targeting PDGFRa (approved for use in Soft tissue sarcoma); Osimertinib (Tagrisso) targeting EGFR (approved for use in Non-small cell lung cancer); Palbociclib (lbrance) targeting CDK4, CDK6 (approved for use in Breast cancer); Panitumumab (Vectibix) targeting EGFR (HER1/ERBB1) (approved for use in Colorectal cancer); Panobinostat (Farydak) targeting HDAC (approved for use in Multiple myeloma); Pazopanib (Votrient) targeting VEGFR, PDGFR, KIT (approved for use in Renal cell carcinoma); Pembrolizumab (Keytruda) targeting PD-1 (approved for use in Classical Hodgkin lymphoma, Melanoma, Non-small cell lung cancer (PD-L1+), Head and neck squamous cell carcinoma, Solid tumors (MSI-H)); Pertuzumab (Perjeta) targeting HER2 (ERBB2/neu) (approved for use in Breast cancer (HER2+)); Ponatinib (Iclusig) targeting ABL, FGFR1-3, FLT3, VEGFR2 (approved for use in Chronic myelogenous leukemia, Acute lymphoblastic leukemia); Ramucirumab (Cyramza) targeting VEGFR2 (approved for use in Colorectal cancer, Gastric cancer or Gastroesophageal junction (GEJ) adenocarcinoma, Non-small cell lung cancer); Regorafenib (Stivarga) targeting KIT, PDGFR3, RAF, RET, VEGFR1/2/3 (approved for use in Colorectal cancer, Gastrointestinal stromal tumors, Hepatocellular carcinoma); Ribociclib (Kisqali) targeting CDK4, CDK6 (approved for use in Breast cancer (HR+, HER2−)); Rituximab (Rituxan, Mabthera) targeting CD20 (approved for use in Non-Hodgkin's lymphoma, Chronic lymphocytic leukemia, Rheumatoid arthritis, Granulomatosis with polyangiitis); Rituximab/hyaluronidase human (Rituxan Hycela) targeting CD20 (approved for use in Chronic lymphocytic leukemia, Diffuse large B-cell lymphoma, Follicular lymphoma); Romidepsin (Istodax) targeting HDAC (approved for use in Cutaneous T-cell lymphoma, Peripheral T-cell lymphoma); Rucaparib (Rubraca) targeting PARP (approved for use in Ovarian cancer); Ruxolitinib (Jakafi) targeting JAK1/2 (approved for use in Myelofibrosis); Siltuximab (Sylvant) targeting IL-6 (approved for use in Multicentric Castleman's disease); Sipuleucel-T (Provenge) targeting (approved for use in Prostate cancer); Sonidegib (Odomzo) targeting Smoothened (approved for use in Basal cell carcinoma); Sorafenib (Nexavar) targeting VEGFR, PDGFR, KIT, RAF (approved for use in Hepatocellular carcinoma, Renal cell carcinoma, Thyroid carcinoma); Temsirolimus (Torisel) targeting mTOR (approved for use in Renal cell carcinoma); Tositumomab (Bexxar) targeting CD20 (approved for use in Non-Hodgkin's lymphoma); Trametinib (Mekinist) targeting MEK (approved for use in Melanoma, Non-small cell lung cancer); Trastuzumab (Herceptin) targeting HER2 (ERBB2/neu) (approved for use in Breast cancer (HER2+), Gastric cancer (HER2+)); Vandetanib (Caprelsa) targeting EGFR (HER1/ERBB1), RET, VEGFR2 (approved for use in Medullary thyroid cancer); Vemurafenib (Zelboraf) targeting BRAF (approved for use in Melanoma); Venetoclax (Venclexta) targeting BCL2 (approved for use in Chronic lymphocytic leukemia); Vismodegib (Erivedge) targeting PTCH, Smoothened (approved for use in Basal cell carcinoma); Vorinostat (Zolinza) targeting HDAC (approved for use in Cutaneous T-cell lymphoma); Ziv-aflibercept (Zaltrap) targeting PIGF, VEGFA/B (approved for use in Colorectal cancer); and the like.

Biological response modifiers suitable for use in connection with the methods of the present disclosure include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) interferon-α.; (7) interferon-γ; (8) colony-stimulating factors; (9) inhibitors of angiogenesis; (10) poly ADP ribose polvmerase (PARP) inhibitors and (11) antagonists of tumor necrosis factor

Chemotherapeutic agents are non-peptidic (i.e., non-proteinaceous) compounds that reduce proliferation of cancer cells and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc.

Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, paclitaxel-xylose, or paclitaxel-albumin). Also included within the term “taxane” are a variety of known derivatives, including both hydrophilic derivatives, and hydrophobic derivatives and derivatives bound to proteins e.g. Abraxane described in U.S. Pat. No. 7,820,788. Other taxane derivatives include, but are not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Useful immunotherapies include: anti-PD-1/PD-L1 immunotherapies, and/or other immunotherapy targets, such as e.g., immune check point markers, such as CTLA-4, LAG-3 and TIM-3, that may be targeted in treatment methods. Anti-PD-1/PD-L1 immunotherapies which include but are not limited to e.g., those therapies that include administering to a subject an effective amount of one or more anti-PD-1/PD-L1 therapeutic antagonists where such antagonists include but are not limited to e.g., OPDIVO® (nivolumab), KEYTRUDA® (pembrolizumab), Tecentriq™ (atezolizumab), durvalumab (MED14736), avelumab (MSB0010718C), BMS-936559 (MDX-1105), CA-170, BMS-202, BMS-8, BMS-37, BMS-242 and the like.

CTLA-4, also known as CD152, binds to CD80 and CD86. Antibodies against CTLA-4 have been approved for treating some cancer types. The co-inhibitory effect of CTLA-4 with other immunotherapies make CTLA-4 a good candidate for use in combination with other immunotherapies to treat certain cancers. TIM-3 may also be targeted for immunotherapy for several cancer types.

LAG-3 is in clinical trials for treating cancers. Anti-LAG-3 immunotherapies include those that employ antagonist LAG-3 antibodies that can both activate T effector cells (by downregulating the LAG-3 inhibiting signal into pre-activated LAG-3+ cells) and inhibit induced (i.e. antigen-specific) Treg suppressive activity. Useful LAG-3 antagonistic antibodies include relatlimab (BMS-986016; developed by Bristol-Myers Squibb), IMP701 (developed by Immutep), TSR-033 (anti-LAG-3 mAb; developed by TESARO, Inc.), and the like.

Immunotherapies also include T cell-based immunotherapies such as e.g., adoptive cell therapy (ACT) and chimeric antigen receptor (CAR) T cell therapies. For example, a subject may be administered a population of CAR T cells engineered to target an antigen expressed by the subject's cancer. A T cell-based therapy may involve, in some instances, obtaining a cellular sample from a subject, such as a blood sample or a tumor biopsy, and culturing immune cells from the sample ex vivo, with or without genetic modification of the cultured immune cells. As an example, immune cells may be obtained from a subject, cultured ex vivo and modified with a CAR specific for an antigen expressed by the cancer to produce a population of CAR T cells. Then, the CAR T cells may be reintroduced into the subject to target the cancer. T cell-based immunotherapies may be configured in various ways, e.g., by targeting various antigens, by collecting/culturing various cell types, etc., depending on a particular cancer to be treated. In addition, T cell-based immunotherapies may be administered systemically, e.g., by intravenous injection, or locally, e.g., by infusion (e.g., intraperitoneal infusion, pleural catheter infusion, etc.), direct injection, and the like.

In some instances, a method of treatment described herein may include administering to a subject one or more inhibitors of a multidrug resistance transporter, including but not limited to e.g., a multidrug resistance transporter other than MDR1. Useful inhibitors of multidrug resistance transporters include e.g., tyrosine kinase inhibitors, natural products, microRNAs, and small molecule inhibitors. Inhibitors of multidrug resistance transporters include ABC transporter inhibitors. A summary of such MDR modulators or reverters is provided in Choi (2005), Cancer Cell Int, 5:30; the disclosure of which is incorporated by reference herein in its entirety.

Individuals suitable for treatment using a method of the present disclosure include an individual having a cancer; an individual diagnosed as having a cancer; an individual being treated for a cancer with chemotherapy, radiation therapy, antibody therapy, surgery, etc.); an individual who has been treated for a cancer (e.g., with one or more of chemotherapy, radiation therapy, antibody therapy, surgery, etc.), and who has failed to respond to the treatment; an individual who has been treated for a cancer (e.g., with one or more of chemotherapy, radiation therapy, antibody therapy, surgery, etc.), and who initially responded to the treatment but who subsequently relapsed, i.e., the cancer recurred.

The methods of the present disclosure may be employed to target and treat a variety of cancers, including e.g., primary cancer, secondary cancers, re-growing cancers, recurrent cancers, refractory cancers and the like. For example, in some instances, the methods of the present disclosure may be employed as an initial treatment of a primary cancer identified in a subject. In some instances, the methods of the present disclosure may be employed as a non-primary (e.g., secondary or later) treatment, e.g., in a subject with a cancer that is refractory to a prior treatment, in a subject with a cancer that is re-growing following a prior treatment, in a subject with a mixed response to a prior treatment (e.g., a positive response to at least one tumor in the subject and a negative or neutral response to at least a second tumor in the subject), and the like.

In some instances, the methods of the present disclosure may be employed to treat a subject with a drug resistant cancer, such as a multi-drug resistant cancer. Multidrug resistance (MDR) is the mechanism by which many cancers develop resistance to chemotherapy drugs, resulting in minimal cell death and the expansion of drug-resistant tumors. MDR cancers may involve one or more resistance mechanisms including but not limited to e.g., increased expression of efflux pumps, decreased absorption of drug, inhibition of cell death or apoptosis, modulating drug metabolism, and the like. In some instances, the methods of the present disclosure may prevent, reverse or circumvent MDR.

In some instances, methods of the present disclosure may include treating a subject having a cancer that is resistant to a first agent with an effective amount of a subject multi-specific antibody described herein in combination with a second agent that is different from the first agent. For example, in some instances, cancer of a subject may be resistant to a first chemotherapeutic and the subject may be treated by administering an effective amount of a subject multi-specific antibody as described herein in combination with a second chemotherapeutic that is different from the first. Various combinations of first and second chemotherapeutics may be employed depending on e.g., the type of cancer to be treated, the likelihood of developing resistance, etc.

Numerous cancers are known to develop drug resistance. For this and other reasons the methods of the present disclosure may find use in treating various cancers including but not limited to, e.g., Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, etc.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenstr6m, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sézary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenstrōm Macroglobulinemia, Wilms Tumor, and the like.

The methods of treating described herein may, in some instances, be performed in a subject that has previously undergone one or more conventional treatments. For example, in the case of oncology, the methods described herein may, in some instances, be performed following a conventional cancer therapy including but not limited to e.g., conventional chemotherapy, conventional radiation therapy, conventional immunotherapy, surgery, etc. In some instances, the methods described herein may be used when a subject has not responded to or is refractory to a conventional therapy. In some instances, the methods described herein may be used when a subject has responded to a conventional therapy.

In some instances, the method of the present disclosure may be employed to target, treat or clear a subject for minimal residual disease (MRD) remaining after a prior cancer therapy. Targeting, treating and/or clearance of MRD may be pursued using the instant methods whether the MRD is or has been determined to be refractory to the prior treatment or not. In some instances, a method of the present disclosure may be employed to target, treat and/or clear a subject of MRD following a determination that the MRD is refractory to a prior treatment or one or more available treatment options other than those employing the herein described multi-specific antibodies.

In some instances, the instant methods may be employed prophylactically for surveillance. For example, a subject in need thereof may be administered a treatment involving one or more of the herein described multi-specific antibodies when the subject does not have detectable disease but is at risk of developing a recurrent cancer, including e.g., a drug resistant cancer. In some instances, a prophylactic approach may be employed when a subject is at particularly high risk of developing a primary cancer that would be predicted to be drug resistant or expected to become drug resistant. In some instances, a prophylactic approach may be employed when a subject has been previously treated for a cancer and is at risk of reoccurrence or development of drug resistance.

In some instances, methods of the present disclosure may involve analyzing a cancer for expression of one or more markers or therapeutic targets. For example, in some instances, methods may involve analyzing a sample of a cancer from a subject to determine whether the cancer expresses MDR1 above a predetermined threshold, a TAA (e.g., CD47, PD-L1, or EGFR) above a predetermined threshold, or both.

In some instances, whether a subject is treated with a multi-specific antibody of the present disclosure may depend on the results of the TAA and/or MDR1 testing. For example, in some instances, if a cancer expresses the TAA at or above a predetermined threshold then the subject may be treated with a multi-specific antibody of the present disclosure, and if the cancer expresses the TAA below the predetermined threshold then the subject may not be treated with the multi-specific antibody, e.g., the subject may be treated with a conventional therapy for the relevant cancer without the subject multi-specific antibody. In some instances, if a cancer expresses MDR1 at or above a predetermined threshold then the subject may be treated with a multi-specific antibody of the present disclosure, and if the cancer expresses MDR1 below the predetermined threshold then the subject may not be treated with the multi-specific antibody, e.g., the subject may be treated with a conventional therapy for the relevant cancer without the subject multi-specific antibody. In some instances, if a cancer expresses both the TAA and MDR1 at or above predetermined thresholds then the subject may be treated with a multi-specific antibody of the present disclosure, and if the cancer expresses the TAA and MDR1 below the predetermined thresholds then the subject may not be treated with the multi-specific antibody, e.g., the subject may be treated with a conventional therapy for the relevant cancer without the subject multi-specific antibody.

Any convenient assay may be employed for analyzing MDR1 and/or TAA levels, including but not limited to e.g., flow cytometry, nucleic acid-based assays (e.g., amplification, sequencing, etc.), cell cytometry, immunohistochemistry, and the like. Any convenient biological sample may be employed, including but not limited to e.g., cancer biopsy samples. Useful predetermined thresholds for assessing expression of one or more markers and/or targets may be determined by any convenient and appropriate method, including comparison of the measured level of expression to a corresponding control. For example, in some instances, a useful predetermined threshold for the level of MDR1 and/or TAA assayed in a sample may correspond to a level of MDR1 and/or TAA as measured in a reference cell, such as a healthy/normal cell. The TAA may be CD47, PD-L1, or EGFR.

### Methods of Making

As summarized above, methods of the present disclosure also include methods or making and/or identifying multi-specific antibodies as described herein. A subject antibody can be produced by any known method, e.g., conventional synthetic methods for protein synthesis; recombinant DNA methods; etc.

Where a subject antibody is a single chain polypeptide, it can synthesized using standard chemical peptide synthesis techniques. Where a polypeptide is chemically synthesized, the synthesis may proceed via liquid-phase or solid-phase. Solid phase polypeptide synthesis (SPPS), in which the C-terminal amino acid of the sequence is attached to an insoluble support followed by sequential addition of the remaining amino acids in the sequence, is an example of a suitable method for the chemical synthesis of a subject antibody. Various forms of SPPS, such as Fmoc and Boc, are available for synthesizing a subject antibody. Techniques for solid phase synthesis are described by Barany and Merrifield, Solid-Phase Peptide Synthesis; pp. 3-284 in The Peptides: Analysis, Synthesis, Biology. Vol. 2: Special Methods in Peptide Synthesis, Part A., Merrifield, et al. J. Am. Chem. Soc., 85: 2149-2156 (1963); Stewart et al., Solid Phase Peptide Synthesis, 2nd ed. Pierce Chem. Co., Rockford, III. (1984); and Ganesan A. 2006 Mini Rev. Med Chem. 6:3-10 and Camarero J A et al. 2005 Protein Pept Lett. 12:723-8. Briefly, small insoluble, porous beads are treated with functional units on which peptide chains are built. After repeated cycling of coupling/deprotection, the free N-terminal amine of a solid-phase attached is coupled to a single N-protected amino acid unit. This unit is then deprotected, revealing a new N-terminal amine to which a further amino acid may be attached. The peptide remains immobilized on the solid-phase and undergoes a filtration process before being cleaved off.

Standard recombinant methods can be used for production of a subject antibody. For example, nucleic acids encoding light and heavy chain variable regions, optionally linked to constant regions, are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences. The expression control sequences can be eukaryotic promoter systems in vectors capable of transforming or transfecting eukaryotic host cells (e.g., COS or CHO cells). Once the vector has been incorporated into the appropriate host, the host is maintained under conditions suitable for high level expression of the nucleotide sequences, and the collection and purification of the antibodies.

Because of the degeneracy of the code, a variety of nucleic acid sequences can encode each immunoglobulin amino acid sequence. The desired nucleic acid sequences can be produced by de novo solid-phase DNA synthesis or by polymerase chain reaction (PCR) mutagenesis of an earlier prepared variant of the desired polynucleotide. Oligonucleotide-mediated mutagenesis is an example of a suitable method for preparing substitution, deletion and insertion variants of target polypeptide DNA. See Adelman et al., DNA 2:183 (1983). Briefly, the target polypeptide DNA is altered by hybridizing an oligonucleotide encoding the desired mutation to a single-stranded DNA template. After hybridization, a DNA polymerase is used to synthesize an entire second complementary strand of the template that incorporates the oligonucleotide primer, and encodes the selected alteration in the target polypeptide DNA.

Suitable expression vectors are typically replicable in the host organisms either as episomes or as an integral part of the host chromosomal DNA. Commonly, expression vectors contain selection markers (e.g., ampicillin-resistance, hygromycin-resistance, tetracycline resistance, kanamycin resistance or neomycin resistance) to permit detection of those cells transformed with the desired DNA sequences.

Escherichia coli is an example of a prokaryotic host cell that can be used for cloning a subject antibody-encoding polynucleotide. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Other microbes, such as yeast, are also useful for expression. Saccharomyces (e.g., S. cerevisiae) and Pichia are examples of suitable yeast host cells, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

In addition to microorganisms, mammalian cells (e.g., mammalian cells grown in in vitro cell culture) can also be used to express and produce the polypeptides of the present invention (e.g., polynucleotides encoding immunoglobulins or fragments thereof). See Winnacker, From Genes to Clones, VCH Publishers, N.Y., N.Y. (1987). Suitable mammalian host cells include CHO cell lines, various Cos cell lines, HeLa cells, myeloma cell lines, and transformed B-cells or hybridomas. Expression vectors for these cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Examples of suitable expression control sequences are promoters derived from immunoglobulin genes, SV40, adenovirus, bovine papilloma virus, cytomegalovirus and the like. See Co et al., J. Immunol. 148:1149 (1992).

Once synthesized (either chemically or recombinantly), the whole antibodies, their dimers, individual light and heavy chains, or other forms of a subject antibody (e.g., scFv, etc.) can be purified according to standard procedures of the art, including ammonium sulfate precipitation, affinity columns, column chromatography, high performance liquid chromatography (HPLC) purification, gel electrophoresis, and the like (see generally Scopes, Protein Purification (Springer-Verlag, N.Y., (1982)). A subject antibody can be substantially pure, e.g., at least about 80% to 85% pure, at least about 85% to 90% pure, at least about 90% to 95% pure, or 98% to 99%, or more, pure, e.g., free from contaminants such as cell debris, macromolecules other than a subject antibody, etc.

In some embodiments, methods of generating a multi-specific antibody of the present disclosure may include producing candidate antibodies and screening for activity. Such methods may generate a multi-specific antibody that specifically binds a cell expressing both MDR1 and CD47 through the use of a series of steps. Steps of such methods may include: producing a multi-specific antibody or a plurality of antibodies that each include or are expected to include a MDR1-binding domain and a CD47-binding domain; contacting a first test cell expressing MDR1 and CD47 with the multi-specific antibody or plurality of antibodies; contacting a second cell expressing either MDR1 or CD47 with the multi-specific antibody or plurality of antibodies; comparing the binding of the multi-specific antibody, or the antibodies of the plurality, to the first cell with the binding of the multi-specific antibody to the second cell to determine a binding-specificity ratio; and identifying the multi-specific antibody, or one or more of the antibodies of the plurality, as specific for the cell expressing both MDR1 and CD47 when the ratio is above a predetermined threshold. Where such a threshold for comparative binding is employed, the threshold may vary and may range from 1.5:1 or more, including but not limited to e.g., 2:1, 3:1, 4:1, 5:1, 6:1, 7:1, 8:1, 9:1, 10:1, 20:1, 50:1, 100:1, etc.

Various cells may be used in such methods, including but not limited to e.g., the cells described herein. In some instances, the binding of the antibody to both MDR1-only expressing cells and CD47-only expressing cells may be performed. For example, in some instances, the method may include, relative to the steps describe above, where the second cell expresses MDR1 and not CD47 and the method further comprises contacting a third cell expressing CD47 but not MDR1 with the multi-specific antibody.

In some instances, such methods may employ one or more controls, including but not limited to e.g., control cells, control reagents, and the like. Useful control cells include those that have a known expression or known lack of expression of one or more relevant genes or proteins. Useful control reagents may include but are not limited to e.g., control antibodies such as but not limited to e.g., monospecific antibodies to known targets. For example, in some instances, such methods of the present disclosure may further include contacting the first cell, the second cell, and/or the third cell with a control antibody selected from: a monospecific anti-MDR1 antibody and a monospecific anti-CD47 antibody. Depending on the particular method used, various other or additional controls, as appropriate, may be employed.

### Kits

Aspects of the present disclosure also include kits. The kits may include, e.g., any combination of the multi-specific antibodies, reagents, compositions, formulations, cells, nucleic acids, expression vectors, or the like, described herein. A subject kit can include one or more of: a subject multi-specific antibody, a nucleic acid encoding the same, or a cell comprising a subject multi-specific nucleic acid. Kits may be configured for various purposes, including e.g., treatment kits (e.g., where a kit may include a multi-specific antibody and e.g., one or more additional active agents, such as a chemotherapeutic), kits for producing antibodies, kits for screening antibodies, and the like.

Optional components of the kit will vary and may, e.g., include: a buffer; a protease inhibitor; etc. Where a subject kit comprises a subject nucleic acid, the nucleic acid may also have restrictions sites, multiple cloning sites, primer sites, etc. The various components of the kit may be present in separate containers or certain compatible components may be pre-combined into a single container, as desired.

In addition to above-mentioned components, a subject kit can include instructions for using the components of the kit to practice a subject method. The instructions for practicing a subject method are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. compact disc-read only memory (CD-ROM), digital versatile disk (DVD), diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In one embodiment, kits with unit doses of a subject antibody, e.g. in injectable doses, are provided. In some embodiments, in addition to the containers containing the unit doses will be an informational package insert describing the use and attendant benefits of the antibody in treating pathological condition of interest. If the antibody is administered in combination with another drug, the article of manufacture ma comprise two vials, where the first vial contains the subject antibody and the second vial contains the other drug, such as a chemotherapeutic agent or another antibody.

In one embodiment of an article of manufacture herein comprises an intravenous (IV) bag containing a stable formulation of a subject antibody suitable for administration to a cancer patient.

Optionally, the formulation in the IV bag is stable for up to 24 hours at 5° C. or 30° C. . Stability of the formulation can be evaluated by one or more assays such as, color, appearance and clarity (CAC), concentration and turbidity analysis, particulate analysis, size exclusion chromatography (SEC), ion-exchange chromatography (IEC), capillary zone electrophoresis (CZE), image capillary isoelectric focusing (iCIEF), and potency assay.

The present invention is described in further detain in the following examples which are not in any way intended to limit the scope of the invention as claimed. The attached Figures are meant to be considered as integral parts of the specification and description of the invention. All references cited are herein specifically incorporated by reference for all that is described therein. The following examples are offered to illustrate, but not to limit the claimed invention.

### EXAMPLES

The following examples are put forth to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

For easy reference, the full and short names of the various ABCB1 X CD47 antibodies disclosed and tested are listed below.

| Full name | Short name | Humanized |
|---|---|---|
| 15D3DD KT14KK MRK16 | KB1-1401 | No |
| 15D3 hmz HIR2/KT14 KBR1/MRK16v4b | KB-1418 | Yes |
| 15D3hmzG1 HIR2/MRK16v4b hmz | KB1-1418-B1SA | Yes |
| K14 hG1 KbR1/MRK 16v4b hmz SA | KB1-1418-CD47SA | Yes |
| 15D3hmz HIR2/KT14 KbR1/B1VL6/CDR3v2a | KB-1419 | Yes |
| 15D3hmzhG1 HIR2/Fc/B1 VL6/CDR3v2a hmz SA | KB1-1419-B1SA | Yes |
| FC KT14 hG1 KbR1/B1 VL6/CDR3v2a hmz SA | KB1-1419-CD47SA | Yes |
| 15D3 hmz HIR2/KT14 KbR1/B1.89v1.huL1(KV2) | KB-1420 | Yes |
| 15D3hmzhG1 HIR2/Fc/B1.89v1 huL1(KV2) hmz | KB1-1420-B1SA | Yes |
| Fc/KT14/B1.89v1 huL1(KV2) hmz LC | KB1-1420-CD47SA | Yes |
| 15D3VHhuIgG1DD/KT14KK/B1.89v1.huL1(KV2)hmz | KB1-1421 | Yes |
| 15D3DDIgG1/KT14VHKKIgG1/89v1VL in huIgG1 | KB1-1422 | No |
| 15D3hmzhG1 HIR2/Fc/B1.89v1 huL1(KV2) hmz | KB1-1423SA | Yes |
| Fc/KT14/B1.89v1 huL1(KV2) hmz LC | KB1-1424SA | Yes |

SA = single arm

### Example 1

### Generation and Sequences of Bispecific ABCB1 X TAA Antibodies (BsAbs)

### Cell Culture and Antibody Production

Standard cell culture techniques are used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

### Expression Vectors

For the generation of the antibody expression vectors, the variable regions of heavy and light chain DNA sequences were subcloned in frame with either the human IgG1 constant heavy chain or the human IgG1 kappa constant light chain pre-inserted into the respective generic recipient expression vector optimized for expression in mammalian cell lines. The genes to be expressed were cloned into the pCl-neo Mammalian Expression Vector (Promega) that uses the full-length human cytomegalovirus (CMV) immediate-early promoter for high level gene expression. The three antibody chains were cloned into three different vectors.

The N-terminal signal sequences from mouse IgG heavy chain and kappa light chain were used for the secreted expression of the heavy and light chain, respectively. The signal peptide was cleaved during expression, leaving intact N-terminus. In the Fab constructs, the C-terminus of the CH1 IgG1 constant region was fused with a 6x His tag for purification.

Monoclonal antibodies raised against Pgp were cloned as recombinant engineered antibodies into a Human IgG1/Kappa expression vector.

Variable heavy and light fragments from mouse hybridoma sequences were obtained and were cloned into the same background of leader sequence and constant region.

Production of mAb, Fab′2, Fab, and Bispecific mAb

Antibody constructs were expressed using polymer-based co-transfection of Expi293 cells (A14527, ThermoFisher) cells growing in suspension with the mammalian expression vectors following the manufacturer's recommendations.

Bispecific heteromultimer antibodies comprise a first polypeptide (chain A) and a second polypeptide (chain B) which meet at an interface in the Fc region. Each mutation combination included cotransfection of recombinant expression vectors of the corresponding chain A and the chain B. Different cotransfection ratios of recombinant expression vectors of the chain A to that of chain B were used.

About six days after transfection the cells were harvested by centrifugation. In detail, 1 ug of total encoding DNA per 1 ml of transfected culture was diluted into of Opti-MEM® medium (Life Technologies), and incubated with Expifectamine reagent (Life Technologies) in the same medium for 20 min. The mixture was then added into the Expi293® cells growing in suspension in Expi293® Expression medium (Life Technologies) at 2.5 million cells/ml at 37° C. with and overlay of 8% of C02 in air. After 6 days, the medium containing the antibody construct was harvested by centrifugation.

### DNA Sequence Determination

DNA sequences were determined by double strand sequencing.

### DNA and Protein Sequence Analysis and Sequence Data Management

The Vector NTI (ThermoFisher) software package was used for sequence mapping, analysis, annotation and illustration.

### Sequences

(SEQ ID NO: 116)
MDLEGDRNGGAKKKNFFKLNNKSEKDKKEKKPTVSVFSMFRYSNWLDKLY
MVVGTLAAIIHGAGLPLMMLVFGEMTDIFANAGNLEDLMSNITNRSDIND
TGFFMNLEEDMTRYAYYYSGIGAGVLVAAYIQVSFWCLAAGRQIHKIRKQ
FFHAIMRQEIGWFDVHDVGELNTRLTDDVSKINEGIGDKIGMFFQSMATF
FTGFIVGFTRGWKLTLVILAISPVLGLSAAVWAKILSSFTDKELLAYAKA
GAVAEEVLAAIRTVIAFGGQKKELERYNKNLEEAKRIGIKKAITANISIG
AAFLLIYASYALAFWYGTTLVLSGEYSIGQVLTVFFSVLIGAFSVGQASP
SIEAFANARGAAYEIFKIIDNKPSIDSYSKSGHKPDNIKGNLEFRNVHFS
YPSRKEVKILKGLNLKVQSGQTVALVGNSGCGKSTTVQLMQRLYDPTEGM
VSVDGQDIRTINVRFLREIIGVVSQEPVLFATTIAENIRYGRENVTMDEI
EKAVKEANAYDFIMKLPHKFDTLVGERGAQLSGGQKQRIAIARALVRNPK
ILLLDEATSALDTESEAVVQVALDKARKGRTTIVIAHRLSTVRNADVIAG
FDDGVIVEKGNHDELMKEKGIYFKLVTMQTAGNEVELENAADESKSEIDA
LEMSSNDSRSSLIRKRSTRRSVRGSQAQDRKLSTKEALDESIPPVSFWRI
MRLNLTEWPYFVVGVFCAIINGGLQPAFAIIFSKIIGVFTRIDDPETKRQ
NSNLFSLLFLALGIISFITFFLQGFTFGKAGEILTKRLRYMVFRSMLRQD
VSWFDDPKNTTGALTTRLANDAAQVKGAIGSRLAVITQNIANLGTGIIIS
FIYGWQLTLLLLAIVPIIAIAGVVEMKMLSGQALKDKKELEGSGKIATEA
IENFRTVVSLTQEQKFEHMYAQSLQVPYRNSLRKAHIFGITFSFTQAMMY
FSYAGCFRFGAYLVAHKLMSFEDVLLVFSAVVFGAMAVGQVSSFAPDYAK
AKISAAHIIMIIEKTPLIDSYSTEGLMPNTLEGNVTFGEVVFNYPTRPDI
PVLQGLSLEVKKGQTLALVGSSGCGKSTVVQLLERFYDPLAGKVLLDGKE
IKRLNVQWLRAHLGIVSQEPILFDCSIAENIAYGDNSRVVSQEEIVRAAT
EANIHAFIESLPNKYSTKVGDKGTQLSGGQKQRIAIARALVRQPHILLLD
EATSALDTESEKVVQEALDKAREGRTCIVIAHRLSTIQNADLIVVFQNGR
VKEHGTHQQLLAQKGIYFSMVSVQAGTKRQ.

Nucleic acid sequences encoding ABCB1 (Pgp/MDR1) are available: P Glycoprotein (ABCB1) (NM_000927) Human genomic DNA Homo sapiens ATP binding cassette subfamily B member 1 (ABCB1), Ref Seq Gene on chromosome 7 (NG_011513 gen)

Nucleic acid sequences encoding CD47 are available: Homo sapiens CD47 molecule (CD47), transcript variant 1, mRNA NCBI Reference Sequence: NM_001777.3

Anti CD47 5F9 antibody variable heavy chain sequence is as follows:

(SEQ ID NO: 11)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSS.

Anti-CD47 5F9 antibody variable light chain sequence is as follows:

(SEQ ID NO: 117)
DIVMTQSPLSLPVTPGEPASISCRSSQSIVYSNGNTYLGWYLQKPGQSPQ
LLIYKVSNRFSGVPDRFSGSGSGTDFTLKISRVEADVGVYYCFQGSHVPY
TFGQGTKLEIK.

Anti-ABCB1 MRK16 antibody variable heavy chain sequence is as follows:

(SEQ ID NO: 118)
EVILVESGGGLVKPGGSLKLSCAASGFTFSSYTMSWVRQTPEKRLEWVAT
ISSGGGNTYYPDSVKGRFTISRDNAKNNLYLQMSSLRSEDTALYYCARYY
RYEAWFASWGQGTLVTVSA.

The sequence of the MRK16 antibody variable light chain is as follows:

(SEQ ID NO: 27)
DVLMTQTPVSLSVSLGDQASISCRSSQSIVHSTGNTYLEWYLQKPGQSPK
LLIYKISNRFSGVPDRFSGSGSGTDFTLKISRVEAEDLGVYYCFQASHFP
RTFGGGTKLEIK.

Anti-ABCB1 antibody 15D3 sequences are available from U.S. Pat. No. 5,849,877, wherein the antibody 15D3 Heavy chain sequence is as follows: EVKVVESGGVLVRPGGSLKLSCAASGFTFSRYTMSWVRQTPEKRLEWVATISSGGGNTYYPD SVKGRFTVSRDNAMSSLYLQMSSLRSEDTALYYCARYGAGDAWFAYWGQGTLVTVSA (SEQ ID NO:47), and the antibody 15D3 Light chain sequence is as follows:

(SEQ ID NO: 48)
DVLMTQTPLSLPVSLGDQASISCRSSQSIVHSTGNTYLEWYLQKPGQSPK
LLIYKVSNRFSGVPDRFSGSGSGTDFTLKISRLEAEDLGVYYCFQGSHFP
RTFGGGTRLEIK.

The sequence of the anti-ABCB1 9F11 antibody light chain employed as described herein is as follows:

(SEQ ID NO: 49)
DVLMTQTPLSLPVSLGDQASISCRSSQSIVHRTGNTYLEWYLQKPGQSPK
LLIYKVSNRFSGVPDRFSGSGSGTDFTLKISRVEAEDLGVYYCFQGSHVP
YTFGGGTKLEIK.

The sequence of the anti-ABCB1 9F11 antibody heavy chain employed as described herein is as follows:

(SEQ ID NO: 50)
EVKLVESGGGLVKFGGSLKLSCAASGFTLSSYYMSWVRQSPEKRLELVAV
INSNGGSTYYPDTVKGRFTISRDNAKNTLYLQMSSLKSEDTALYYCARPF
YYSNSPFAYWGQGTLVTVSS.

The sequence of the anti-ABCB1 M89 antibody light chain employed as described herein is as follows:

(SEQ ID NO: 51)
EIVLTQSPATLSLSPGERATLSCRASQSVGGSYLAWYQQKPGQAPRLLIY
GASRRATGIPARFSGSGSGTDFTLTISSLQPEDFASYFCQQTNTFPLTFG
GGTKVEIK.

The sequence of the anti-ABCB1 M89 antibody heavy chain employed as described herein is as follows:

(SEQ ID NO: 52)
QVQLVQSGGGVVQPGRSLRLSCAASGFTFSSYGMHWVRQDPGKGLMWVSS
ISTDGSATKYADSVKGRFTISRDNAKNTVSLQMNSLRAEDTAVYYCVGGF
LGWWGQGTLVTVSS.

The Fc sequences, VH and VL sequences, and full-length sequences of further bispecific antibodies described in the following examples are provided below.

TABLE 3
Fc, VL and VH sequences

| | |
|---|---|
| HC chain A | |
| HC KK | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSR |
| | KELTKNQVSLTCLVKGFYPSDIAVEWESNGQPENNYKTTPPVLKSDGS |
| | FFLYSKLTVDKSRWQQGNVFSCSVMHEALHNHYTQKSLSLSPG (SEQ |
| | ID NO: 14) |
| HIR1 | ASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRV |
| | EPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQD |
| | WLNGKEYKCKVSNKALPAPIEKTISKAKGQPREPQVCTLPPSREEMTKN |
| | QVSLSCAVKGFYPSDIAVEWESNGQPENNYKTTPPVLDSDGSFFLVSKL |
| | TVDKSRWQQGNVFSCSVMHEALHNHYTQKSLSLSPGK (SEQ ID NO: 13) |
| HIR2 | ASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRV |
| | EPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQD |
| | WLNGKEYKCKVSNKALPAPIEKTISKAKGQPREPQVCTLPPSREEMTKN |
| | QVSLSCAVKGFYPSDIAVEWESNGQPENNYKTTPPVLDSDGSFVLVSKL |
| | TVDKSRWQQGNVFSCSVMHEALHNHYTQKSLSLSPGK (SEQ ID NO: 12) |
| HIR3 | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KRVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLT |
| | VLHQDWLNGKEYKCKVSNKALPAPIEKTISKAKGQPREPQVCTLPPSR |
| | EEMTKNQVSLSCAVKGFYPSDIAVEWESNGQPENNYKTTPPVLDSDG |
| | SFVLVSDLTVDKSRWQQGNVFSCSVMHEALHNHYTQKSLSLSPGK |
| | (SEQ ID NO: 15) |
| HIR4 | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KRVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLT |
| | VLHQDWLNGKEYKCKVSNKALPAPIEKTISKAKGQPREPQVCTLPPSR |
| | EEMTKNQVSLSCAVKGFYPSDIAVEWESNGQPENNYKTTPPVLDSDG |
| | SFFLVSDLTVDKSRWQQGNVFSCSVMHEALHNHYTQKSLSLSPGK |
| | (SEQ ID NO: 16) |
| HIR5 | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KRVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLT |
| | VLHQDWLNGKEYKCKVSNKALPAPIEKTISKAKGQPREPQVCTLPPSR |
| | EEMTKNQVSLSCAVKGFYPSDIAVEWESNGQPENNYKTTPPVLKSDG |
| | SFFLVSKLTVDKSRWQQGNVFSCSVMHEALHNHYTQKSLSLSPGK |
| | (SEQ ID NO: 17) |
| HIR6 | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KRVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLT |
| | VLHQDWLNGKEYKCKVSNKALPAPIEKTISKAKGQPREPQVCTLPPSR |
| | EEMTKNQVSLSCAVKGFYPSDIAVEWESNGQPENNYKTTPPVLKSDG |
| | SFVLVSKLTVDKSRWQQGNVFSCSVMHEALHNHYTQKSLSLSPGK |
| | (SEQ ID NO: 18) |
| HIR7 | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KRVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLT |
| | VLHQDWLNGKEYKCKVSNKALPAPIEKTISKAKGQPREPRVCTLPPSR |
| | EEMTKNQVSLSCAVKGFYPSDIAVEWESNGQPENNYKTTPPVLDSDG |
| | SFFLVSKLTVDKSRWQQGNVFSCSVMHEALHNHYTQKSLSLSPGK |
| | (SEQ ID NO: 19) |
| hG1-FC KK | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KRVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSR |
| | KELTKNQVSLTCLVKGFYPSDIAVEWESNGQPENNYKTTPPVLKSDGS |
| | FFLYSKLTVDKSRWQQGNVFSCSVMHEALHNHYTQKSLSLSPG (SEQ |
| | ID NO: 20) |
| HC chain B | |
| HC DD | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSR |
| | DELTKNQVSLTCLVKGFYPSDIAVEWESNGQPENNYDTTPPVLDSDG |
| | SFFLYSDLTVDKSRWQQGNVFSCSVMHEALHNHYTQKSLSLSPG |
| | (SEQ ID NO: 21) |
| KbR1 | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KRVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPCR |
| | EEMTKNQVSLWCLVKGFYPSDIAVEWESNGQPENNYKTTPPVLDSDG |
| | SFFLYSKLTVDKSRWQQGNVFSCSVMHEALHNHYTQKSLSLSPGK |
| | (SEQ ID NO: 22) |
| KbR3 | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KRVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPCR |
| | EEMTKNQVSLWCLVKGFYPSDIAVEWESNGQPENNYKTTPPVLKSDG |
| | SFFLYSKLTVDKSRWQQGNVFSCSVMHEALHNHYTQKSLSLSPGK |
| | (SEQ ID NO: 23) |
| KbR5 | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KRVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPCR |
| | EEMTKNQVSLWCLVKGFYPSDIAVEWESNGQPENNYKTTPPVLDSDG |
| | SFFLYSDLTVDKSRWQQGNVFSCSVMHEALHNHYTQKSLSLSPGK |
| | (SEQ ID NO: 24) |
| KbR7 | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KRVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPCR |
| | EEMTENQVSLWCLVKGFYPSDIAVEWESNGQPENNYKTTPPVLDSDG |
| | SFFLYSKLTVDKSRWQQGNVFSCSVMHEALHNHYTQKSLSLSPGK |
| | (SEQ ID NO: 25) |
| hG1-FC DD | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KRVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSR |
| | DELTKNQVSLTCLVKGFYPSDIAVEWESNGQPENNYDTTPPVLDSDG |
| | SFFLYSDLTVDKSRWQQGNVFSCSVMHEALHNHYTQKSLSLSPG |
| | (SEQ ID NO: 26) |
| VL | |
| MRK16 not hmz | DVLMTQTPVSLSVSLGDQASISCRSSQSIVHSTGNTYLEWYLQKPGQ |
| | SPKLLIYKISNRFSGVPDRFSGSGSGTDFTLKISRVEAEDLGVYYCFQA |
| | SHFPRTFGGGTKLEIK (SEQ ID NO: 27) |
| MRK16 hmz | DIVMTQTPLSSPVTLGQPASISCRSSQSIVHSTGNTYLEWYQQRPGQP |
| | PRLLIYKISNRFSGVPDRFSGSGAGTDFTLKISRVEAEDVGVYYCFQAS |
| | HFPRTFGGGTKLEIKR (SEQ ID NO: 1) |
| B1VL6/CDR3v2a | DIVMTQSPLSLPVTPGEPASISCRSSQSLVHSNGNTYLEWYLQKPGQS |
| hmz | PQLLIYKVSNRFSGVPDRFSGSGSGTDFTLKISRVEAEDVGVYYCFQG |
| | SHFPRTFGQGTKLEIK (SEQ ID NO: 2) |
| MRK16v4a1 hmz | DIVMTQTPLSSPVTLGQPASISCRSSQSIVHSTGNTYLEWYQQKPGQP |
| MRK16v4b hmz | PRLLIYKISNRFSGVPDRFSGSGSGTDFTLKISRVEAEDVGVYYCFQAS |
| (same sequence) | HFPRTFGQGTKLEIK (SEQ ID NO: 3) |
| MRK16v4a12 hmz | DIVMTQTPLSSPVTLGQPASISCRSSQSIVHSTGNTYLEWYQQKPGQP |
| | PRLLIYKVSNRFSGVPDRFSGSGSGTDFTLKISRVEAEDVGVYYCFQA |
| | SHFPRTFGQGTKLEIK (SEQ ID NO: 4) |
| | DIVMTQTPLSSPVTLGQPASISCRSSQSIVHSTGNTYLEWYQQKPGQP |
| | PRLLIYKISNRFSGVPDRFSGSGSGTDFTLKISRVEAEDVGVYYCFQAS |
| | HFPRTFGQGTKLEIK |
| B1.89v1.huL1 | DIVMTQSPLSLPVTPGEPASISCRSSQTIVHSNGNTYLEWYLQKPGQS |
| (KV2) hmz | PQLLIYKVSKRFSGVPDRFSGSGSGTDFTLKISRVEAEDVGVYYCFQA |
| | SHFPRTFGQGTKLEIK (SEQ ID NO: 6) |
| VH | |
| 15D3 not hmz | EVKVVESGGVLVRPGGSLKLSCAASGFTFSRYTMSWVRQTPEKRLE |
| | WVATISSGGGNTYYPDSVKGRFTVSRDNAMSSLYLQMSSLRSEDTAL |
| | YYCARYGAGDAWFAYWGQGTLVTVSS (SEQ ID NO: 28) |
| 15D3 hmz | EVQLVESGGVVVQPGGSLRLSCAASGFTFSRYTMSWVRQAPGKGLE |
| | WVATISSGGGNTYYPDSVKGRFTVSRDNSKNSLYLQMNSLRTEDTAL |
| | YYCARYGAGDAWFAYWGQGTLVTVSS (SEQ ID NO: 9) |
| KT14 (aCD47) | QVQLVQSGAEVKKPGASVKVSCKASGYTFTNYNMHWVRQAPGQRLE |
| | WMGTIYPGNDDTSYNQKFKDRVTITADTSASTAYMELSSLRSEDTAVY |
| | YCARGGYRAMDYWGQGTLVTVSS (SEQ ID NO: 11) |
| 15D3gmz16-1 | EVQLVESGGVVVQPGGSLRLSCAASGFTFSRYTMSWVRQAPGKGLE |
| hG1 | WVATISSGGGNTYYPDSVKGRFTVSRDNSKNSLYLQMNSLRTEDTAL |
| | YYCARYYRYDAWFAYWGQGTLVTVSS (SEQ ID NO: 10) |
| VH and VL | |
| 15D3 DD hulgG1 | EVKVVESGGVLVRPGGSLKLSCAASGFTFSRYTMSWVRQTPEKRLE |
| HC | WVATISSGGGNTYYPDSVKGRFTVSRDNAMSSLYLQMSSLRSEDTAL |
| | YYCARYGAGDAWFAYWGQGTLVTVSSASTKGPSVFPLAPSSKSTSG |
| | GTAALGCLVKDYFPEPVTVSWNSGALTSGVHTFPAVLQSSGLYSLSS |
| | VVTVPSSSLGTQTYICNVNHKPSNTKVDKKVEPKSCDKTHTCPPCPAP |
| | ELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKA |
| | LPAPIEKTISKAKGQPREPQVYTLPPSRDELTKNQVSLTCLVKGFYPSD |
| | IAVEWESNGQPENNYDTTPPVLDSDGSFFLYSDLTVDKSRWQQGNVF |
| | SCSVMHEALHNHYTQKSLSLSPG (SEQ ID NO: 30) |
| 15D3 hmz DD | EVQLVESGGVVVQPGGSLRLSCAASGFTFSRYTMSWVRQAPGKGLE |
| hulgG1 HC | WVATISSGGGNTYYPDSVKGRFTVSRDNSKNSLYLQMNSLRTEDTAL |
| | YYCARYGAGDAWFAYWGQGTLVTVSSASTKGPSVFPLAPSSKSTSG |
| | GTAALGCLVKDYFPEPVTVSWNSGALTSGVHTFPAVLQSSGLYSLSS |
| | VVTVPSSSLGTQTYICNVNHKPSNTKVDKKVEPKSCDKTHTCPPCPAP |
| | ELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKA |
| | LPAPIEKTISKAKGQPREPQVYTLPPSRDELTKNQVSLTCLVKGFYPSD |
| | IAVEWESNGQPENNYDTTPPVLDSDGSFFLYSDLTVDKSRWQQGNVF |
| | SCSVMHEALHNHYTQKSLSLSPG (SEQ ID NO: 31) ABCB1KO |
| KT14 KK hulgG1 | QVQLVQSGAEVKKPGASVKVSCKASGYTFTNYNMHWVRQAPGQRLE |
| HC | WMGTIYPGNDDTSYNQKFKDRVTITADTSASTAYMELSSLRSEDTAVY |
| | YCARGGYRAMDYWGQGTLVTVSSASTKGPSVFPLAPSSKSTSGGTA |
| | ALGCLVKDYFPEPVTVSWNSGALTSGVHTFPAVLQSSGLYSLSSVVTV |
| | PSSSLGTQTYICNVNHKPSNTKVDKKVEPKSCDKTHTCPPCPAPELLG |
| | GPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPA |
| | PIEKTISKAKGQPREPQVYTLPPSRKELTKNQVSLTCLVKGFYPSDIAV |
| | EWESNGQPENNYKTTPPVLKSDGSFFLYSKLTVDKSRWQQGNVFSC |
| | SVMHEALHNHYTQKSLSLSPG (SEQ ID NO: 32) |
| 15D3 hmz HIR2 | EVQLVESGGVVVQPGGSLRLSCAASGFTFSRYTMSWVRQAPGKGLE |
| hulgG1 HC | WVATISSGGGNTYYPDSVKGRFTVSRDNSKNSLYLQMNSLRTEDTAL |
| | YYCARYGAGDAWFAYWGQGTLVTVSSASTKGPSVFPLAPSSKSTSG |
| | GTAALGCLVKDYFPEPVTVSWNSGALTSGVHTFPAVLQSSGLYSLSS |
| | VVTVPSSSLGTQTYICNVNHKPSNTKVDKRVEPKSCDKTHTCPPCPAP |
| | ELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKA |
| | LPAPIEKTISKAKGQPREPQVCTLPPSREEMTKNQVSLSCAVKGFYPS |
| | DIAVEWESNGQPENNYKTTPPVLDSDGSFVLVSKLTVDKSRWQQGNV |
| | FSCSVMHEALHNHYTQKSLSLSPG (SEQ ID NO: 33) |
| 15D3 hmz HIR4 | EVQLVESGGVVVQPGGSLRLSCAASGFTFSRYTMSWVRQAPGKGLE |
| hulgG1 HC | WVATISSGGGNTYYPDSVKGRFTVSRDNSKNSLYLQMNSLRTEDTAL |
| | YYCARYGAGDAWFAYWGQGTLVTVSSASTKGPSVFPLAPSSKSTSG |
| | GTAALGCLVKDYFPEPVTVSWNSGALTSGVHTFPAVLQSSGLYSLSS |
| | VVTVPSSSLGTQTYICNVNHKPSNTKVDKRVEPKSCDKTHTCPPCPAP |
| | ELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKA |
| | LPAPIEKTISKAKGQPREPQVCTLPPSREEMTKNQVSLSCAVKGFYPS |
| | DIAVEWESNGQPENNYKTTPPVLDSDGSFFLVSDLTVDKSRWQQGNV |
| | FSCSVMHEALHNHYTQKSLSLSPG (SEQ ID NO: 34) |
| KT14 KbR1 | QVQLVQSGAEVKKPGASVKVSCKASGYTFTNYNMHWVRQAPGQRLE |
| hulgG1 HC | WMGTIYPGNDDTSYNQKFKDRVTITADTSASTAYMELSSLRSEDTAVY |
| | YCARGGYRAMDYWGQGTLVTVSSASTKGPSVFPLAPSSKSTSGGTA |
| | ALGCLVKDYFPEPVTVSWNSGALTSGVHTFPAVLQSSGLYSLSSVVTV |
| | PSSSLGTQTYICNVNHKPSNTKVDKRVEPKSCDKTHTCPPCPAPELLG |
| | GPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPA |
| | PIEKTISKAKGQPREPQVYTLPPCREEMTKNQVSLWCLVKGFYPSDIA |
| | VEWESNGQPENNYKTTPPVLDSDGSFFLYSKLTVDKSRWQQGNVFS |
| | CSVMHEALHNHYTQKSLSLSPG (SEQ ID NO: 120) |
| KT14 KbR3 | QVQLVQSGAEVKKPGASVKVSCKASGYTFTNYNMHWVRQAPGQRLE |
| hulgG1 HC | WMGTIYPGNDDTSYNQKFKDRVTITADTSASTAYMELSSLRSEDTAVY |
| | YCARGGYRAMDYWGQGTLVTVSSASTKGPSVFPLAPSSKSTSGGTA |
| | ALGCLVKDYFPEPVTVSWNSGALTSGVHTFPAVLQSSGLYSLSSVVTV |
| | PSSSLGTQTYICNVNHKPSNTKVDKRVEPKSCDKTHTCPPCPAPELLG |
| | GPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPA |
| | PIEKTISKAKGQPREPQVYTLPPCREEMTKNQVSLWCLVKGFYPSDIA |
| | VEWESNGQPENNYKTTPPVLKSDGSFFLYSKLTVDKSRWQQGNVFS |
| | CSVMHEALHNHYTQKSLSLSPG (SEQ ID NO: 121) |
| B1.89v1.huL1 | DIVMTQSPLSLPVTPGEPASISCRSSQTIVHSNGNTYLEWYLQKPGQS |
| (KV2) LC | PQLLIYKVSKRFSGVPDRFSGSGSGTDFTLKISRVEAEDVGVYYCFQA |
| | SHFPRTFGQGTKLEIKRTVAAPSVFIFPPSDEQLKSGTASVVCLLNNFY |
| | PREAKVQWKVDNALQSGNSQESVTEQDSKDSTYSLSSTLTLSKADYE |
| | KHKVYACEVTHQGLSSPVTKSFNRGEC (SEQ ID NO: 37) |
| MRK16 LC | DVLMTQTPVSLSVSLGDQASISCRSSQSIVHSTGNTYLEWYLQKPGQS |
| | PKLLIYKISNRFSGVPDRFSGSGSGTDFTLKISRVEAEDLGVYYCFQAS |
| | HFPRTFGGGTKLEIKRTVAAPSVFIFPPSDEQLKSGTASVVCLLNNFYP |
| | REAKVQWKVDNALQSGNSQESVTEQDSKDSTYSLSSTLTLSKADYEK |
| | HKVYACEVTHQGLSSPVTKSFNRGEC (SEQ ID NO: 38) |
| B1VL6/CDR3v2a | DIVMTQSPLSLPVTPGEPASISCRSSQSLVHSNGNTYLEWYLQKPGQS |
| LC | PQLLIYKVSNRFSGVPDRFSGSGSGTDFTLKISRVEAEDVGVYYCFQG |
| | SHFPRTFGQGTKLEIKRTVAAPSVFIFPPSDEQLKSGTASVVCLLNNFY |
| | PREAKVQWKVDNALQSGNSQESVTEQDSKDSTYSLSSTLTLSKADYE |
| | KHKVYACEVTHQGLSSPVTKSFNRGEC (SEQ ID NO: 39) |

### Example 2

### Purification and Analytical Tests

### Purification

To purify antibody formats containing the Fc region, 10 μl of MabSelect™ SuRe™ (GE Healthcare) per 1 ml of supernatant were added to the harvested medium and kept stirring at 4° C. overnight. The next day, the protein A resin was applied in a 24 well filter plate using a vacuum manifold unit (Pall Lifesciences, USA). The resin was washed with PBS and the antibody eluted in 50 mM phosphate pH 3 and neutralized with 1 Ox PBS pH 13.

Fabs were purified following the same procedure using Ni Sepharose 6 Fast Flow histidine-tagged protein purification resin (GE Healthcare). The beads were washed with PBS followed by washes with 25 mM phosphate buffer pH 7.4, 150 mM NaCl supplemented with 20 mM of imidazole. The complex was eluted with 2 volumes of 25 mM phosphate buffer pH 7.4, 150 mM NaCl supplemented with 500 mM of imidazole. Finally, purified Fab was buffer-exchanged into PBS.

Analytical Test for mAbs (GXII Reduced and Non-Reduced)

Purity and monomer content of the final protein preparation was determined by high-throughput analysis on the Caliper's LabChip GXII using Protein Express LabChip Kit (Perkin-Elmer) as described by the manufacturer. The chip was automatically primed on the instrument with polymer solution containing 0.2% SDS and fluorescent staining dye. The destain channels were filled with polymer solution free of SDS and dye. Briefly, proteins in reducing and not reducing conditions were preparedptide se by mixing a small volume (2−5 pL) of sample with the caliper sample buffer with or without DDT. The samples were denatured at 75° C. for 5 minutes, centrifuged at 2000g for 3 minutes, and then run. Electropherograms were generated by LabChip GXII Touch software (Perkin Elmer).

Analytical Test for mAbs (HPLC)

Purity and monomer content of the final protein preparation was determined by high-throughput analysis on HPLC. Size exclusion chromatography (SEC) was performed using an Advancebio SEC 300A 4.6x300 mm, 2.7 um (p/n PL1580-5301) (Agilent Technologies) on an Infinity 1260 Agilent HPLC system. Injections were made under isocratic elution conditions using a mobile phase of PBS, 400 mM sodium cloride, pH 7.4, and detected with absorbance at 280 nm. Quantification is based on the relative area of detected peaks.

### CIEX HPLC

Antibody isoforms were monitored by a cation exchange (CIEX) HPLC method based on a salt gradient gradient method. In short, measurements were performed on Infinity 1260 Agilent HPLC system on a ProPac™ WCX-10G Column (10 μm, 4×260 mm) (Thermo Scientific).

Samples were diluted with 90ul 50 mM MES, 50 mM NaCl, pH 5.5 and the injection volume was 110 μl. The flow rate of the method was set to 1 ml/min. The column was equilibrated for 1 CV at 100% 50 mM MES, 50 mM NaCl, pH 5.5 and the following gradient was performed using 50 mM MES, 500 mM NaCl, pH 5.5 as solvent B.

TABLE 4

| Time | | | Flow | Max Pressure |
|---|---|---|---|---|
| (min) | A (%) | B (%) | (ml/min) | (bar) |
| 0 | 90 | 10 | 1 | 200 |
| 15 | 40 | 60 | | |
| 17 | 40 | 60 | | |
| 17.1 | 0 | 100 | | |
| 22 | 0 | 100 | | |
| 22.1 | 90 | 10 | | |
| 27 | 90 | 10 | | |

The outlet was monitored at 280 nm. The most abundant isoform is defined as main charge variant (MCV). Isoforms eluting earlier than the MCV are called acidic variants, later eluting ones are called basic variants. Injection volume and dilutions were kept constant for all samples.

### Reverse Phase HPLC

RP-HPLC was performed on an Infinity 1260 Agilent HPLC system using PLRP-S 1000A 5UM 2.1x50MM (Agilent) column. The flow rates were 0.35 mL/min. Mobile phase A was water containing 0.1% TFA (Solvent A), and solvent B was 0.1% TFA in acetonitrile. The column was initially equilibrated with 95% mobile phase A and the following gradient was performed. The absorbance was monitored at 214 and 280 nm.

TABLE 5

| Time | | | Flow | Max Pressure |
|---|---|---|---|---|
| (min) | B (%) | D (%) | (ml/min) | (bar) |
| 0 | 95 | 5 | 0.35 | 130 |
| 25 | 0 | 100 | | |
| 25.01 | 95 | 5 | | |
| 28 | 95 | 5 | | |

For non-reduced condition, 50ul samples was filtered with 0.2 um centrifugal filers at 10,000 rpm for 1 min and transferred to the analysis vials.

For the reduced condition 2ul 1 μM DTT were added into 38ul samples (50 mM DTT) and incubated at 40° C. for 5 mins.

### Liquid Chromatography-Mass Spectrometry (LC-MS).

LC-MS of Bispecific Mabs was outsourced to Novatia, LLC (Newtown, PA)

### Results

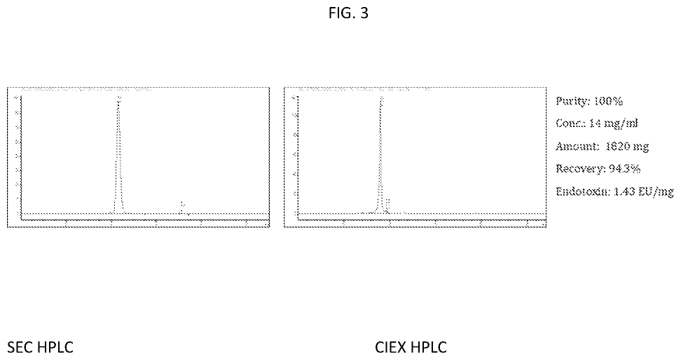

FIG. 3 demonstrates the high purity of the ABCB1XCD47 KBisP1.1 (15D3 HC KK/KT14 HC DD/MRK16 LC) bispecific antibody by SEC-HPLC and CIEX-HPLC after Protein A purification and Capto-MMC polishing. The antibody was purified to 100% purity with a recovery rate of 94.3%.

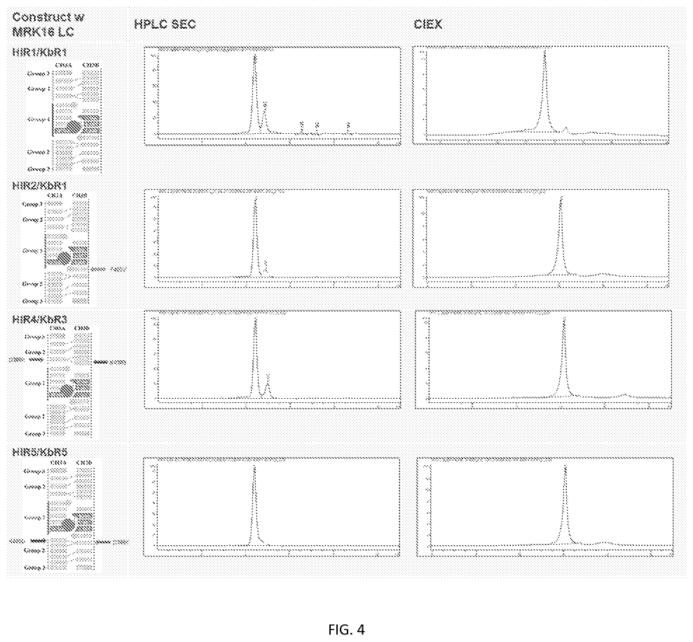

FIG. 4 demonstrates the high purity of the ABCB1xCD47 bispecific antibodies HIRX 15D3/KbRY KT14/MRK16 LC by SEC-HPLC and CIEX-HPLC after protein A purification. HIRX/KbRY=HIR1/Kbr1, HIR2/KbR1, HIR4/KbR3, or HIR5/KbR5 as shown. As evidenced by the spectra, a high degree of purity was obtained for all antibodies tested.

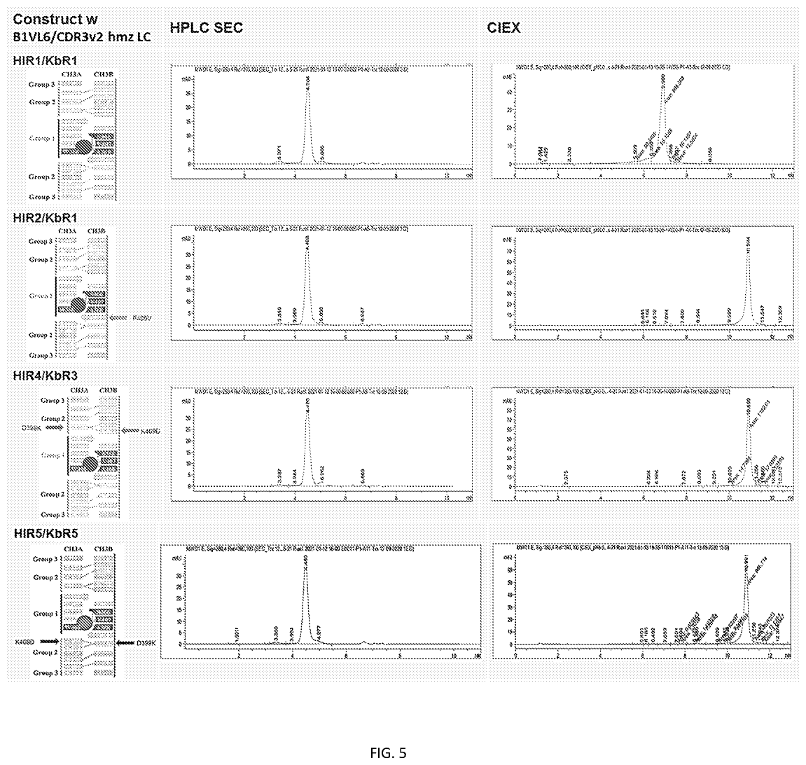

FIG. 5 demonstrates the high purity of the ABCB1xCD47 bispecific antibodies HIRX 15D3/KbRY KT14/B1VL6/CDR3v2 hmz LC by SEC-HPLC and CIEX-HPLC after Protein A purification. HIRX/KbRY=HIR1/KbR1, HIR2/KbR1, HIR4/KbR3, or HIR5/KbR5. As evidenced by the spectra, a high degree of purity was obtained for all antibodies tested.

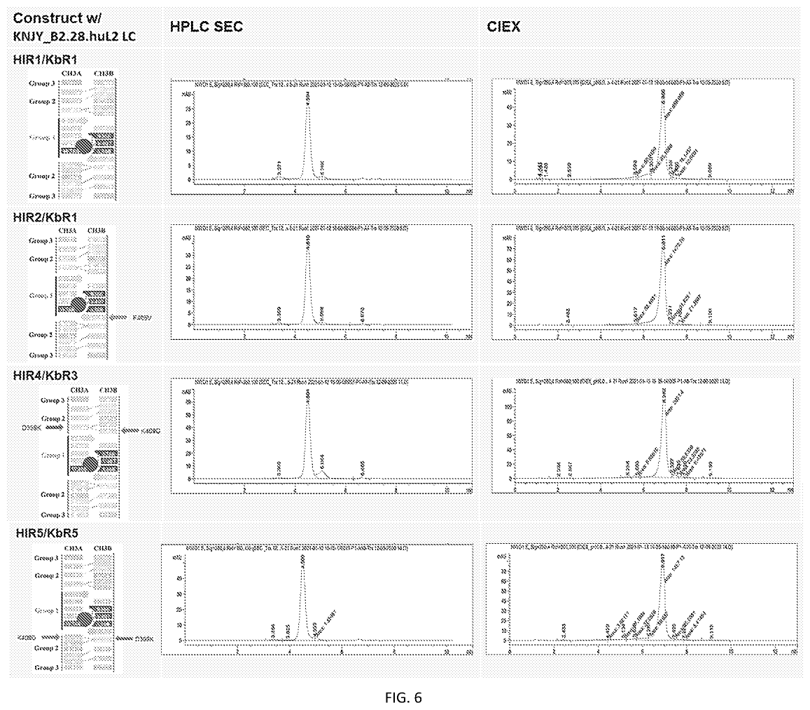

FIG. 6 demonstrates the high purity of the ABCB1XCD47 bispecific antibodies HIRX 15D3/KbR KT14/B1.28.huL2 LC by SEC/HPLC and CIEX-HPLC after protein A purification. HIRX/KbRY=HIR1/KbR1, HIR2/KbR2, HIR4/KbR3 or HIR5/KbR5. As evidenced by the spectra, a high degree of purity was obtained for all antibodies tested.

The new bispecific antibodies generated pairing the HIRX/KbRY heavy chains comprising a modified Fc region with different light chains are well expressed and can be purified to high degree of purity. The production of bispecific antibodies based on native IgG format from the use of a common light chain prevents mispairing of heavy and light chains. In particular, the achievable degree of purity of the bispecific antibodies produced with the newly engineering heavy chains HIRX/KbRY is comparable or superior to that of the KKDD bispecifics where the heavy chains are driven together by electrostatic forces and, to that of the classic knobs-into-holes approach.

### Example 3

### Formulation and Stability Studies

### Stability in Different Buffers

A stock solution of the antibody at a concentration of 1 and 10 mg/mL was created in the different buffers as shown in table 6.

TABLE 6

| | Trehalose | PS20 0.01% |
|---|---|---|
| Buffer | 100 mM | (wt./vol.) |
| 20 mM PBS pH 7.4± | − | − |
| 20 mM PBS pH 7.4 | + | − |
| 20 mM PBS pH 7.4 | + | + |
| 20 mM Histidine pH 6.0 | − | − |
| 20 mM Histidine pH 6.0 | + | − |
| 20 mM Histidine pH 6.0 | + | + |
| 50 mM Na Acetate pH 5. | − | − |
| 50 mM Na Acetate pH 5.6 | + | − |
| 20 mM Citrate, pH 5.0 | + | − |
| 20 mM Citrate, pH 5.0 | + | + |

Aliquots of 60 mL of the protein solution were placed into amber glass vials [with glass insert and volume capacity of 0.35 mL (ThermoFisher); vials caps (PTFE/Red Silicone septa) (Agilent) and incubated at a range of temperatures (40° C., 50° C., 55° C., and 60° C.) in a Mini Block Heater with Heated Lid (VWR) filled with Techne® Ceramic Bath Beads (1.5-2 mm size, VWR). Samples were collected after 24 hours incubation and analyzed by several HPLC techniques. The peaks on the chromatograms were analyzed to determine the percentage of mAb monomer present in solution.

### Controlled Temperature Storage Stability Studies

Accelerated stability studies were set up for the different bispecific Antibodies concentrations of 1 and 10 mg/mL in different buffer and aliquoted in 20 mL volumes in PCR tubes. The aliquots were incubated at 40° C. in a PCR thermocycler heating block for a period of 15 days. Aliquots were collected in time and analyzed by several HPLC techniques. The peaks on the chromatograms were analyzed to determine the percentage of mAb monomer present in solution.

### Freeze and Thaw Studies

To study the effect of the freeze-thaw cycles, aliquots of the antibodies at 1 and 10 mg/ml were placed for 48 h into a −80° C. freezer. After freezing, the samples were thawed and analyzed by several HPLC techniques. The peaks on the chromatograms were analyzed to determine the percentage of mAb monomer present in solution. The freeze-thawing procedure was performed 2 times.

Aliquots of 100 mL of the protein solutions into amber glass were fast frozen by immersion in subzero solvent (alcohol+CO₂(s)) bath and placed into a −80° C. freezer for a period of 3 days prior to thawing. Samples were thawed in one of two ways: either slowly at room temperature on the bench top (slow), or more quickly (fast) in a 37° C. incubator for a few minutes until completely thawed.

### Results

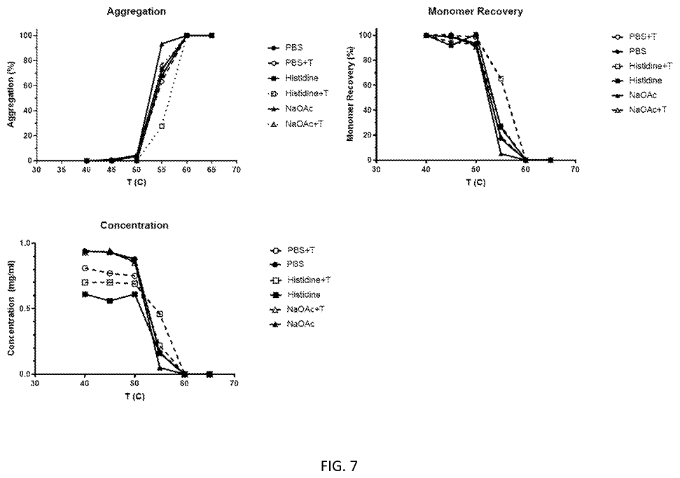

FIG. 7 presents the results of formulation studies performed with the ABCB1XCD47 KBisP1.1 (15D3 HC KK/KT14 HC DD/MRK16 LC) bispecific antibody. The aggregate concentration, monomer recovery and antibody concentration remained highly stable up to about 50-55° C.

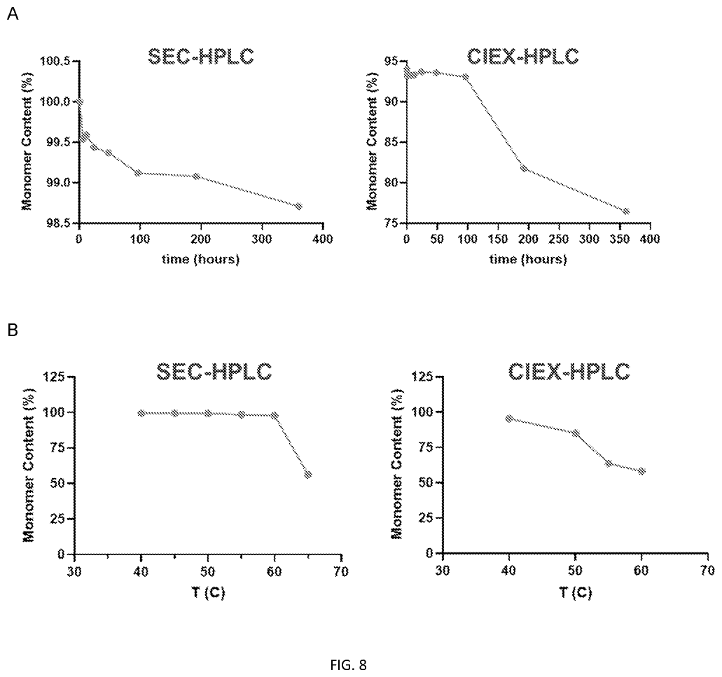

FIG. 8 presents long term stability and thermostability data for the ABCB1XCD47 bispecific antibody KBisP1.1 (15D3 HC KK/KT14 HC DD/MRK16 LC). Long term stability was assessed at 40°. Thermostability was tested at 1 mg/ml in 20 mM Histidine, pH 6.0+100 mM Trehalose between 40-65° C. The KBisP1.1 showed excellent long-term stability up to about 100 hours and thermostability up to about 55-60° C. under the conditions tested, as assessed by monomer content (%).

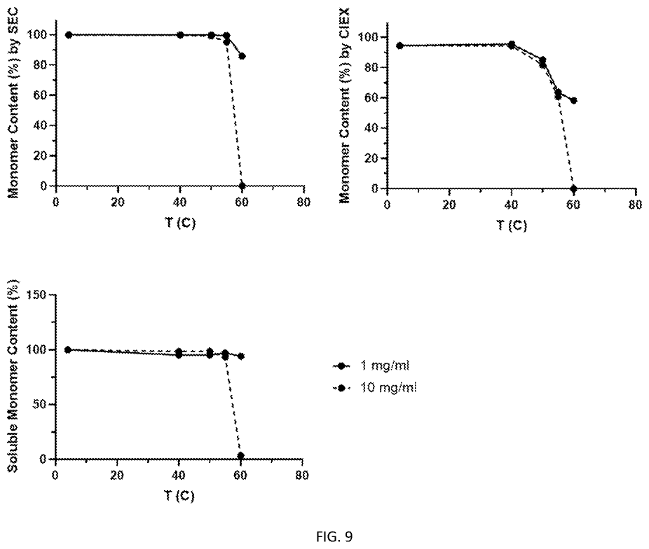

FIG. 9 presents further thermostability data for the ABCB1XCD47 bispecific antibody KBisP1.1 at 1 and 10 mg/ml in 20 mM Histidine, pH 6.0+100 mM Trehalose.

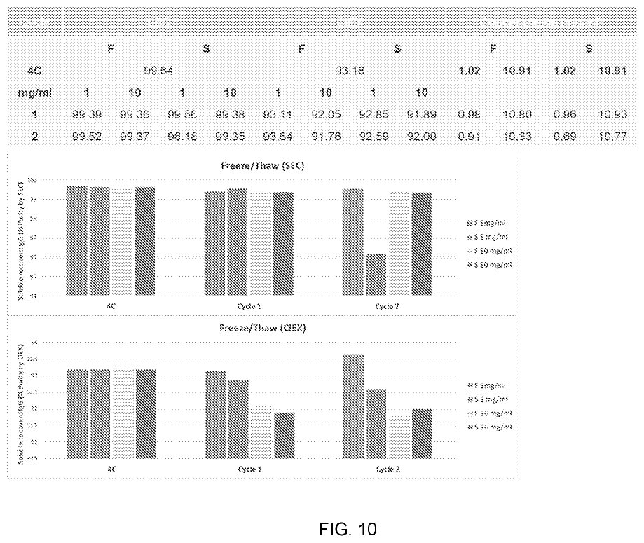

FIG. 10 presents the results of freeze-thaw stability studies with the ABCB1XCD47 KBisP1.1 bispecific antibody at 1 and 10 mg/ml in 20 mM Histidine, pH 6.0+100 mM Trehalose.

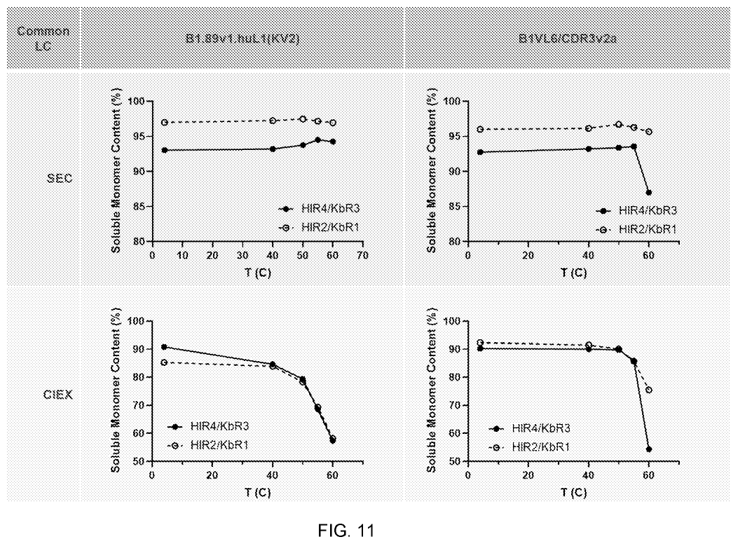

FIG. 11 presents thermostability results for bispecific ABCB1XCD47 antibodies, which have the 15D3 HIR2/KT14 KbR1 and 15D3 HIR3/KT14 KbR4 heavy chains paired with two different light chains, B1VL6/CDR3v2a and B1.89v1.huL1(KV2) at 1 mg/ml in 20 mM Histidine, pH 6.0+100 mM Trehalose. The tested bispecific antibodies with HIR2/KbR1 and HIR3/KbR4 Fc mutations, respectively show excellent thermostability up to about 55-60° C., as assessed by measuring the soluble monomer content (%).

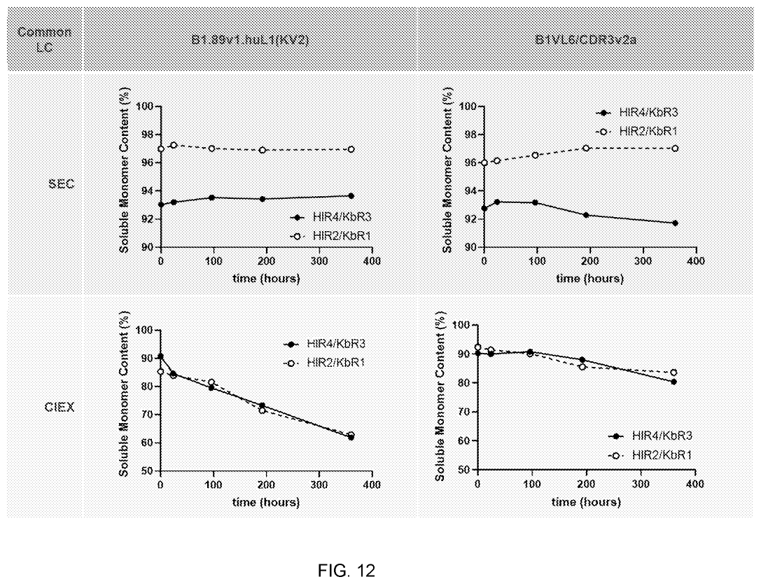

FIG. 12 presents long-term stability results for bispecific ABCB1XCD47 antibodies, which have the 15D3 HIR2/KT14 KbR1 and 15D3 HIR3/KT14 KbR4 heavy chains paired with two different light chains, B1VL6/CDR3v2a and B1.89v1.huL1(KV2) at 1 mg/ml in 20 mM Histidine, pH 6.0+100 mM Trehalose. The tested bispecific antibodies with HIR2/KbR1 and HIR3/KbR4 Fc mutations, respectively, show excellent long-term stability up to about 400 hours.

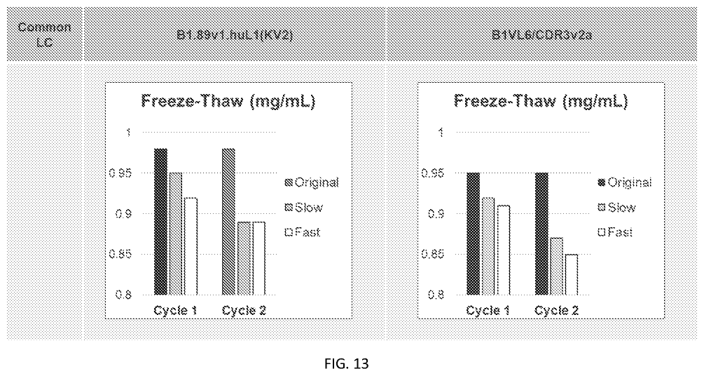

FIG. 13 presents the freeze/thaw stability results at 1 mg/ml of two bispecific antibodies having the 15D3 HIR2/KT14 KbR1 and 15D3 HIR3/Kt14 KbR4 heavy chains paired with two different light chains, B1VL6/CDR3v2a and B1.89v1.huL1(KV2), respectively in 20 mM Histidine, pH 6.0+10 mM Trehalose.

Thermostability and long-term accelerated stability assays confirmed that the bispecific antibodies produced with the HIRX/KbRY engineered heavy chains are highly stable and have excellent biophysical characteristics comparable to the corresponding KKDD and knobs-into-holes bispecific antibodies. The bispecific antibodies comprising the HIRX/KbRY Fc mutations were stable in various formulations at different concentrations and could undergo to multiple freeze-thaw cycles without any stability issues.

### Example 4

### Binding Studies—ELISA

### ELISA Assay to Detect Binding to Human CD47

Binding specificity of the mAbs, Fab and Bispecific IgG1 targeting CD47 was tested by ELISA using mouse CD47-Fc fusion protein (R&D systems). Briefly, microtiter plates were coated with 50 μL of recombinant extracellular domain of human CD47-Fc fusion protein at 0.5 pg/ml in PBS, and then blocked with 100 μl of 0.4% BSA in PBS. Dilutions of the different antibody formats were added in 1/3 sequential dilutions to each well and incubated for 1 hour at room temperature. The 5F9 known anti-CD47 antibody was used as a positive control, and human IgG1 was used as an isotype control. Plates were subsequently washed three times with PBS/Tween and then incubated with HRP-conjugated donkey anti-human constant specific secondary reagent for 1 hour at room temperature. After washing, plates were developed with HRP substrate. The reaction was stopped with 2M H₂SO₄, and OD was measured at 520 nm.

### Results

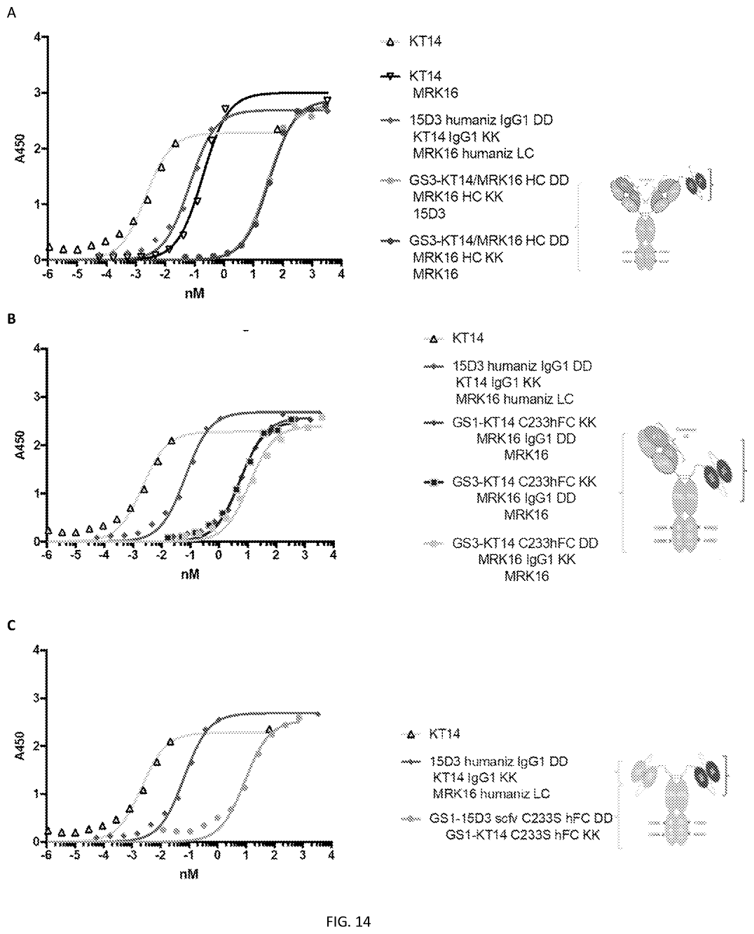

FIG. 14 shows the binding of different formats of ABCB1XCD47 bispecific antibodies to the extracellular domain (ECD) of huCD47 tested by ELISA as described above. The binding of the different formats was compared to the binding of 5F9 and the 15D3 hmz DD/KT14 KK/MRK16 hmz bispecific antibody in the IgG1 format. As evidenced by the results, all antibodies tested in all formats showed good binding to the human CD47 target.

The binding of the different formats of bispecific antibodies to the extracellular domain of human CD47 shows the potential to modulate the affinities to the selected TAA targets using several antibody-based bi- and multi-specific formats. For example, affinity can be modulated using an antibody format with a different valency for a target and/or combining different light and heavy chain pairs.

Modulating affinity can enable a minimal off-target binding thereby reducing potential toxicity and an efficacious, tumor-specific binding of therapeutic antibodies to cells where both targets are overexpressed.

### Example 5

### Cell Binding Studies, Efflux Blockade, Cell Sensitization to Chemotherapeutics

### Reagent Cell Lines Used to Test: Binding, Efflux Blockade, Cell Sensitization to Chemotherapeutics

HEK 293T and N6ADR cell lines were obtained from the American Type Culture Collection. SA-MES/DX5 was obtained from the European Collection of Authenticated Cell Cultures (ECACC) through the United States distributor Sigma-Aldrich).

N6/ADR cells are also referred to as NALM6/ADR cells. All cell lines and further cell lines derived from them were maintained in RPMI 1640 or DMEM supplemented with up to 10% fetal bovine serum (Avantor Seradigm), nonessential amino acids, and 2 mmol/L L-glutamine at 37° C. and 5% C02 in a humidified incubator (unless otherwise indicated). Cells were used as supplied or were modified to overexpress (Ox) Pgp or were subject to having Pgp expression knocked down (KD) with lentiviral-mediated short hairpin RNA or knock out (KO) of the functional CD47 gene by CRISPR/Cas-mediated knock out technology essentially as described (Cong, L. et al. (2013) Science 339, 819-823).

For vincristine and paclitaxel IC50 determination, cells were plated in normal growth medium and incubated overnight. Paclitaxel or vincristine (Sigma) were added in a dilution series and any modulators were added within ranges of 0 to 500 μM/L. Cell viability was measured 72 h later using the Celltiter-Glo Luminescent Cell Viability Assay (Promega). The concentration of drug resulting in 50% inhibition of cell viability (IC50) was calculated from a multi-parameter curve analysis (GraphPad Prism software GraphPad Software, Inc.) and was determined from a minimum of 2 repeats. Cell lines that did not show 50% reduction in cell viability in response to drug and/or modulator treatment in the majority of experiments conducted were considered to not have reached an IC50 by definition and are listed as having an IC50 of >1000 nmol/L for paclitaxel or the drug/modulator combination under study.

### Generation of a Stable ABCB1 Overexpressing (Ox) Cell Lines

In order to characterize both binding and in vitro efficacy, a cell line that stably over-expressed ABCB1was developed. Adherent 293T naïve cells obtained from American Type Culture Collection (ATCC) were utilized. As characterized by flow cytometry using a commercially available ABCB1 antibody (Biolegend, clone 4E3.16), this cell line expresses ABCB1 endogenously at a low to moderate degree on the cell surface. 293T naïve cells were transfected with ABCB1 using Polyplus PElpro reagent. Three days after transfection, cells were put under selection using a Hygromycin B solution (Millipore Sigma). Fourteen days after continuous Hygromycin B selection 293T cells were evaluated for ABCB1 cell surface expression. To ensure non-transfected cells would not expand in future cultures, a bulk sort using fluorescent activated cell sorting (FACS) of ABCB1 positive 293T cells was performed using a FACSArial (BD Biosciences). The bulk sorted 293T ABCB1 over-expressing cells were expanded and ABCB1 over-expression was subsequently re-confirmed. Similar methods were used to generate ABCB1 over-expressing cells from 293T-CD47 Knock-Out cells generated as described herein.

### Generation of CRISPR KO Cell Lines

To construct gene knockout cell lines, the host cells were first cultured in DMEM (Dulbecco's Modified Eagle's Medium, Gibco, Grand Island, N.Y., USA) supplemented with 10% (v/v) FBS, and glutamine via adhesion culture. Cells were cultivated at 37° C. with 5% C02 at saturated humidity. Transfection of cells was performed by lipid-based transfection using the CRISPRMax reagent (ThermoFisher) according to the manufacturer's protocol. Briefly, one day prior to transfection, adherent cells were plated onto 96-well plates at 0.2×105 cells per well. On the day of transfection, a solution of GeneArt Platinum Cas9 protein, gRNA and transfecting reagent was added to cells. 72 h post-transfection, single cells were selected by a limiting dilution method and cell culture was continued for 2 weeks in a 96 well plate format prior to subsequent testing. The design of the gRNAs was performed using the online CHOPCHOP web tool for selecting target sites for CRISPR/Cas9, CRISPR/Cpf1 or TALEN-directed mutagenesis.3,4. All the designed gRNAs were chemically synthesized by ThermoFisher.

### Efflux Inhibition

HEK 293T cells over-expressing human ABCB1 were washed several times and aliquoted into 96-well plates as 50 I aliquots/well at a cell density of 2×10⁶ cells per ml in phenol red-free Dulbecco's Modified Eagle's Medium (DMEM). Cells were mixed with 50 I aliquots of antibodies and 10 1 6 μM Calcein AM, and incubated for 2 h at 37° C. The cells were then washed twice and finally resuspended in 200 μl PBS. Calcein AM fluorescence retained in the cells was measured by flow cytometry. Efflux blockade was measure using the Multidrug Resistance Direct Dye Efflux Assay (Chemicon) following the manufacturer's protocol.

### Cytotoxicity Screenin—Assay

The effect of antibodies on vincristine-induced cytotoxicity was evaluated using the drug-resistant N6/ADR cell line. In this assay, vincristine was serially diluted, while the ABCB1 x CD47 BsAbs (or isotype control antibodies) were used at a uniform, saturating concentration. The assay readout was vincristine IC₅₀ in the presence or absence of antibodies. Inhibition of ABCB1 should result in a lower vincristine IC₅₀.

The assay was performed as follows: cells were plated in 0.05 mL of assay media (RPMI-1640+10% FBS) at 5000 cells/well in white, flat bottom 96-well tissue culture plates and incubated overnight at 37° C., 5% CO₂. The following day vincristine was prepared at 2X final assay concentration by 1:5 or 1:10 serial dilutions in screening assay media containing test antibodies or control antibodies at 100 μg/mL (2X final concentration), or valspodar, a small molecule ABCB1 inhibitor, at 7 pM (2X final concentration). An equivalent volume (0.05 mL) of the vincristine/antibody mixture was added to the N6/ADR cells and incubated at 37° C., in 5% CO₂.

After 72 hours, plates were removed from the incubator and allowed to equilibrate to room temperature. Viability was assessed using the Promega® CellTiter-Glo® Luminescent Cell Viability Assay according to the manufacturer's recommended protocol. Luminescence was measured on a Molecular Devices® FlexStation® 3 Multi-Mode Microplate Reader, and data were analyzed using GraphPad Prism software to calculate vincristine IC50 which is the concentration of drug (vincristine) that reduces a response (cell growth) by 50%.

### Cytotoxicity Potency Assay

The relative potency of the samples was evaluated using N6/ADR cells. These cells expressed both ABCB1 and CD47 with the expression of CD47 ˜40-fold higher than ABCB1 as determined by staining with R-phycoerythrin (R-PE) conjugated, commercially available antibodies against ABCB1 (UIC2, Biolegend) and CD47 (CC2C6, Biolegend). This assay also examined the effect of antibodies on vincristine-induced cytotoxicity using the drug-resistant cell line, N6/ADR. However, in this assay format the concentration of vincristine was held constant while the ABCB1 x CD47 BsAbs (or isotype control antibodies) were serially diluted. This assay measured antibody IC₅₀. Lower IC₅₀ meant higher antibody potency.

The potency assay was performed as follows. Cells were plated in 0.05 mL of assay media at 5000 cells/well in white flat bottom 96-well tissue culture plates and incubated overnight at 37° C., 5% CO₂. The following day antibodies were serially diluted 1:2 or 1:3 from 200 g/mL (2× final concentration) in cytotoxicity potency assay media [RPMI-1640+10% FBS containing 0.01 mM vincristine (2× N6/ADR IC50)]. An equivalent volume (0.05 mL) diluted antibody/vincristine mixture was transferred to the N6/ADR cells and incubated at 37° C., in 5% CO². After 72 hours, plates were removed from the incubator and allowed to equilibrate to room temperature. Viability was assessed using the Promega® CellTiter-Glo® Luminescent Cell Viability Assay according to the manufacturer's recommended protocol. Luminescence was measured on a Molecular Devices® FlexStation®3 Multi-Mode Microplate Reader, and data were analyzed using GraphPad Prism software to calculate the antibody IC50 on N6/ADR cells in the presence of 5 nM (final) vincristine.

### Receptor Occupancy Assay

Receptor Occupancy (RO) is a measure of the binding of a biotherapeutic to its cellular target(s). Common detection reagents include the drug itself and one or more antibodies binding to the same epitope as the drug. In the present experiment, anti-ABCB1 and anti-CD47 antibodies binding the respective epitopes of the corresponding ABCB1xCD47 bispecific antibodies were used, using the A2780ADR (ovarian) drug resistant cell line. A schematic of the Receptor Occupancy assay is illustrated in FIG. 21. Antibodies were prepared by 1:3 serial dilutions in flow cytometry buffer starting from 200 μg/mL (2× final concentration). 50 μL of diluted antibodies were transferred to the cell mixture and the plates incubated at 4° C. for 30 minutes. Following two washes with flow cytometry buffer, cells were resuspended in 50 L of flow cytometry buffer containing the detection antibody PE conjugated anti-human IgG Fc gamma-specific (Jackson #709-116-098) and incubated at 4° C. for 15 minutes. Following two washes with flow cytometry buffer, cells were resuspended in 100 L of flow cytometry buffer and separated in two samples. One was incubated with the secondary detection antibodies Alexa 647-conjugated anti-hCD47 CC2C6 antibody and the other one with Alexa 647-conjugated anti-ABCB1 MRK16 antibody. The two samples were incubated at 4° C. for 30 minutes. Following two washes with flow cytometry buffer, the cells from each sample were resuspended in flow cytometry buffer, plus DAPI, to gate out dead cells, and analyzed using an Attune NXT (ThermoFisher Scientific) flow cytometer.

### Results

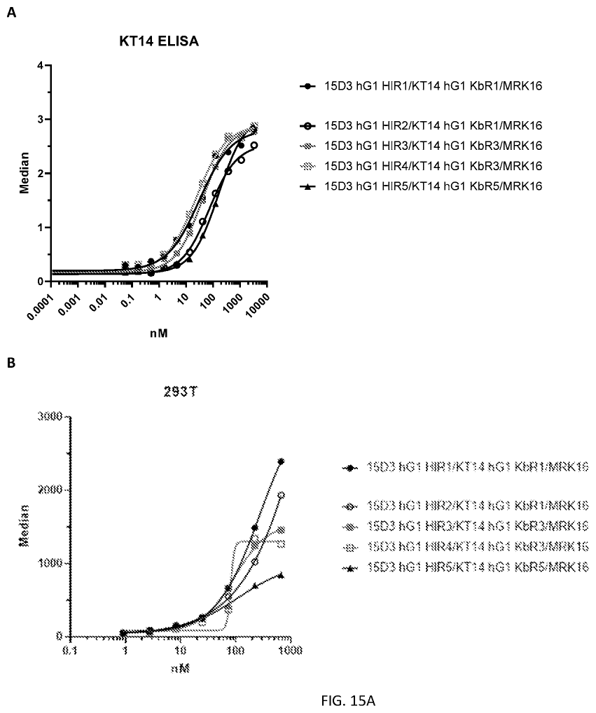

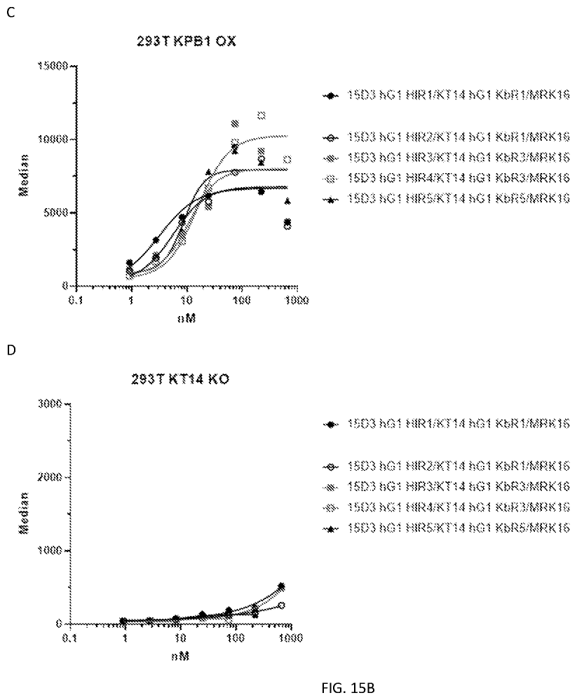

FIGS. 15A and 15B shows the binding of ABCB1XCD47 bispecific antibodies with KiH and modified KiH Fc region, tested by FACS to HEK 293T naïve cell line expressing high level of hCD47 and moderately low level of ABCB1 (B; ABCB1 overexpressing HEK 293T cells (C); and HEK 293T CD47 KO cells (D). The binding to the extracellular domain (CD) of huCD47 was tested by ELISA (A). All tested bispecific antibodies have the same VH and VL sequences as shown.

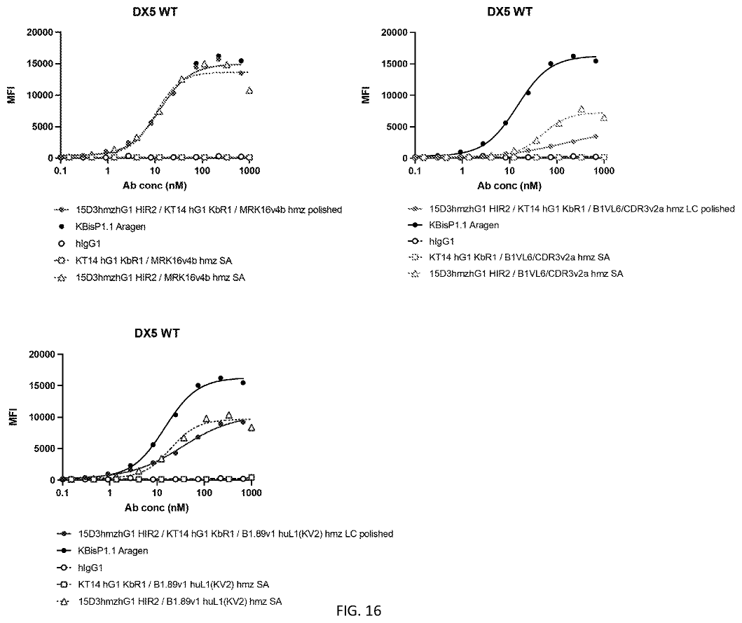

FIG. 16 shows the binding of bispecific, humanized ABCB1XCD47 antibodies 15D3hmzG1 HIR1/KT14 hG1 KbR1/MRK16v4b hmz LC polished, 15D3hmzG1 HIR1/KT14 hG1 kbR1/B1VL6/CDR3v2a hmz LC polished, and 15D3hmzhG1 HIR1/KT14 hG1 KbR1/B1.89.v1 huL1(KV2) hmz LC polished to wild-type DX5 (DX5 WT) cells, relative to bispecific ABCB1XCD47 antibody KBisP1.1, and anti-ABCB1 and anti-CD47 mono arm antibodies, having the same light chain as the corresponding bispecific antibodies. The mono arm antibodies each have only one binding valency for the target. The wild-type DX5 (DX5 WT) cells express both ABCB1 and CD47 antigens. The Kd constants for KT14 (ABCB1) binding calculated by Octet are shown in Table 7.

TABLE 7

| | | |
|---|---|---|
| 1 | 15D3hmzhG1 HIR2/KT14 hG1 kbR1/MRK16v4b hmz LC | 13.1 |
| 2 | 15D3hmzhG1 HIR2/KT14 hG1 kbR1/B1VL6/CDR3v2a | 29.9 |
| | hmz LC | |
| 3 | 15D3hmzhG1 HIR2/KT14 hG1 kbR1/B1.89v1 huL1(KV2) | 10.6 |
| | hmz LC | |

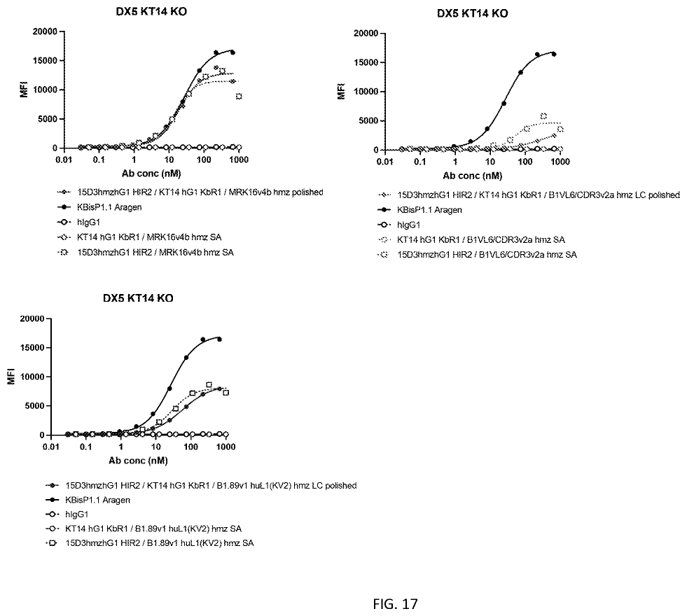

FIG. 17 shows the binding of bispecific, humanized ABCB1XCD47 antibodies 15D3hmzG1 HIR1/KT14 hG1 KbR1/MRK16v4b hmz LC polished, 15D3hmzG1 HIR1/KT14 hG1 kbR1/B1VL6/CDR3v2a hmz LC polished, and 15D3hmzhG1 HIR1/KT14 hG1 KbR1/B1.89.v1 huL1(KV2) hmz LC polished to DX5 KT14 KO cells, relative to bispecific ABCB1XCD47 antibody KBisP1.1, and anti-ABCB1 and anti-CD47 mono arm antibodies, having the same light chain as the corresponding bispecific antibodies. The mono arm antibodies only have one binding valency for the target. The DX5 KT14 KO cells do not express the CD47 antigen.

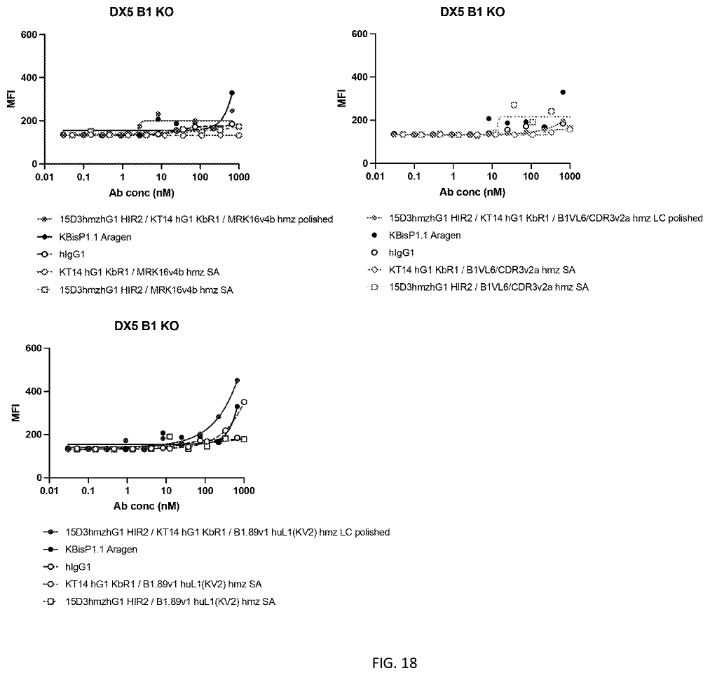

FIG. 18 shows the binding of bispecific, humanized ABCB1XCD47 antibodies 15D3hmzG1 HIR1/KT14 hG1 KbR1/MRK16v4b hmz LC polished, 15D3hmzG1 HIR1/KT14 hG1 kbR1/B1VL6/CDR3v2a hams LC polished, and 15D3hmzhG1 HIR1/KT14 hG1 KbR1/B1.89.v1 huL1(KV2) hmz LC polished to DX5 ABCB1 KO (B1 KO) cells, relative to bispecific ABCB1XCD47 antibody KBisP1.1, and anti-ABCB1 and anti-CD47 mono arm antibodies, having the same light chain as the corresponding bispecific antibodies. The mono arm antibodies each have only one binding valency for the target. The DX5 ABCB1 KO cells do not express the ABCB1 antigen.

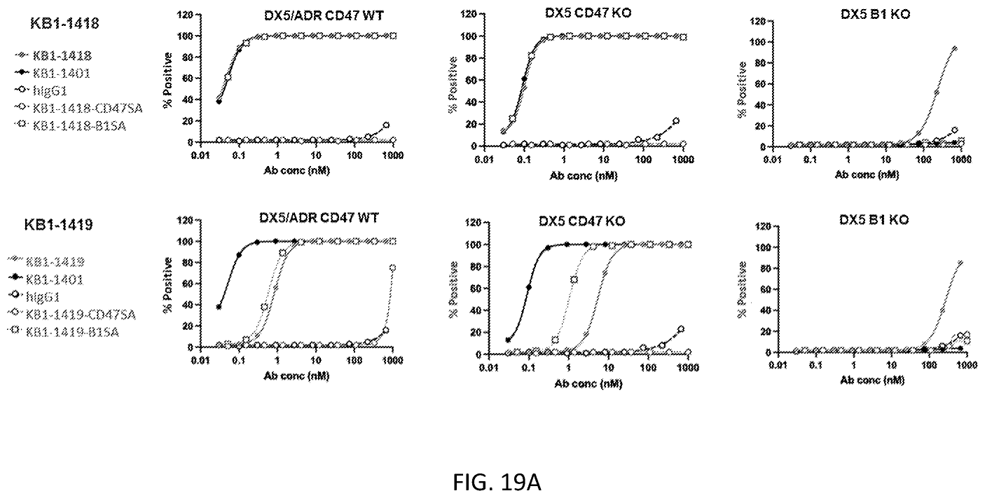

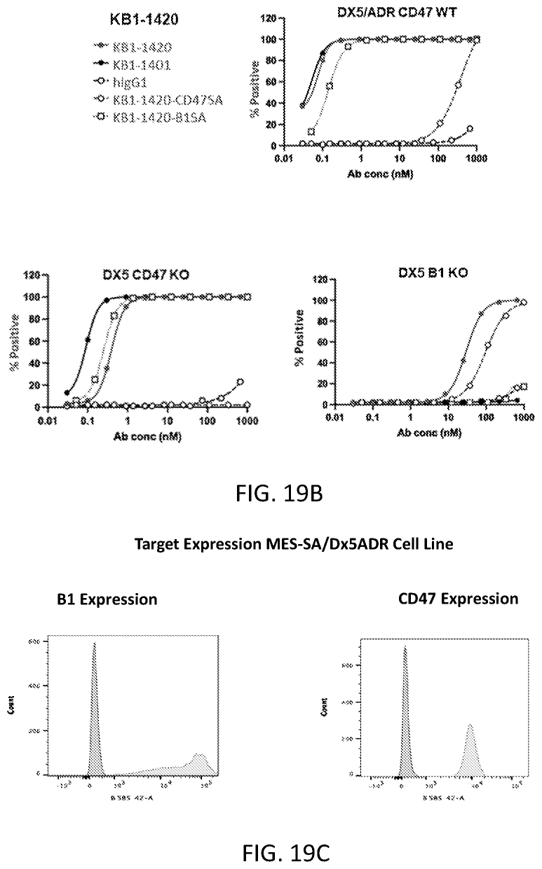

FIGS. 19A-19C show the binding of bispecific humanized ABCB1XCD47 antibodies KB1-1418, KB1-1419 and KB1-1420 to wild-type Adriamycin-resistant DX5 cells, CD4 KO DX5 cells and ABCB1 KO (B1 KO) DX5 cells. The results show that the KB1-1418, KB1-1419 and KB1-1420 specifically bind to ABCB1 and CD47 antigens expressed on cancer cell surface.

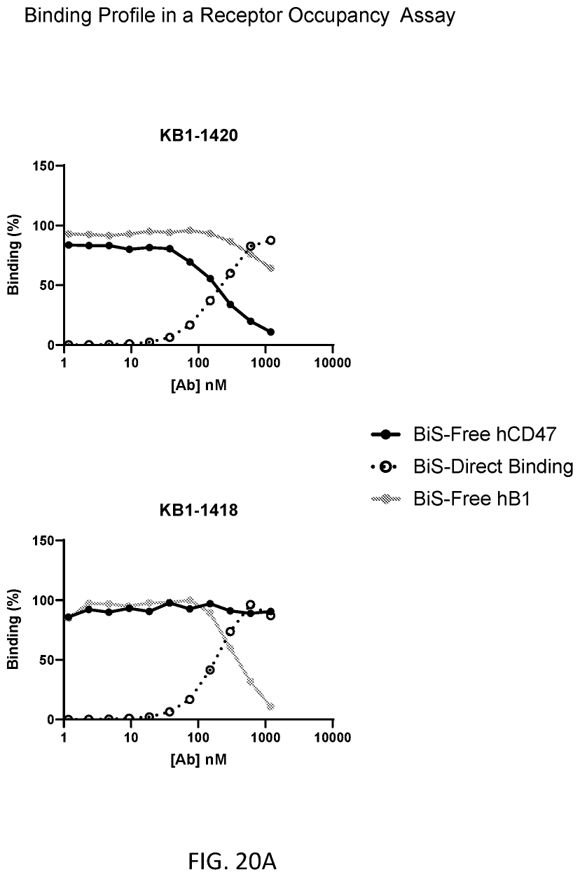

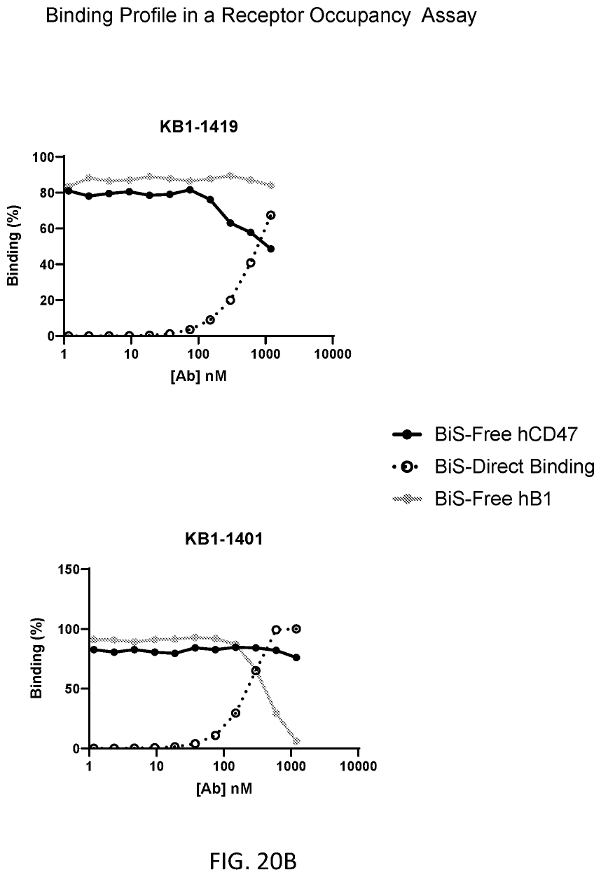

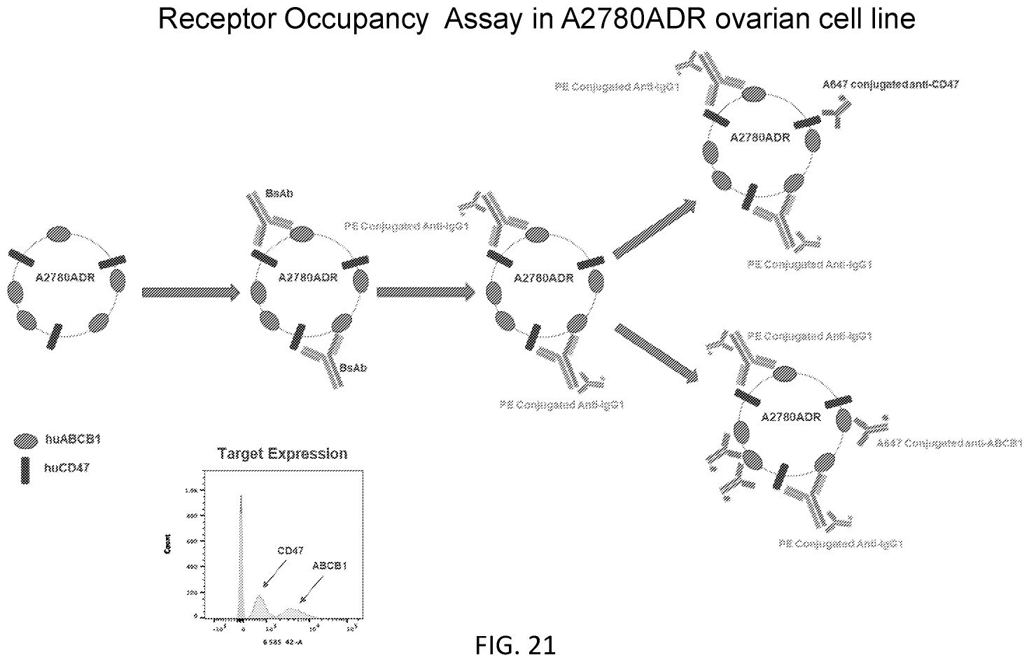

FIGS. 20A-20B show the binding profiles of humanized bispecific ABCB1XCD47 antibodies KB1-1418, KB1-1419, and KB1-1420 in the receptor occupancy assay described above, which shows the relative binding of the tested antibodies in the multidrug resistant ovarian adenocarcinoma tumor cell line A2780ADR.

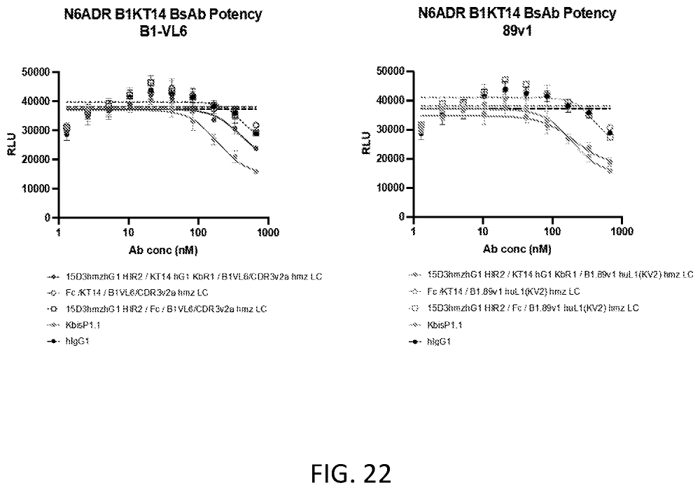

The schematic illustration of the receptor occupancy assay is shown in FIG. 21. FIG. 22 presents the results of potency assays in ABCB1 expressing N6ADR cell lines performed with the ABCB1XCD47 bispecific antibodies 15D3hmzG1 HIR2/KT14 hG1 KbR1/B1VL6/CDR3v2a hmz LC and 15D3hmzG1 HIR2/KT14hG1 KbR1/B1.89.v1 huL1(KV2) hmz LC relative to bispecific ABCB1XCD47 antibody KBisP1.1, and anti-ABCB1 and anti-CD47 mono arm antibodies, having the same light chain as the corresponding bispecific antibodies.

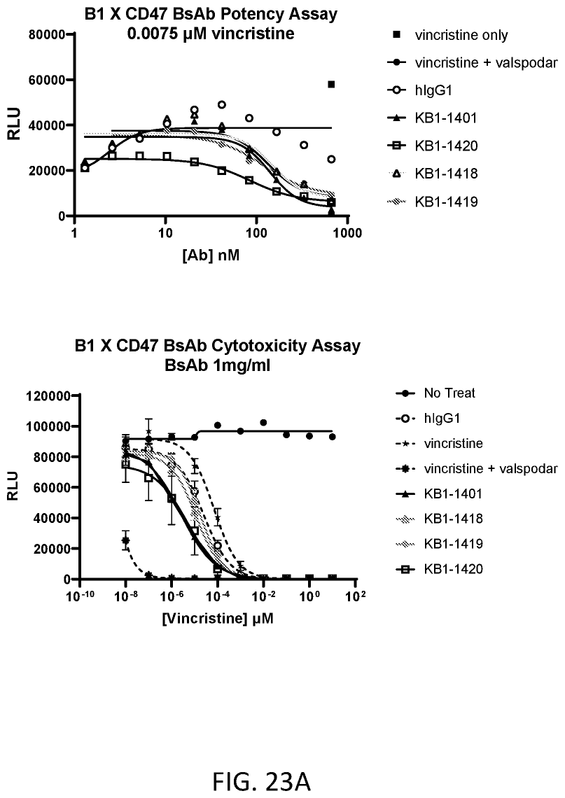

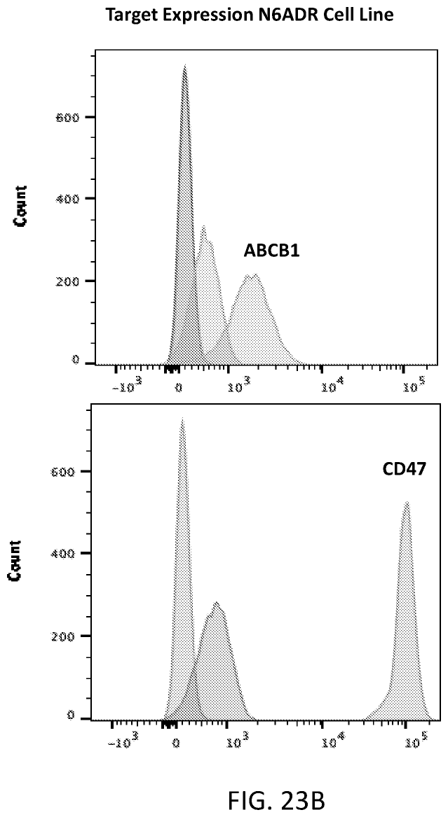

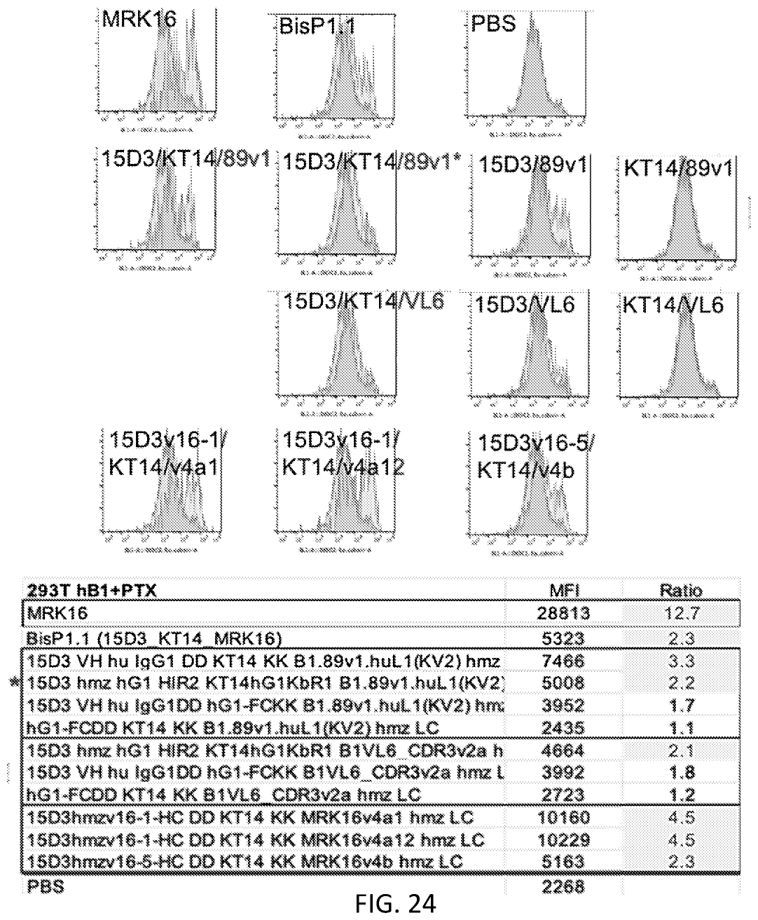

FIGS. 23A-23B present in vitro potency data in a N6ADR drug resistant cell-based leukemia assay demonstrating that the tested humanized bispecific ABCB1× CD47 antibodies KB1-1418, KB1-1419 and KB1-1420 show significant in vitro potency (cell killing) in this cytotoxicity screening assay.

FIG. 24 shows that the tested bispecific ABCB1XCD47 antibodies inhibit efflux in Paclitaxel treated 293T hB1 cells overexpressing ABCB1 antigen.

Bispecific antibodies with the same VH and VL regions produced with the heavy chain HIRX/KbRY Fc mutations bind with similar affinities to the ABCB1 and CD47 antigens as indicated by the ELISA results and the FACS data on the different HEK293 cell lines.

Three different light chains were paired with the 15D3 humanized VH and the KT14 VH using the HIR2/KbR1 heavy chain format. The binding of the produced ABCB1XCD47 antibodies was compared to the binding of the bispecific antibody KBisP1.1, and anti-ABCB1 and anti-CD47 mono arm antibodies to wild-type DX5 and ABCB1 and CD47 Knock out DX5 cells. The HIR2/KbR1 BsAb with the MRK16v4b LC binds better to the ABCB1 antigen while the Kd calculated from Octet binding data showed a similar affinity for the CD47 antigen. This confirms that it is possible to modulate affinity by using different engineered VL domains in the light chain.

N6/ADR cells were tested as target cells for development of a potency assay to provide improved functional discrimination between antibodies. In this assay, 15D3hmz HIR2/KT14 KbR1/B1.89.v1 huL1(KV2) bispecific shows a potency similar to that of the bispecific antibody KBisP1.1 increasing cytotoxicity. As expected the anti-CD47 mono arm antibody (Fc/KT14/B1.89.v1 huL1(KV2) shows no effect while both the anti-ABCB1 mono arm antibodies (15D3 hmz HIR2/Fc/B1.89.v1 huL1(KV2) and 15D3 hmz HIR2/Fc/B1VL6/CDR3v2a hmz) show a lesser potency than the corresponding bispecific antibodies even if having the same valency (one binding arm) suggesting a possible synergism or co-operative engagement due to the binding to both antigens.

The tested bispecific antibodies were able to inhibit efflux in Paclitaxel treated HEK293 overexpressing ABCB1 cells to different extents. 15D3hmz/KT14/B1.89.v1 huL1(KV2) bispecific antibodies in both KKDD and HIRX/KbRY format similarly inhibit efflux in an extent comparable to that of the 15D3 DD/FC KK/B11.89.v1 huL1(KV2) mono arm antibody. No efflux inhibition was observed with the KT14 mono arm antibodies pairing the KT14 HC with B1.89.v1 huL1(KV2) or B1 VL6/CDR3v2a light chains.

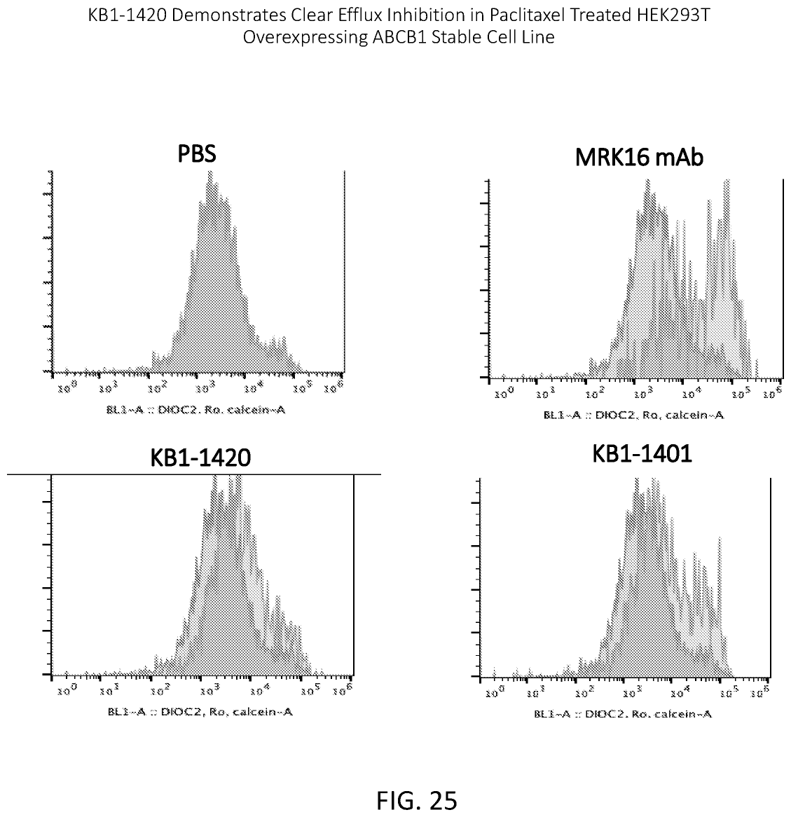

FIG. 25 demonstrates clear efflux inhibition by the ABCB1XCD47 bispecific antibody KB1-1420 in paclitaxel-treated ABCB1 overexpressing HEK293T stable cell line.

### Example 6

### Antibody Binding to Human and Cynomolgus Red Blood Cells (RBC)

### Antibody Binding to Human and Cynomolgus Red Blood Cells (RBC)

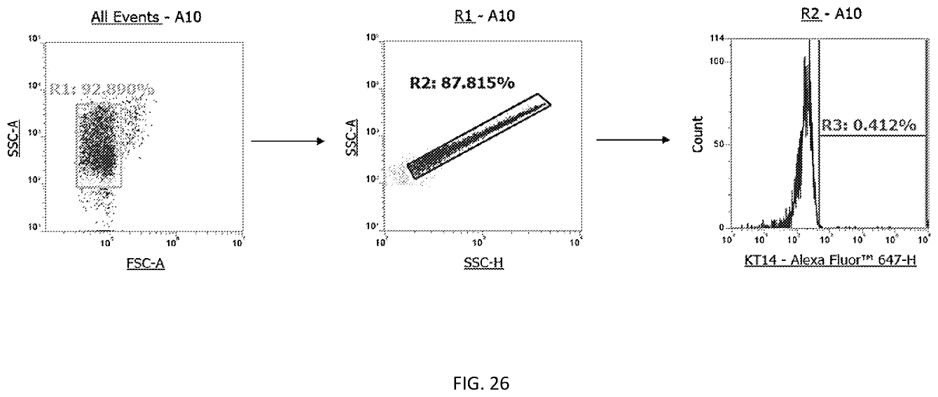

Antibody binding to human and cynomolgus RBC was assessed by flow cytometry. Normal human and cynomolgus whole blood was obtained from BiolVT. RBC were prepared by centrifugation of whole blood aliquots (0.5 mL) for 5 min at 500xg. Supernatant (plasma) was removed, and RBC washed 3 times in D-PBS+1 mM EDTA. After the final wash, the RBC were resuspended in D-PBS+1 mM EDTA to a final volume of 0.5 mL. Washed RBC were then diluted in flow cytometry buffer (D-PBS+2% FBS+0.05% sodium azide) to 2% and dispensed at 50 pL/well into 96-well plates. An equivalent volume (50 pL) of antibody serially diluted in flow cytometry buffer to 2× final was added to the RBC, and the mixture incubated for 1 hour at room temperature. Next, RBC were washed 3 times with flow cytometry buffer and resuspended in 50 μL of Alexa Fluor®647 conjugated Fab fragment goat anti-human IgG diluted 1:200 in flow cytometry buffer. After 20 minutes at room temperature RBC were washed twice, resuspended in flow cytometry buffer and analyzed using an Invitrogen™ Attune™ NXT flow cytometer. FIG. 26 shows the gating strategy for RBC staining. Left panel, RBC are gated (R1) based on forward and side scatter. Middle panel, a second gate (R2) is applied to eliminate any remaining doublets or aggregates. Right panel, fluorescence of the R2 gated cells is assessed and a final gate (R3) is set using unstained RBC. Cells falling in the R3 gate are defined as “positive” for binding and reported as the percent (of R2 gated cells) that fall in the R3 gate. The results are presented at percent positive which represents the percent of RBC in the R3 gate.

### Results

The results of testing binding of various humanized bispecific ABCB1XCD47 antibodies to cynomolgus red blood cells are shown in the following Table 8.

TABLE 8

| Binding to cynomolgus Red Blood Cells | % Positive | | | |
|---|---|---|---|---|
| Test Article | Cy744 | Cy745 | Cy746 | Cy747 |
| IgG1 | 0.1 | 0.9 | 1.2 | 0.5 |
| KT14 | 99.0 | 98.8 | 99.4 | 99.0 |
| 15D3hG1HIR2/KT14hG1KbR1/B1.89v1.huL1(KV2) hmz LC | 7.9 | 10.6 | 57.7 | 74.5 |
| 15D3VHhuIgG1DD/KT14KK/B1.89v1.huL1(KV2)hmzLC | 0.7 | 2.2 | 15.1 | 26.5 |
| 15D3DDIgG1/KT14VHKKIgG1/89v1VL in huIgG1 LC K | 0.6 | 1.2 | 1.7 | 6.5 |
| 15D3hmzhG1 HIR2/Fc/B1.89v1 huL1(KV2) hmz LC | 0.3 | 0.6 | 0.8 | 4.0 |
| Fc/KT14/B1.89v1 huL1(KV2) hmz LC | 0.9 | 1.7 | 5.0 | 18.8 |
| 15D3hmz16-1/KT14/MRK16v4a1 | 0.4 | 0.4 | 1.5 | 5.7 |
| 15D3hmz16-1/KT14/MRK16v4a12 | 0.3 | 0.2 | 0.6 | 3.4 |
| KbisP1.1 | 0.5 | 0.7 | 2.0 | 3.7 |

The results of testing the binding of various humanized bispecific ABCB1XCD47 antibodies to human red blood cells are shown in the following Table 9.

TABLE 9

| Binding to human Red Blood Cells | % positive | | | |
|---|---|---|---|---|
| Test Article | D834 | D835 | D836 | D837 |
| IgG1 | 0.3 | 0.4 | 0.2 | 0.4 |
| KT14 | 99.1 | 98.9 | 98.9 | 99.1 |
| 15D3hG1HIR2/KT14hG1KbR1/B1.89v1.huL1(KV2) hmz LC | 2.0 | 1.4 | 1.1 | 1.6 |
| 15D3VHhuIgG1DD/KT14KK/B1.89v1.huL1(KV2)hmzLC | 0.7 | 0.4 | 0.2 | 0.4 |
| 15D3DDIgG1/KT14VHKKIgG1/89v1VL in huIgG1 LC K | 0.3 | 0.2 | 0.2 | 0.2 |
| 15D3hmzhG1 HIR2/Fc/B1.89v1 huL1(KV2) hmz LC | 0.2 | 0.2 | 0.1 | 0.1 |
| Fc/KT14/B1.89v1 huL1(KV2) hmz LC | 0.4 | 0.1 | 0.2 | 0.3 |
| 15D3hmz16-1/KT14/MRK16v4a1 | 2.1 | 1.5 | 0.2 | 0.4 |
| 15D3hmz16-1/KT14/MRK16v4a12 | 0.4 | 0.2 | 0.3 | 0.3 |
| KbisP1.1 | 0.4 | 0.4 | 0.3 | 0.5 |

The 5F9 KT14 mAb binds to red blood cells which is to be expected as CD47 is involved in maintaining the balance of red blood cells in the body. No binding is observed with KBisP1.1, the 15D3hmz/KT14/B1.89.v1 huL1(KV2) bispecific antibodies in both KKDD and HIRX/KbRY formats and the mono-arm antibody Fc/KT14/B11.89v1 huL1(KV2) hmz. All produced Bispecific antibodies show reduced ability to bind to red blood cells suggesting that they could selectively bind tumor cells expressing both antigens without binding to red blood cells.

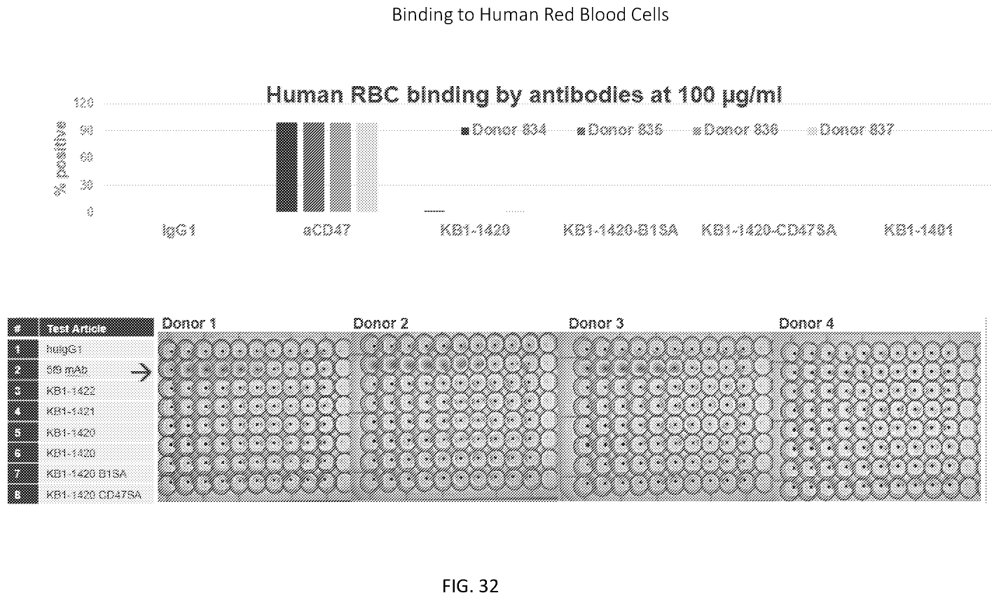

FIG. 32 further shows that bispecific humanized ABCB1XCD47 antibody KB1-1420 exhibits negligible binding to human red blood cells compared to anti-CD47 monoclonal antibody 5F9.

### Example 7

### In Vivo Studies

### Test Article Preparation for In Vivo Studies

The Bispecific Antibodies were produced in house and formulated in PBS. Human IgG1 isotype control was purchased from BioXcell and is formulated in PBS.

Paclitaxel (European Pharmacopoeia Reference Standard) was purchased from Sigma-Aldrich. A 50 mg/ml paclitaxel solution of was prepared in absolute ethanol and then diluted 1:2 with an equal volume of Kolliphor (Sigma-Aldrich). Prior to dosing mice, the paclitaxel/ethanol/Kolliphor solution was diluted 1:8 in PBS.

### In Vivo Anti-Tumor Effects on MES-SA/Dx5 Multidrup Resistance Model.

MES-SA/Dx5 cells were grown in RPMI-1640 supplemented with 10% FBS and 100 nM doxorubicin. Prior to implant cells were washed three times with D-PBS and resuspended in the same. Resuspended cells were mixed with an equal volume of Matrigel and xenografts were established by sub-cutaneous injection of 0.5×10⁶ tumor cells into 5-8-week-old female athymic nude mice. Tumors were measured three times a week using a calibrated digital caliper, and tumor volumes (TV) were calculated as per the formula: TV=/*L*S*S, where L is the long axis and s is the short axis of the tumor. Once tumors reached 100-150 mm³, mice were randomized into twelve groups of five mice each-(i) Control isotype IgG1 3 mg/kg, (ii) Control isotype IgG1 3 mg/kg and 20 mg/kg of paclitaxel, (iii) KNJYBisP1.1 3 mg/kg, (iv) KNJYBisP1.1 3 mg/kg and 20 mg/kg of paclitaxel, (v and vi) 15D3 HIR2/KT14 KbR1/B1.89v1.huL1(KV2) Bispecific 3 mg/kg, (vii and viii) 15D3 HIR2/KT14 KbR1/B1.89v1.huL1(KV2) Bispecific 3 mg/kg and 20 mg/kg of paclitaxel, (ix) 15D3hmz16-1 KK/KT14 DD/MRK16v4a1 Bispecific 3 mg/kg, (x) 15D3hmz16-1 KK/KT14 DD/MRK16v4a1 Bispecific 3 mg/kg and 20 mg/kg of paclitaxel, (xi) 15D3 HIR2/KT14 KbR1/B1VL6/CDR3v2a Bispecific 3 mg/kg, (xii) 15D3 HIR2/KT14 KbR1/B1VL6/CDR3v2a Bispecific 3 mg/kg and 20 mg/kg of paclitaxel.

Antibody and paclitaxel were dosed intraperitoneally twice a week for two consecutive weeks. Antibodies were injected at least 4 hours prior to paclitaxel injection.

In Vivo Anti-Tumor Effects on A2780ADR multidruQ Resistance Model.

A2780ADR cells were grown in RPMI-1640 supplemented with 10% FBS and 100 nM doxorubicin. Prior to implant cells were washed three times with D-PBS and resuspended in the same. Resuspended cells were mixed with an equal volume of Matrigel and xenografts were established by s.c. injection of 0.5×10⁶ tumor cells into 5-8-week-old female athymic nude mice. Tumors were measured three times a week using a calibrated digital caliper, and tumor volumes (TV) were calculated as per the formula: TV=/*L*S*S, where L is the long axis and s is the short axis of the tumor. When mean tumor volumes reached 100-200 mm³, mice were randomized into 10 treatment groups (n=8): (i) Isotype control hIgG1, 3 mg/kg, (ii) Isotype control hIgG1, 3 mg/kg plus paclitaxel, 20 mg/kg, (iii) KbisP1.1, 3 mg/kg, (iv) KbisP1.1, 3 mg/kg plus paclitaxel, 20 mg/kg, (v) 15D3 HIR2/KT14 KbR1/B1.89v1.huL1 (KV2) hmz LC, 3 mg/kg, (vi) 15D3 HIR2/KT14 KbR1/B1.89v1.huL1 (KV2) hmz LC, 3 mg/kg plus paclitaxel, 20 mg/kg, (vii) 15D3 HIR2/KT14 KbR1/B1.89v1.huL1 (KV2) hmz LC, 1 mg/kg, (viii) 15D3 HIR2/KT14 KbR1/B1.89v1.huL1 (KV2) hmz LC, 1 mg/kg plus paclitaxel, 20 mg/kg, (ix) 15D3 HIR2/KT14 KbR1/B1.89v1.huL1 (KV2) hmz LC, 0.3 mg/kg, (x) 15D3 HIR2/KT14 KbR1/B1.89v1.huL1 (KV2) hmz LC, 0.3 mg/kg plus paclitaxel, 20 mg/kg.

Antibody and paclitaxel (or vehicle) were administered i.p. two times per week for two weeks. Paclitaxel or vehicle was dosed four hours after the antibody.

### Flow Cytometry Assay

A flow cytometry-based assay was performed to determine 15D3 HIR2/KT14 KbR1/B1.89v1.huL1 (KV2) hmz LC bispecific antibody's ability to recruit effector immune cells to induce antibody-dependent cellular cytotoxicity (ADCC) against the human MES-SA/DX5 multidrug resistant cell model target. MES-SA/Dx5 cells were grown in RPMI-1640 supplemented with 10% FBS and 100 nM doxorubicin. Human PBMCs were isolated from leukocyte chambers (Stanford Blood Center) using SepMate-50 tubes (Stemcell) according to the manufacturer's protocol. Briefly, target cells were labeled with 5,6-Carboxyflurescein diacetate succinimidyl ester (CFSE) (Invitrogen) according to the manufacturer's protocol. CFSE labeled target cells were then treated with 5 and 20 μg/ml of either isotype antibody (human IgG1) or the 15D3 HIR2/KT14 KbR1/B1.89v1.huL1 (KV2) hmz LC bispecific antibody. For the dose-response study, target cells were treated with either media only or escalating concentrations of the bispecific antibody. PBMC effector cells were then added to the antibody-treated target cells at the indicated effector cell: target cell (E:T) ratios. About 4 hours after co-incubation of target and effector cells at 37° C., cells were washed twice with FACS buffer (PBS containing 2% FBS and 0.05% azide) and stained with Fixable Viability Dye eFluor 780 (FVD) (eBioscience) following the manufacturer's protocol. Cells were then washed and resuspend in FACS buffer and data acquired using an Attune NxT Flow Cytometer (Invitrogen). Data were analyzed using FlowJo software (BD). CFSE+ FVD+ population was defined as the percent of dead target cells. A two-way ANOVA followed by Tukey's HSD post hoc test was used to compare the isotype with the bispecific (15D3 HIR2/KT14 KbR1/B1.89v1.huL1 (KV2) hmz LC) treated groups. A value of p<0.05 was considered statistically significant. Statistical analyses were performed using GraphPad Prism application (San Diego, USA).

### Results

The 15D3 HIR2/KT14 KbR1/B1.89v1.huL1 (KV2) hmz LC bispecific antibody was efficacious against two multidrug resistant (MDR) cancer cell lines in xenograft studies. The A2780ADR MDR and the SA-MES-DX5 cancer cell lines express both CD47 and ABCB1.

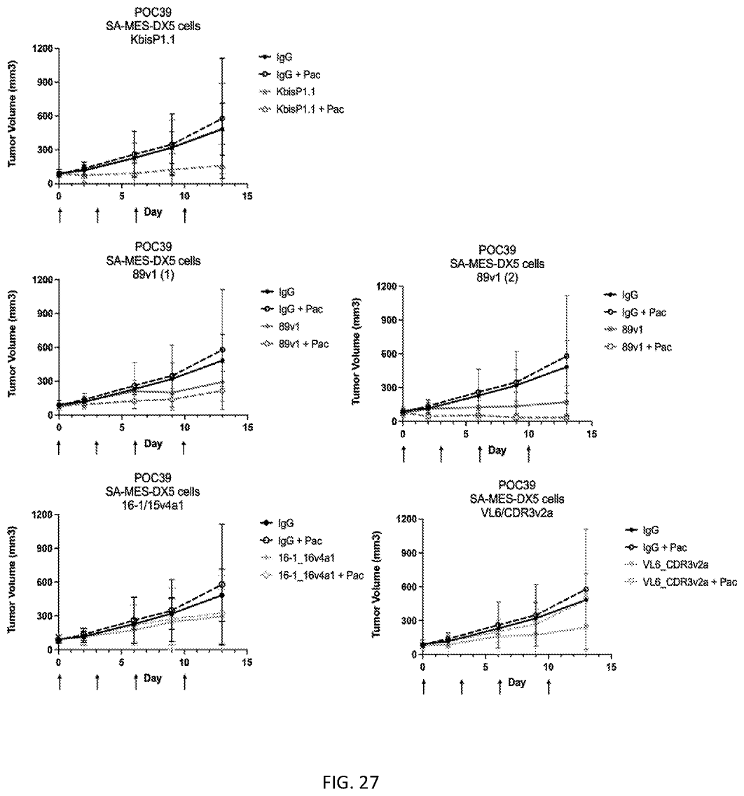

FIG. 27 presents results of assays testing the in vivo efficacy and dose-response of bispecific ABCB1XCD47 antibodies in the SA-MES-DX5 model. BisP1.1 and 15D3 HIR2/KT14 KbR1/B1.89v1.huL1 (KV2) hmz LC and 15D316-1 DD/KT14 KK/MRK16v4a1 hmz LC bispecific antibodies significantly inhibited tumor growth of SA-MES-DX5 cells both as a single agent and in combination with paclitaxel.

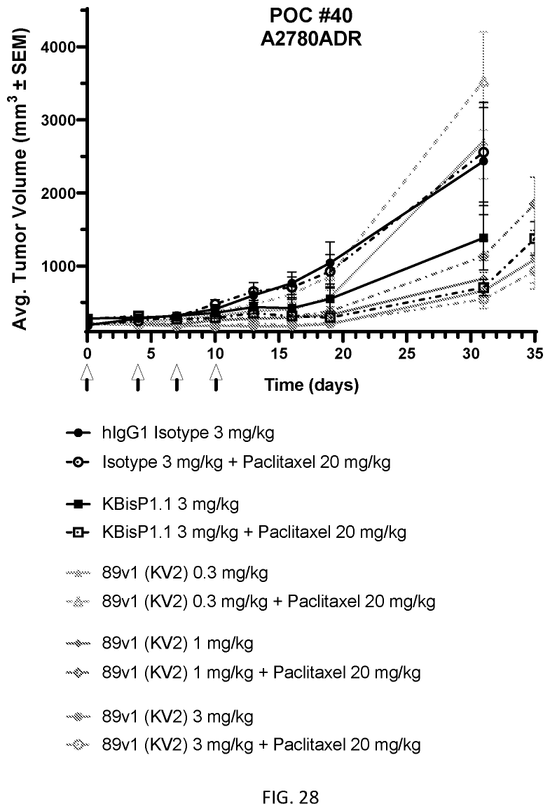

FIG. 28 presents results of assays testing the in vivo efficacy and dose-response of bispecific ABCB1XCD47 antibody 15D3 HIR2/KT14 KbR1/B1.89v1.huL1 (KV2) hmz LC in the A2780ADR model.

As shown in both in vivo models, 15D3 HIR2/KT14 KbR1/B1.89v1.huL1 (KV2) hmz LC bispecific antibody showed significant single agent activity that is possibly enhanced by paclitaxel-mediated cell-killing of multidrug resistant tumors in vivo. In particular, 15D3 HIR2/KT14 KbR1/B1.89v1.huL1 (KV2) hmz LC bispecific antibody showed significant dose-dependent single agent activity at 3 and 1 mg/ml doses.

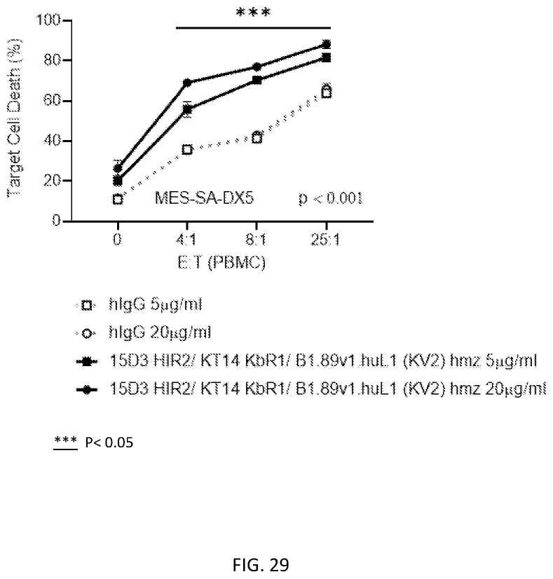

As shown in FIG. 29, the 15D3 HIR2/KT14 KbR1/B1.89v1.huL1 (KV2) hmz LC bispecific antibody induced significantly higher target cell death at all E:T ratios compared to the isotype control, showing that 15D3 HIR2/KT14 KbR1/B1.89v1.huL1 (KV2) hmz LC has the capacity to induce antibody-dependent cellular cytotoxicity. In addition, the tested bispecific antibody was able to induce ADCC in a dose-dependent manner at an E:T of 8:1, confirming the bispecific antibody's capacity to recruit immune effector cells to kill target cells.

### Example 8

### In Vitro Antibody Dependent Cell Cytotoxicity (ADCC) Assessment

A flow cytometry-based assay was performed to determine 15D3 HIR2/KT14 KbR1/B1.89v1.huL1 (KV2) hmz LC bispecific antibody's ability to recruit effector immune cells to induce antibody-dependent cellular cytotoxicity (ADCC) against the human MES-SA/DX5 multidrug resistant cell model target. MES-SA/Dx5 cells were grown in RPMI-1640 supplemented with 10% FBS and 100 nM doxorubicin. Human PBMCs were isolated from leukocyte chambers (Stanford Blood Center) using SepMate-50 tubes (Stemcell) according to the manufacturer's protocol. Briefly, target cells were labeled with 5,6-Carboxyflurescein diacetate succinimidyl ester (CFSE) (Invitrogen) according to the manufacturer's protocol. CFSE labeled target cells were then treated with 5 and 20 μg/ml of either isotype antibody (human IgG1) or the 15D3 HIR2/KT14 KbR1/B1.89v1.huL1 (KV2) hmz LC bispecific antibody. For the dose-response study, target cells were treated with either media only or escalating concentrations of the bispecific antibody. PBMC effector cells were then added to the antibody-treated target cells at the indicated effector cell: target cell (E:T) ratios. About 4 hours after co-incubation of target and effector cells at 37° C., cells were washed twice with FACS buffer (PBS containing 2% FBS and 0.05% azide) and stained with Fixable Viability Dye eFluor 780 (FVD) (eBioscience) following the manufacturer's protocol. Cells were then washed and resuspend in FACS buffer and data acquired using an Attune NxT Flow Cytometer (Invitrogen). Data were analyzed using FlowJo software (BD). CFSE+ FVD+ population was defined as the percent of dead target cells. A two-way ANOVA followed by Tukey's HSD post hoc test was used to compare the isotype with the bispecific (15D3 HIR2/KT14 KbR1/B1.89v1.huL1 (KV2) hmz LC) treated groups. A value of p<0.05 was considered statistically significant. Statistical analyses were performed using GraphPad Prism application (San Diego, USA).

### Results

As shown in FIG. 29, the 15D3 HIR2/KT14 KbR1/B1.89v1.huL1 (KV2) hmz LC bispecific antibody induced significantly higher target cell death at all E:T ratios compared to the isotype control, showing that 15D3 HIR2/KT14 KbR1/B1.89v1.huL1 (KV2) hmz LC has the capacity to induce antibody-dependent cellular cytotoxicity. In addition, the tested bispecific antibody was able to induce ADCC in a dose-dependent manner at an E:T of 8:1, confirming the bispecific antibody's capacity to recruit immune effector cells to kill target cells.

### Example 9

### Single Agent Activity and Enhancement of Paclitaxel-Mediated Cell Killing in A2780ADR Tumor Model In Vivo.

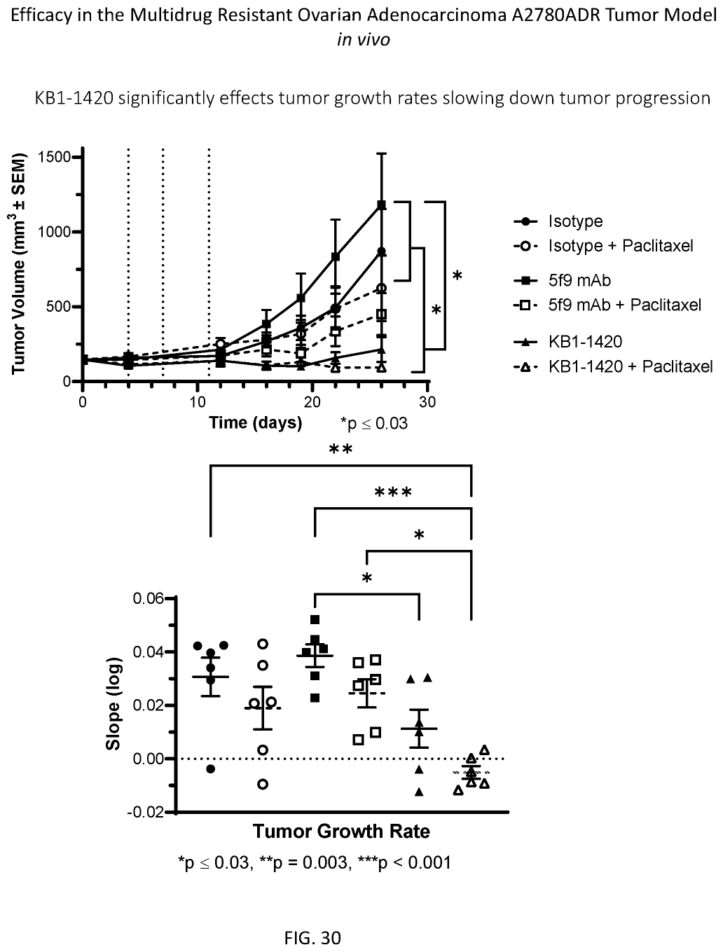

The A2780ADR multidrug resistant ovarian adenosarcoma tumor model is described in Example 7. The anti-CD47 antibody 5F9 used in this assay is in the IgG1 format. The results presented in FIG. 30 demonstrate that humanized bispecific ABCB1× CD47 antibody KB1-1420 significantly effects tumor growth rates, slowing down tumor progression in this in vivo model. Binding of K1-1420 to the ABCB1 antigen is essential to ensure single agent activity as well as to enhance paclitaxel-mediated cell killing.

### Example 10

### Therapeutic Effect Against Human Xenograft Tumors in Nude Mice

### Protocol

A2780ADR cells were grown in RPMI-1640 supplemented with 10% FBS and 100 nM doxorubicin. Prior to implant cells were washed three times with D-PBS and resuspended in the same. Resuspended cells were mixed with an equal volume of Matrigel and xenografts were established by s.c. injection of 0.5×106 tumor cells into 7-8-week-old female athymic nude mice (Charles River).

Tumors were measured three times a week using a calibrated digital caliper, and tumor volumes (TV) were calculated as per the formula: TV=/*L*S*S, where L is the long axis and s is the short axis of the tumor. When mean tumor volumes reached 100-200 mm³, mice were randomized into 6 treatment groups (n=6): (i) Isotype control hIgG1, 3 mg/kg, (ii) Isotype control hIgG1, 3 mg/kg plus paclitaxel, 20 mg/kg, (iii) 5f9 human IgG1 antibody, 3 mg/kg, (iv) 5f9 human IgG1 antibody, 3 mg/kg plus paclitaxel, 20 mg/kg, (v) 15D3 HIR2/KT14 KbR1/B1.89v1.huL1 (KV2) hmz LC, 3 mg/kg, (vi) 15D3 HIR2/KT14 KbR1/B1.89v1.huL1 (KV2) hmz LC, 3 mg/kg plus paclitaxel, 20 mg/kg.

Antibody and paclitaxel (or vehicle) were administered i.p. two time per week for two weeks. Antibodies were diluted in PBS to adjust for an injection volume of 5 ml/kg to deliver a 3 mg/kg dose. Paclitaxel or vehicle was dosed four hours after the antibody.

Tumor volumes and body weights were measured twice a week. Tumor measurements were taken using a calibrated digital electronic caliper. Tumor volume is calculated as per the formula TV=/*L*S*S where L is the long axis and S is the short axis of the tumor. Body weights were recorded before treatment started and were monitored throughout the study. Moribund animals predefined as rapid weight loss, loss of ability to ambulate, labored respiration, or inability to drink or feed to avoid were euthanized to reduce animal suffering.

Tumor volume and body weights were compared between different experimental groups using exact one-way analysis of variance (ANOVA). Kaplan-Meier survival curves were compared between different treatment groups using the log-rank (Mantel-Cox) test. Statistical analyses were performed using Graph Pad Prism (GraphPad, San Diego, CA, USA) software. The level of significance was set at p<0.05 for all analyses.

### Results

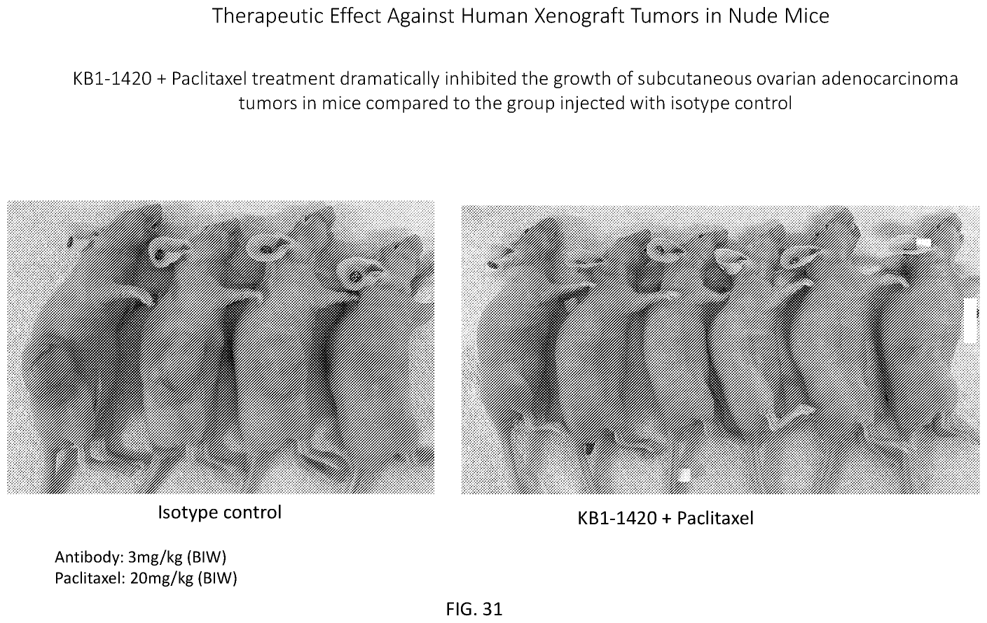

As demonstrated by the photos shown in FIG. 31, the four-dose regime of KB1-1420 shows strong therapeutic effect against human xenograft tumors in nude mice. In particular, the results show that KB1-1420+ paclitaxel treatment dramatically inhibited the growth of subcutaneous ovarian adenocarcinoma tumors in mice compared to the group injected with isotype control.

### Example 11

### In Vivo Activity in the Multidruq Resistant Uterine Sarcoma MES-SA/Dx5/ADR Tumor Model

### In Vivo Anti-Tumor Effects on MES-SA/Dx5/ADR Multidruq Resistance Model.

MES-SA/Dx5/ADR cells were grown in DMEM supplemented with 10% FBS and 1% penicillin and 1% streptomycin at 37° C., 5% CO₂ supplemented with 10% FBS and 100 nM doxorubicin. Prior to implant cells were washed three times with D-PBS and resuspended in the same. Resuspended cells were mixed with an equal volume of Matrigel and xenografts were established by s.c. injection of 0.5×10⁶ tumor cells into 7-8-week-old female athymic nude mice (Charles River).

Tumors were measured three times a week using a calibrated digital caliper, and tumor volumes (TV) were calculated as per the formula: TV=/2*L*S*S, where L is the long axis and s is the short axis of the tumor. When mean tumor volumes reached 100-200 mm³, mice were randomized into 6 treatment groups (n=7): (i) Isotype control hIgG1, 3 mg/kg, (ii) Isotype control hIgG1, 3 mg/kg plus paclitaxel, 20 mg/kg, (iii) 15D3 HIR2/KT14 KbR1/B1.89v1.huL1 (KV2) hmz LC, 3 mg/kg, (iv) 15D3 HIR2/KT14 KbR1/B1.89v1.huL1 (KV2) hmz LC, 3 mg/kg plus paclitaxel, 20 mg/kg, (v) 15D3 HIR2/KT14 KbR1/B1.89v1.huL1 (KV2) hmz LC, 3 mg/kg, (vi) 15D3 HIR2/KT14 KbR1/B1.89v1.huL1 (KV2) hmz LC, 3 mg/kg plus paclitaxel, 20 mg/kg, Antibody and paclitaxel (or vehicle) were administered i.p. two time per week for two weeks for the groups (i) to (iv) and two time per week for four weeks for the groups (v) and (vi). Antibodies were diluted in PBS to adjust for an injection volume of 5 ml/kg to deliver a 3 mg/kg dose. Paclitaxel or vehicle was dosed four hours after the antibody.

Measurements: Tumor volumes and body weights were measured twice a week. Tumor measurements were taken using a calibrated digital electronic caliper. Tumor volume is calculated as per the formula TV=/2*L*S*S where L is the long axis and S is the short axis of the tumor. Body weights were recorded before treatment started and were monitored throughout the study. Moribund animals predefined as rapid weight loss, loss of ability to ambulate, labored respiration, or inability to drink or feed to avoid were euthanized to reduce animal suffering.

Statistics: Tumor volume and body weights were compared between different experimental groups using exact one-way analysis of variance (ANOVA). Kaplan-Meier survival curves were compared between different treatment groups using the log-rank (Mantel-Cox) test. Statistical analyses were performed using Graph Pad Prism (GraphPad, San Diego, CA, USA) software. The level of significance was set at p<0.05 for all analyses.

### Results

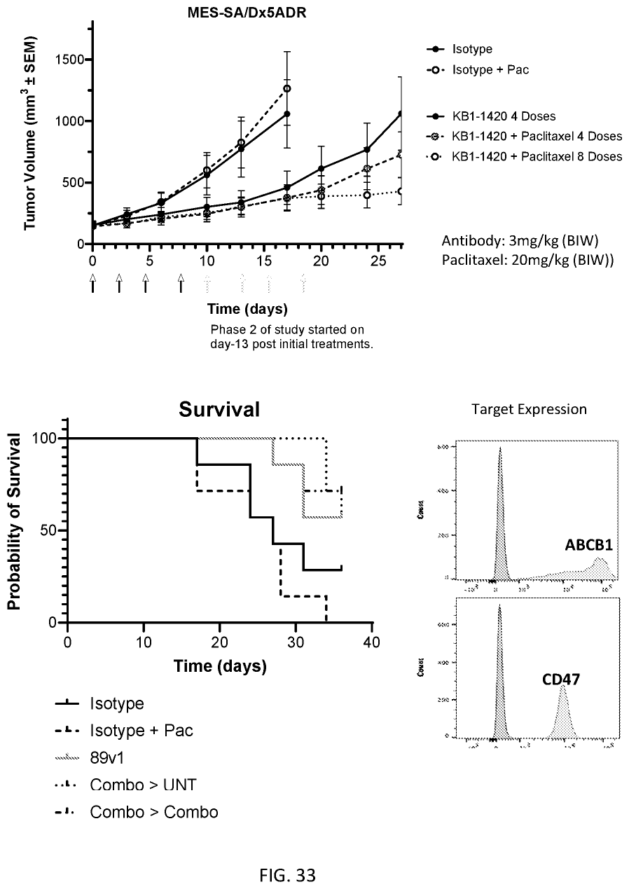

The results presented in FIG. 33 show that humanized bispecific ABCB1× CD47 antibody KB1-1420 is efficacious as a single agent and enhances paclitaxel-mediated cell killing in this model. KP1-1420, with and without paclitaxel, significantly enhances survival in a dose-dependent fashion.

### Example 12

### In Vivo Activity in the Multidruq Resistant Ovarian Adenocarcinoma A2780ADR Tumor Model

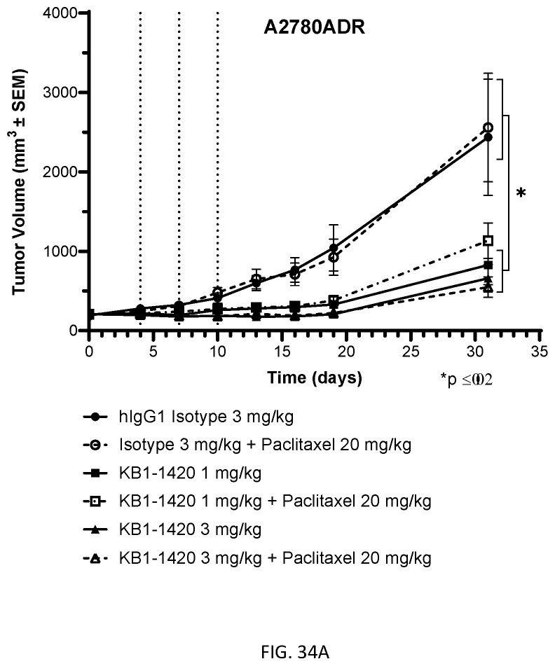

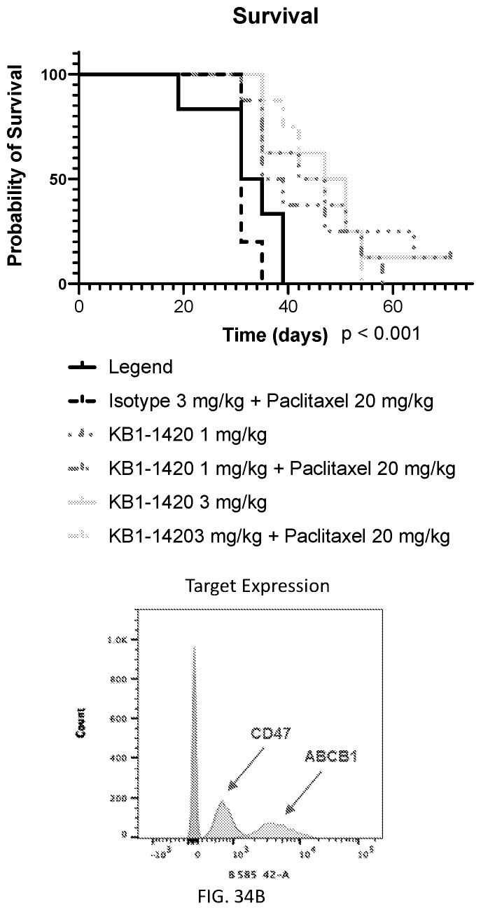

The tests were performed as described in Example 7. The data presented in FIG. 34 show that KB1-1420 is efficacious as a single agent and enhances paclitaxel-mediated cell killing in this multidrug resistant ovarian adenocarcinoma tumor model in vivo. As shown, KB1-1420 significantly improves survival in this A2780ADR multidrug resistant ovarian adenocarcinoma solid tumor xenograft model.

The antibodies tested in the foregoing examples are the result of combining the principles of using a common light chain with a robust new method for heterodimerization of the antibody heavy chains. The results presented demonstrate the efficacy of three different fully humanized bispecific antibodies (KB1-1418, KB1-1419 and KB1-1420) targeting ABCB1 and CD47 in a variety of in vitro tests and in different xenograft models of drug resistant tumors. Through manipulation of the light chains such antibodies can be engineered to have different desired characteristics. For example, as demonstrated by the receptor occupancy tests (see, e.g., FIG. 20), some antibodies may display stronger binding for ABCB1 and others for CD47, which may provide benefits in different clinical settings. The results obtained with the antibody KB1-1420 in xenograft models illustrate the efficacy of these bispecific antibodies in different solid tumors where both ABCB1 and CD47 are co-expressed at different levels and ratios as represented in the A2780ADR and the MES-SA/Dx5ADR tumor models (see, e.g., FIGS. 34A-34B and 33). The examples together illustrate the broad utility of such and similar bispecific antibodies in a wide range of clinical situations, when administered alone or in combination with other therapeutic approaches or agents.

1. A multi-specific IgG1 antibody molecule comprising a first heavy chain that comprises an antigen-binding site for multidrug resistance protein 1 (ABCB1) and a second heavy chain that comprises an antigen-binding site for a tumor associated antigen (TAA) each heavy chain comprising an Fc region, wherein one of the Fc regions comprises hole amino acid substitutions Y349C, T366S, L368A, and Y407V, in combination with amino acid substitution F405V or Q347R, wherein numbering is according to the EU numbering scheme.

2. The multi-specific IgG1 antibody molecule according to claim 1, wherein the other Fc region comprises knob amino acid substitutions S354C and T366W, wherein numbering is according to the EU numbering scheme.

3. The multi-specific IgG1 antibody molecule according to claim 1 or claim 2, wherein one of said Fc regions further comprises one or both of amino acid substitution D399K and E356K, wherein numbering is according to the EU numbering scheme.

4. The multi-specific IgG1 antibody molecule according to claim 3, wherein the other one of said Fc regions further comprises one or both of amino acid substitutions K409D and K392D

5. The multi-specific IgG1 antibody molecule according to any one of claims 1 to 4, which is bispecific.

6. The multi-specific IgG1 antibody molecule according to claim 5, which further comprises two identical light chain variable regions, each comprising an antigen-binding site for ABCB1, wherein the second heavy chain binds TAA when paired with one of the light chain variable regions.

7. The multi-specific IgG1 antibody molecule according to claim 6, wherein the light chain variable regions are humanized.

8. The multi-specific IgG1 antibody molecule according to claim 7, wherein the antigen-binding site of the two identical light chain variable regions comprises light chain CDRs 1-3 (LCDRs 1-3) of a light chain variable region sequence selected from the group consisting of:
(SEQ ID NO: 1)
DIVMTQTPLSSPVTLGQPASISCRSSQSIVHSTGNTYLEWYQQRPGQPPR
LLIYKISNRFSGVPDRFSGSGAGTDFTLKISRVEAEDVGVYYCFQASHFP
RTFGGGTKLEIKR
(MRK16 hmz);
(SEQ ID NO: 2)
DIVMTQSPLSLPVTPGEPASISCRSSQSLVHSNGNTYLEWYLQKPGQSPQ
LLIYKVSNRFSGVPDRFSGSGSGTDFTLKISRVEAEDVGVYYCFQGSHFP
RTFGQGTKLEIK
(B1VL6/CDR3v2a hmz);
(SEQ ID NO: 3)
DIVMTQTPLSSPVTLGQPASISCRSSQSIVHSTGNTYLEWYQQKPGQPPR
LLIYKISNRFSGVPDRFSGSGSGTDFTLKISRVEAEDVGVYYCFQASHFP
RTFGQGTKLEIK
(MRK16v4a1 hmz);
(SEQ ID NO: 4)
DIVMTQTPLSSPVTLGQPASISCRSSQSIVHSTGNTYLEWYQQKPGQPPR
LLIYKVSNRFSGVPDRFSGSGSGTDFTLKISRVEAEDVGVYYCFQASHFP
RTFGQGTKLEIK
(MRK16v4a12 hmz);
(SEQ ID NO: 6)
DIVMTQSPLSLPVTPGEPASISCRSSQTIVHSNGNTYLEWYLQKPGQSPQ
LLIYKVSKRFSGVPDRFSGSGSGTDFTLKISRVEAEDVGVYYCFQASHFP
RTFGQGTKLEIK
(B1.89v1.huL1 (KV2) hmz);
(SEQ ID NO: 7)
DVVLTQSPLSLPVTPGEPASISCRSSQNIVHSTGNTYLDWYLQKPGQSPQ
LLIYKVSNRFSGVPDRFSGSGSGTDFTLKISRVEAEDVGVYYCFQGSHIP
RTFGQGTKLEIKR
(B2.28.huL2);
and
(SEQ ID NO: 8)
DIVMTQSPLSLPVSLGDPASISCRSSQSLVHSNGNTYLEYYLQKPGQSPK
LLIYKVSNRFSGVPDRFSGSGSGTDFTLKISRVEAEDLGVYYCFQGSHFP
RTFGGGTKLEIK
(B1VL6/CDR3v2 hmz),
wherein LCDRs 1-3 are shown in bold.

9. The multi-specific IgG1 antibody molecule of claim 8, wherein the two identical light chain variable region sequences comprise a sequence selected from the group consisting of:
(SEQ ID NO: 1)
DIVMTQTPLSSPVTLGQPASISCRSSQSIVHSTGNTYLEWYQQRPGQPPR
LLIYKISNRFSGVPDRFSGSGAGTDFTLKISRVEAEDVGVYYCFQASHFP
RTFGGGTKLEIKR
(MRK16 hmz);
(SEQ ID NO: 2)
DIVMTQSPLSLPVTPGEPASISCRSSQSLVHSNGNTYLEWYLQKPGQSPQ
LLIYKVSNRFSGVPDRFSGSGSGTDFTLKISRVEAEDVGVYYCFQGSHFP
RTFGQGTKLEIK
(B1VL6/CDR3v2a hmz);
(SEQ ID NO: 3)
DIVMTQTPLSSPVTLGQPASISCRSSQSIVHSTGNTYLEWYQQKPGQPPR
LLIYKISNRFSGVPDRFSGSGSGTDFTLKISRVEAEDVGVYYCFQASHFP
RTFGQGTKLEIK
(MRK16v4a1 hmz);
(SEQ ID NO: 4)
DIVMTQTPLSSPVTLGQPASISCRSSQSIVHSTGNTYLEWYQQKPGQPPR
LLIYKVSNRFSGVPDRFSGSGSGTDFTLKISRVEAEDVGVYYCFQASHFP
RTFGQGTKLEIK
(MRK16v4a12 hmz);
(SEQ ID NO: 6)
DIVMTQSPLSLPVTPGEPASISCRSSQTIVHSNGNTYLEWYLQKPGQSPQ
LLIYKVSKRFSGVPDRFSGSGSGTDFTLKISRVEAEDVGVYYCFQASHFP
RTFGQGTKLEIK
(B1.89v1.huL1 (KV2) hmz);
(SEQ ID NO: 7)
DVVLTQSPLSLPVTPGEPASISCRSSQNIVHSTGNTYLDWYLQKPGQSPQ
LLIYKVSNRFSGVPDRFSGSGSGTDFTLKISRVEAEDVGVYYCFQGSHIP
RTFGQGTKLEIKR
(B2.28.huL2);
and
(SEQ ID NO: 8)
DIVMTQSPLSLPVSLGDPASISCRSSQSLVHSNGNTYLEYYLQKPGQSPK
LLIYKVSNRFSGVPDRFSGSGSGTDFTLKISRVEAEDLGVYYCFQGSHFP
RTFGGGTKLEIK
(B1VL6/CDR3v2 hmz).

10. The multi-specific IgG1 antibody molecule according to claim 9, wherein the antigen-binding site of the two identical light chain variable regions comprises light chain CDRs 1-3 (LCDRs 1-3) of a light chain variable region sequence selected from the group consisting of:
(SEQ ID NO: 6)
DIVMTQSPLSLPVTPGEPASISCRSSQTIVHSNGNTYLEWYLQKPGQSPQ
LLIYKVSKRFSGVPDRFSGSGSGTDFTLKISRVEAEDVGVYYCFQASHFP
RTFGQGTKLEIK
(B1.89v1.huL1 (KV2) hmz);
(SEQ ID NO: 7)
DVVLTQSPLSLPVTPGEPASISCRSSQNIVHSTGNTYLDWYLQKPGQSPQ
LLIYKVSNRFSGVPDRFSGSGSGTDFTLKISRVEAEDVGVYYCFQGSHIP
RTFGQGTKLEIKR
(B2.28.huL2);
and
(SEQ ID NO: 8)
DIVMTQSPLSLPVSLGDPASISCRSSQSLVHSNGNTYLEYYLQKPGQSPK
LLIYKVSNRFSGVPDRFSGSGSGTDFTLKISRVEAEDLGVYYCFQGSHFP
RTFGGGTKLEIK

11. The multi-specific IgG1 antibody molecule of claim 10, wherein the two identical light chain variable region sequences comprise the sequence of:
(SEQ ID NO: 6)
DIVMTQSPLSLPVTPGEPASISCRSSQTIVHSNGNTYLEWYLQKPGQSPQ
LLIYKVSKRFSGVPDRFSGSGSGTDFTLKISRVEAEDVGVYYCFQASHFP
RTFGQGTKLEIK
(B1.89v1.huL1 (KV2) hmz);
(SEQ ID NO: 7)
DVVLTQSPLSLPVTPGEPASISCRSSQNIVHSTGNTYLDWYLQKPGQSPQ
LLIYKVSNRFSGVPDRFSGSGSGTDFTLKISRVEAEDVGVYYCFQGSHIP
RTFGQGTKLEIKR
(B2.28.huL2);
or
(SEQ ID NO: 8)
DIVMTQSPLSLPVSLGDPASISCRSSQSLVHSNGNTYLEYYLQKPGQSPK
LLIYKVSNRFSGVPDRFSGSGSGTDFTLKISRVEAEDLGVYYCFQGSHFP
RTFGGGTKLEIK
(B1VL6/CDR3v2 hmz).

12. The multi-specific IgG1 antibody molecule according to any one of claims 1 to 11, wherein the first heavy chain is humanized.

13. The multi-specific IgG1 antibody molecule according to claim 12, wherein the antigen-binding site of the first heavy chain variable region comprises heavy chain CDRs 1-3 (H1CDRs 1-3) of a heavy chain variable region sequence selected from the group consisting of:
(SEQ ID NO: 9)
EVQLVESGGVVVQPGGSLRLSCAASGFTFSRYTMSWVRQAPGKGLEWVAT
ISSGGGNTYYPDSVKGRFTVSRDNSKNSLYLQMNSLRTEDTALYYCARYG
AGDAWFAYWGQGTLVTVSS
(15D3 hmz);
and
(SEQ ID NO: 10)
EVQLVESGGVVVQPGGSLRLSCAASGFTFSRYTMSWVRQAPGKGLEWVAT
ISSGGGNTYYPDSVKGRFTVSRDNSKNSLYLQMNSLRTEDTALYYCARYY
RYDAWFAYWGQGTLVTVSS
(15D3hmzv16-1 hG1),
where H1CDRs1-3 are shown in bold.

14. The multi-specific IgG1 antibody molecule according to claim 13, wherein the first heavy chain variable region sequence comprises:
(SEQ ID NO: 9)
EVQLVESGGVVVQPGGSLRLSCAASGFTFSRYTMSWVRQAPGKGLEWVAT
ISSGGGNTYYPDSVKGRFTVSRDNSKNSLYLQMNSLRTEDTALYYCARYG
AGDAWFAYWGQGTLVTVSS
(15D3 hmz);
or
(SEQ ID NO: 10)
EVQLVESGGVVVQPGGSLRLSCAASGFTFSRYTMSWVRQAPGKGLEWVAT
ISSGGGNTYYPDSVKGRFTVSRDNSKNSLYLQMNSLRTEDTALYYCARYY
RYDAWFAYWGQGTLVTVSS
(15D3hmzv16-1 hG1).

15. The multi-specific IgG1 antibody molecule according to any one of claims 1 to 14, wherein the TAA is selected from the group consisting of CD47, PD-L1, and EGFR.

16. The multi-specific IgG1 antibody molecule according to claim 15, wherein the TAA is CD47.

17. The multi-specific IgG1 antibody molecule according to claim 16, wherein the antigen-binding site of the second heavy chain variable region comprises CDRs1-3 (H2CDRs1-3) of heavy chain variable region sequence:
(SEQ ID NO: 11)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSS,
wherein H2CDRs1-3 are shown in bold.

18. The multi-specific IgG1 antibody molecule according to claim 17, wherein the second heavy chain variable region comprises the sequence:
(SEQ ID NO: 11)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSS.

19. The multi-specific IgG1 antibody molecule according to any one of claims 1 to 18, which antibody molecule exhibits at least one of decreased mispairing, decreased head-to-tail formation, decreased half-antibody production and increased overall yield of production as compared to an IgG1 antibody without Fc mutations or having only amino acid substitutions T366S, L368A, and Y407V in the first Fc region and only amino acid substitutions T366W in the second Fc region, wherein numbering is according to the EU numbering scheme.

20. A bispecific humanized IgG1 antibody molecule that binds multidrug resistance protein 1 (ABCB1) and a tumor associated antigen (TAA), the antibody molecule comprising two identical light chain variable regions, a first heavy chain variable region, and a second heavy chain variable region,
wherein the light chain variable regions each comprise an antigen-binding site for ABCB1, the first heavy chain variable region comprises an antigen-binding site for ABCB1, the second heavy chain variable region comprises an antigen-binding site for the TAA, and the second VH chain binds the TAA when paired with one of the VL chains, and
wherein the antigen-binding site of the two identical light chain variable regions comprises light chain CDRs 1-3 (LCDRs 1-3) of a light chain variable region sequence selected from the group consisting of:
(SEQ ID NO: 1)
DIVMTQTPLSSPVTLGQPASISCRSSQSIVHSTGNTYLEWYQQRPGQPPR
LLIYKISNRFSGVPDRFSGSGAGTDFTLKISRVEAEDVGVYYCFQASHFP
RTFGGGTKLEIKR
(MRK16 hmz);
(SEQ ID NO: 2)
DIVMTQSPLSLPVTPGEPASISCRSSQSLVHSNGNTYLEWYLQKPGQSPQ
LLIYKVSNRFSGVPDRFSGSGSGTDFTLKISRVEAEDVGVYYCFQGSHFP
RTFGQGTKLEIK
(B1VL6/CDR3v2a hmz);
(SEQ ID NO: 3)
DIVMTQTPLSSPVTLGQPASISCRSSQSIVHSTGNTYLEWYQQKPGQPPR
LLIYKISNRFSGVPDRFSGSGSGTDFTLKISRVEAEDVGVYYCFQASHFP
RTFGQGTKLEIK
(MRK16v4a1 hmz);
(SEQ ID NO: 4)
DIVMTQTPLSSPVTLGQPASISCRSSQSIVHSTGNTYLEWYQQKPGQPPR
LLIYKVSNRFSGVPDRFSGSGSGTDFTLKISRVEAEDVGVYYCFQASHFP
RTFGQGTKLEIK
(MRK16v4a12 hmz);
(SEQ ID NO: 6)
DIVMTQSPLSLPVTPGEPASISCRSSQTIVHSNGNTYLEWYLQKPGQSPQ
LLIYKVSKRFSGVPDRFSGSGSGTDFTLKISRVEAEDVGVYYCFQASHFP
RTFGQGTKLEIK
(B1.89v1.huL1 (KV2) hmz);
(SEQ ID NO: 7)
DVVLTQSPLSLPVTPGEPASISCRSSQNIVHSTGNTYLDWYLQKPGQSPQ
LLIYKVSNRFSGVPDRFSGSGSGTDFTLKISRVEAEDVGVYYCFQGSHIP
RTFGQGTKLEIKR
(B2.28.huL2);
and
(SEQ ID NO: 8)
DIVMTQSPLSLPVSLGDPASISCRSSQSLVHSNGNTYLEYYLQKPGQSPK
LLIYKVSNRFSGVPDRFSGSGSGTDFTLKISRVEAEDLGVYYCFQGSHFP
RTFGGGTKLEIK
(B1VL6/CDR3v2 hmz), wherein LCDRs 1-3 are shown in bold.

21. The bispecific humanized antibody molecule according to claim 20, wherein the two identical light chain variable region sequences comprise a sequence selected from the group consisting of:
(SEQ ID NO: 1)
DIVMTQTPLSSPVTLGQPASISCRSSQSIVHSTGNTYLEWYQQRPGQPPR
LLIYKISNRFSGVPDRFSGSGAGTDFTLKISRVEAEDVGVYYCFQASHFP
RTFGGGTKLEIKR
(MRK16 hmz);
(SEQ ID NO: 2)
DIVMTQSPLSLPVTPGEPASISCRSSQSLVHSNGNTYLEWYLQKPGQSPQ
LLIYKVSNRFSGVPDRFSGSGSGTDFTLKISRVEAEDVGVYYCFQGSHFP
RTFGQGTKLEIK
(B1VL6/CDR3v2a hmz);
(SEQ ID NO: 3)
DIVMTQTPLSSPVTLGQPASISCRSSQSIVHSTGNTYLEWYQQKPGQPPR
LLIYKISNRFSGVPDRFSGSGSGTDFTLKISRVEAEDVGVYYCFQASHFP
RTFGQGTKLEIK
(MRK16v4a1 hmz);
(SEQ ID NO: 4)
DIVMTQTPLSSPVTLGQPASISCRSSQSIVHSTGNTYLEWYQQKPGQPPR
LLIYKVSNRFSGVPDRFSGSGSGTDFTLKISRVEAEDVGVYYCFQASHFP
RTFGQGTKLEIK
(MRK16v4a12 hmz);
(SEQ ID NO: 6)
DIVMTQSPLSLPVTPGEPASISCRSSQTIVHSNGNTYLEWYLQKPGQSPQ
LLIYKVSKRFSGVPDRFSGSGSGTDFTLKISRVEAEDVGVYYCFQASHFP
RTFGQGTKLEIK
(B1.89v1.huL1 (KV2) hmz);
(SEQ ID NO: 7)
DVVLTQSPLSLPVTPGEPASISCRSSQNIVHSTGNTYLDWYLQKPGQSPQ
LLIYKVSNRFSGVPDRFSGSGSGTDFTLKISRVEAEDVGVYYCFQGSHIP
RTFGQGTKLEIKR
(B2.28.huL2);
and
(SEQ ID NO: 8)
DIVMTQSPLSLPVSLGDPASISCRSSQSLVHSNGNTYLEYYLQKPGQSPK
LLIYKVSNRFSGVPDRFSGSGSGTDFTLKISRVEAEDLGVYYCFQGSHFP
RTFGGGTKLEIK
(B1VL6/CDR3v2 hmz).

22. The bispecific humanized IgG1 antibody molecule according to claim 20, wherein the antigen-binding site of the two identical light chain variable regions comprises light chain CDRs 1-3 (LCDRs 1-3) of a light chain variable region sequence selected from the group consisting of:
(SEQ ID NO: 6)
DIVMTQSPLSLPVTPGEPASISCRSSQTIVHSNGNTYLEWYLQKPGQSPQ
LLIYKVSKRFSGVPDRFSGSGSGTDFTLKISRVEAEDVGVYYCFQASHFP
RTFGQGTKLEIK
(B1.89v1.huL1 (KV2) hmz);
(SEQ ID NO: 7)
DVVLTQSPLSLPVTPGEPASISCRSSQNIVHSTGNTYLDWYLQKPGQSPQ
LLIYKVSNRFSGVPDRFSGSGSGTDFTLKISRVEAEDVGVYYCFQGSHIP
RTFGQGTKLEIKR
(B2.28.huL2);
and
(SEQ ID NO: 8)
DIVMTQSPLSLPVSLGDPASISCRSSQSLVHSNGNTYLEYYLQKPGQSPK
LLIYKVSNRFSGVPDRFSGSGSGTDFTLKISRVEAEDLGVYYCFQGSHFP
RTFGGGTKLEIK
(B1VL6/CDR3v2 hmz), wherein LCDRs 1-3 are shown in bold.

23. The bispecific humanized IgG1 antibody molecule according to claim 21, wherein the two identical light chain variable region sequences comprise the sequence of:
(B1.89v1.huL1 (KV2) hmz)
(SEQ ID NO: 6)
DIVMTQSPLSLPVTPGEPASISCRSSQTIVHSNGNTYLEWYLQKPGQS
PQLLIYKVSKRFSGVPDRFSGSGSGTDFTLKISRVEAEDVGVYYCFQA
SHFPRTFGQGTKLEIK;
(B2.28.huL2)
(SEQ ID NO: 7)
DVVLTQSPLSLPVTPGEPASISCRSSQNIVHSTGNTYLDWYLQKPGQS
PQLLIYKVSNRFSGVPDRFSGSGSGTDFTLKISRVEAEDVGVYYCFQG
SHIPRTFGQGTKLEIKR;
or
(B1VL6/CDR3v2 hmz)
(SEQ ID NO: 8)
DIVMTQSPLSLPVSLGDPASISCRSSQSLVHSNGNTYLEYYLQKPGQS
PKLLIYKVSNRFSGVPDRFSGSGSGTDFTLKISRVEAEDLGVYYCFQG
SHFPRTFGGGTKLEIK.

24. The bispecific humanized IgG1 antibody molecule according to any one of claims 20 to 23, wherein the antigen-binding site of the first heavy chain variable region comprises heavy chain CDRs 1-3 (H1CDRs 1-3) of a heavy chain variable region sequence selected from the group consisting of:
(15D3 hmz)
(SEQ ID NO: 9)
EVQLVESGGVVVQPGGSLRLSCAASGFTFSRYTMSWVRQAPGKGLEWV
ATISSGGGNTYYPDSVKGRFTVSRDNSKNSLYLQMNSLRTEDTALYYC
ARYGAGDAWFAYWGQGTLVTVSS;
and
(15D3hmzv16-1 hG1)
(SEQ ID NO: 10)
EVQLVESGGVVVQPGGSLRLSCAASGFTFSRYTMSWVRQAPGKGLEWV
ATISSGGGNTYYPDSVKGRFTVSRDNSKNSLYLQMNSLRTEDTALYYC
ARYYRYDAWFAYWGQGTLVTVSS,
where H1CDRs1-3 are shown in bold.

25. The bispecific humanized IgG1 antibody molecule according to claim 24, wherein the first heavy chain variable region sequence comprises:
(15D3 hmz)
(SEQ ID NO: 9)
EVQLVESGGVVVQPGGSLRLSCAASGFTFSRYTMSWVRQAPGKGLEWV
ATISSGGGNTYYPDSVKGRFTVSRDNSKNSLYLQMNSLRTEDTALYYC
ARYGAGDAWFAYWGQGTLVTVSS;
or
(15D3hmzv16-1 hG1)
(SEQ ID NO: 10)
EVQLVESGGVVVQPGGSLRLSCAASGFTFSRYTMSWVRQAPGKGLEWV
ATISSGGGNTYYPDSVKGRFTVSRDNSKNSLYLQMNSLRTEDTALYYC
ARYYRYDAWFAYWGQGTLVTVSS.

26. The bispecific humanized IgG1 antibody molecule according to any one of claims 20 to 25, wherein the TAA is selected from the group consisting of CD47, PD-L1, and EGFR.

27. The bispecific humanized IgG1 antibody molecule according to claim 26, wherein the TAA is CD47.

28. The bispecific humanized IgG1 antibody molecule according to claim 27, wherein the antigen-binding site of the second heavy chain variable region comprises CDRs1-3 (H2CDRs1-3) of heavy chain variable region sequence:
(SEQ ID NO: 11)
QVQLVQSGAEVKKPGASVKVSCKASGYTFTNYNMHWVRQAPGQRLEWM
GTIYPGNDDTSYNQKFKDRVTITADTSASTAYMELSSLRSEDTAVYYC
ARGGYRAMDYWGQGTLVTVSS.

29. The bispecific humanized IgG1 antibody molecule according to claim 28, wherein the second heavy chain variable region comprises the sequence:
(SEQ ID NO: 11)
QVQLVQSGAEVKKPGASVKVSCKASGYTFTNYNMHWVRQAPGQRLEWM
GTIYPGNDDTSYNQKFKDRVTITADTSASTAYMELSSLRSEDTAVYYC
ARGGYRAMDYWGQGTLVTVSS.

30. The bispecific humanized IgG1 antibody molecule according to any one of claims 20 to 29, wherein the first and the second heavy chains each comprise an Fc region, and one of the Fc regions comprises hole amino acid substitutions Y349C, T366S, L368A, and Y407V, wherein the numbering is according to the EU numbering scheme.

31. The bispecific humanized IgG1 antibody molecule according to 30 wherein said Fc region further comprises amino acid substitution F405V or Q347R.

32. The bispecific humanized IgG1 antibody molecule according to claim 30 or claim 31, wherein the other Fc region comprises knob amino acid substitutions S354C and T366W, wherein numbering is according to the EU numbering scheme.

33. The bispecific humanized IgG1 antibody molecule according any one of claims 30 to 32, wherein one of said Fc regions further comprises one or both of amino acid substitutions D399K and E356K, wherein numbering is according to the EU numbering scheme.

34. The bispecific humanized IgG1 antibody molecule according to claim 33, wherein the other Fc region further comprises one or both or amino acid substitutions K409D and K392D, wherein numbering is according to the EU numbering scheme.

35. The bispecific humanized IgG1 antibody molecule according to any one of claims 20 to 34, wherein one of the Fc regions comprises a set of amino acid substitutions and amino acid residues selected from the group consisting of:
(a) Y349C, T366S, L368A, Y407V, E356, E357, F405V;
(b) Y349C, T366S, L368A, Y407V, E356, E357, F405V, K409D;
(c) Y349C, T366S, L368A, Y407V, E356, E357, K409D;
(d) Y349C, T366S, L368A, Y407V, E356, E357, D399K;
(e) Y349C, T366S, L368A, Y407V, E356, E357, F405V, D399K; and
(f) Y349C, T366S, L368A, Y407V, E356, E357, Q347R,
wherein numbering is according to the EU numbering scheme.

36. The bispecific humanized IgG1 antibody molecule according to claim 35, wherein the other Fc region comprises a set of amino acid substitutions and amino acid residues selected from the group consisting of:
(a) S354C, T366W, E356, E357;
(b) S354C, T366W, E356, E357, D399K;
(c) S354C, T366W, E356, E357, K409D;
(d) S354C, T366W, E356, E357, K360E,
wherein numbering is according to the EU numbering scheme.

37. The bispecific humanized IgG1 antibody molecule according to claim 36, wherein one of the Fc regions comprises the set of amino acid substitutions and amino acid residues Y349C, T366S, L368A, Y407V, E356, E357, F405V, and the other Fc region comprises the set of amino acid substitutions and amino acid residues S354C, T366W, E356, E357, wherein numbering is according to the EU numbering scheme.

38. The bispecific humanized IgG1 antibody molecule according to claim 36, wherein one of the Fc regions comprises the set of amino acid substitutions and amino acid residues Y349C, T366S, L368A, Y407V, E356, E357, K409D, and the other Fc region comprises the set of amino acid substitutions and amino acid residues S354C, T366W, E356, E357, D399K, wherein numbering is according to the EU numbering scheme.

39. The bispecific humanized IgG1 antibody molecule according to claim 36, wherein one of the Fc regions comprises the set of amino acid substitutions and amino acid residues Y349C, T366S, L368A, Y407V, E356, E357, F405V, D399K, and the other Fc region comprises the set of amino acid substitutions and amino acid residues S354C, T366W, E356, E357, K409D, wherein numbering is according to the EU numbering scheme.

40. The bispecific humanized IgG1 antibody molecule according to claim 36, wherein one of the Fc regions comprises the set of amino acid substitutions and amino acid residues Y349C, T366S, L368A, Y407V, E356, E357, Q347R, and the other Fc region comprises the set of amino acid substitutions and amino acid residues S354C, T366W, E356, E357, K360E, wherein numbering is according to the EU numbering scheme.

41. The bispecific humanized IgG1 antibody molecule according to claim 36, wherein the first heavy chain comprises an Fc region of the following sequence:
HC1 (hG1-HIR2):
(SEQ ID NO: 12)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
RVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVCTLPPSREE
MTKNQVSLSCAVKGFYPSDIAVEWESNGQPENNYKTTPPVLDSDGSFV
LVSKLTVDKSRWQQGNVFSCSVMHEALHNHYTQKSLSLSPGK
and the second heavy chain comprises an Fc region of the following sequence:
HC1 (hG1-KbR1):
(SEQ ID NO: 22)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
RVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVYTLPPCREE
MTKNQVSLWCLVKGFYPSDIAVEWESNGQPENNYKTTPPVLDSDGSFF
LYSKLTVDKSRWQQGNVFSCSVMHEALHNHYTQKSLSLSPGK
wherein the amino acid residues facilitating pairing are shown in bold.

42. The bispecific humanized IgG1 antibody molecule according to claim 36, wherein the first heavy chain comprises an Fc region of the following sequence:
HC1 (hG1-HIR4):
(SEQ ID NO: 16)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
RVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVCTLPPSREE
MTKNQVSLSCAVKGFYPSDIAVEWESNGQPENNYKTTPPVLDSDGSFF
LVSDLTVDKSRWQQGNVFSCSVMHEALHNHYTQKSLSLSPGK
and the second heavy chain comprises an Fc region of the following sequence:
HC2 (hG1-KbR3):
(SEQ ID NO: 23)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
RVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVYTLPPCREE
MTKNQVSLWCLVKGFYPSDIAVEWESNGQPENNYKTTPPVLKSDGSFF
LYSKLTVDKSRWQQGNVFSCSVMHEALHNHYTQKSLSLSPGK
wherein the amino acid residues facilitating pairing are shown in bold.

43. The bispecific humanized IgG1 antibody molecule according to claim 36, wherein the first heavy chain comprises an Fc region of the following sequence:
HC1 (hG1-HIR6):
(SEQ ID NO: 18)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
RVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVCTLPPSREE
MTKNQVSLSCAVKGFYPSDIAVEWESNGQPENNYKTTPPVLKSDGSFV
LVSKLTVDKSRWQQGNVFSCSVMHEALHNHYTQKSLSLSPGK
and the second heavy chain comprises an Fc region of the following sequence:
HC2 (hG1-KbR5):
(SEQ ID NO: 24)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
RVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVYTLPPCREE
MTKNQVSLWCLVKGFYPSDIAVEWESNGQPENNYKTTPPVLDSDGSFF
LYSDLTVDKSRWQQGNVFSCSVMHEALHNHYTQKSLSLSPGK
wherein the amino acid residues facilitating pairing are shown in bold.

44. The bispecific humanized IgG1 antibody molecule according to claim 36, wherein the first heavy chain comprises an Fc region of the following sequence:
HC1 (hG1-HIR7):
(SEQ ID NO: 19)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
RVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPRVCTLPPSREE
MTKNQVSLSCAVKGFYPSDIAVEWESNGQPENNYKTTPPVLDSDGSFF
LVSKLTVDKSRWQQGNVFSCSVMHEALHNHYTQKSLSLSPGK
and the second heavy chain comprises an Fc region of the following sequence:
HC2 (hG1-KbR7):
(SEQ ID NO: 25)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
RVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVYTLPPCREE
MTENQVSLWCLVKGFYPSDIAVEWESNGQPENNYKTTPPVLDSDGSFF
LYSKLTVDKSRWQQGNVFSCSVMHEALHNHYTQKSLSLSPGK
wherein the amino acid residues facilitating pairing are shown in bold.

45. The bispecific humanized IgG1 antibody molecule according to any one of claims 41 to 44, which antibody molecule exhibits at least one of decreased mispairing, decreased head-to-tail formation, decreased half-antibody production and increased overall yield of production as compared to an IgG1 antibody without Fc mutations or having only amino acid substitutions T366S, L368A, and Y407V in the first Fc region and only amino acid substitutions T366W in the second Fc region, wherein numbering is according to the EU numbering scheme.

46. A humanized antibody light chain binding to ABCB1 comprising CDRs 1-3 (LCDRs 1-3) of a light chain variable region sequence selected from the group consisting of:
(MRK16 hmz)
(SEQ ID NO: 1)
DIVMTQTPLSSPVTLGQPASISCRSSQSIVHSTGNTYLEWYQQRPGQP
PRLLIYKISNRFSGVPDRFSGSGAGTDFTLKISRVEAEDVGVYYCFQA
SHFPRTFGGGTKLEIKR;
(B1VL6/CDR3v2a hmz)
(SEQ ID NO: 2)
DIVMTQSPLSLPVTPGEPASISCRSSQSLVHSNGNTYLEWYLQKPGQS
PQLLIYKVSNRFSGVPDRFSGSGSGTDFTLKISRVEAEDVGVYYCFQG
SHFPRTFGQGTKLEIK;
(MRK16v4a1 hmz)
(SEQ ID NO: 3)
DIVMTQTPLSSPVTLGQPASISCRSSQSIVHSTGNTYLEWYQQKPGQP
PRLLIYKISNRFSGVPDRFSGSGSGTDFTLKISRVEAEDVGVYYCFQA
SHFPRTFGQGTKLEIK;
(MRK16v4a12 hmz)
(SEQ ID NO: 4)
DIVMTQTPLSSPVTLGQPASISCRSSQSIVHSTGNTYLEWYQQKPGQP
PRLLIYKVSNRFSGVPDRFSGSGSGTDFTLKISRVEAEDVGVYYCFQA
SHFPRTFGQGTKLEIK;
(B1.89v1.huL 1 (KV2) hmz)
(SEQ ID NO: 6)
DIVMTQSPLSLPVTPGEPASISCRSSQTIVHSNGNTYLEWYLQKPGQS
PQLLIYKVSKRFSGVPDRFSGSGSGTDFTLKISRVEAEDVGVYYCFQA
SHFPRTFGQGTKLEIK;
(B2.28.huL2)
(SEQ ID NO: 7)
DVVLTQSPLSLPVTPGEPASISCRSSQNIVHSTGNTYLDWYLQKPGQS
PQLLIYKVSNRFSGVPDRFSGSGSGTDFTLKISRVEAEDVGVYYCFQG
SHIPRTFGQGTKLEIKR;
and
(B1VL6/CDR3v2 hmz)
(SEQ ID NO: 8)
DIVMTQSPLSLPVSLGDPASISCRSSQSLVHSNGNTYLEYYLQKPGQS
PKLLIYKVSNRFSGVPDRFSGSGSGTDFTLKISRVEAEDLGVYYCFQG
SHFPRTFGGGTKLEIK,
wherein LCDRs 1-3 are shown in bold.

47. The humanized antibody light chain according to claim 46 comprising a sequence selected from the group consisting of:
(MRK16 hmz)
(SEQ ID NO: 1)
DIVMTQTPLSSPVTLGQPASISCRSSQSIVHSTGNTYLEWYQQRPGQP
PRLLIYKISNRFSGVPDRFSGSGAGTDFTLKISRVEAEDVGVYYCFQA
SHFPRTFGGGTKLEIKR;
(B1VL6/CDR3v2a hmz)
(SEQ ID NO: 2)
DIVMTQSPLSLPVTPGEPASISCRSSQSLVHSNGNTYLEWYLQKPGQS
PQLLIYKVSNRFSGVPDRFSGSGSGTDFTLKISRVEAEDVGVYYCFQG
SHFPRTFGQGTKLEIK;
(MRK16v4a1 hmz)
(SEQ ID NO: 3)
DIVMTQTPLSSPVTLGQPASISCRSSQSIVHSTGNTYLEWYQQKPGQP
PRLLIYKISNRFSGVPDRFSGSGSGTDFTLKISRVEAEDVGVYYCFQA
SHFPRTFGQGTKLEIK;
(MRK16v4a12 hmz)
(SEQ ID NO: 4)
DIVMTQTPLSSPVTLGQPASISCRSSQSIVHSTGNTYLEWYQQKPGQP
PRLLIYKVSNRFSGVPDRFSGSGSGTDFTLKISRVEAEDVGVYYCFQA
SHFPRTFGQGTKLEIK;
(B1.89v1.huL1 (KV2) hmz)
(SEQ ID NO: 6)
DIVMTQSPLSLPVTPGEPASISCRSSQTIVHSNGNTYLEWYLQKPGQS
PQLLIYKVSKRFSGVPDRFSGSGSGTDFTLKISRVEAEDVGVYYCFQA
SHFPRTFGQGTKLEIK;
(B2.28.huL2)
(SEQ ID NO: 7)
DVVLTQSPLSLPVTPGEPASISCRSSQNIVHSTGNTYLDWYLQKPGQS
PQLLIYKVSNRFSGVPDRFSGSGSGTDFTLKISRVEAEDVGVYYCFQG
SHIPRTFGQGTKLEIKR;
and
(B1VL6/CDR3v2 hmz)
(SEQ ID NO: 8)
DIVMTQSPLSLPVSLGDPASISCRSSQSLVHSNGNTYLEYYLQKPGQS
PKLLIYKVSNRFSGVPDRFSGSGSGTDFTLKISRVEAEDLGVYYCFQG
SHFPRTFGGGTKLEIK.

48. A humanized anti-ABCB1 antibody comprising the humanized light chain of claim 46 or 47.

49. The humanized anti-ABCB1 antibody of claim 48, which is multi-specific.

50. The humanized anti-ABCB1 antibody of claim 49, which is bispecific.

51. The humanized anti-ABCB1 antibody of claim 50, which binds to a tumor-associated antigen (TAA).

52. The humanized anti-ABCB1 antibody of claim 50, wherein the TAA is CD47, PD-L1 or EGFR.

53. A composition comprising a multi-specific IgG1 antibody molecule according to any one of claims 1 to 19, a bispecific humanized IgG1 antibody molecule according to any one of claims 20 to 45, or the humanized anti-ABCB1 antibody according to any one of claims 48 to 52, in combination and a carrier.

54. The composition of claim 53, which is a pharmaceutical composition.

55. Use of a multi-specific IgG1 antibody molecule according to any one of claims 1 to 19, a bispecific humanized IgG1 antibody molecule according to any one of claims 20 to 45, or the humanized anti-ABCB1 antibody according to any one of claims 48 to 52, in the preparation of a medicament for the treatment of cancer in a patient.

56. A multi-specific IgG1 antibody molecule according to any one of claims 1 to 19, a bispecific humanized IgG1 antibody molecule according to any one of claims 20 to 45, or the humanized anti-ABCB1 antibody according to any one of claims 48-52, for use in a method of treating cancer in a patient in need.

57. A method of treating cancer comprising administering to a patient in need a therapeutically effective amount of a multi-specific IgG1 antibody molecule according to any one of claims 1 to 19, a bispecific humanized IgG1 antibody molecule according to any one of claims 20 to 45, or the humanized anti-ABCB1 antibody according to any one of claims 48 to 52.

58. The method according to claim 57, wherein the method comprises administering the multi-specific IgG1 antibody molecule or the bispecific humanized IgG1 antibody molecule in combination with at least one additional active agent, wherein the at least one additional active agent comprises a chemotherapeutic agent, an inhibitor of a multidrug resistance transporter, an immunotherapy agent, or a combination thereof.

59. The method according to claim 58, wherein the at least one additional active agent is a chemotherapeutic agent.

60. The method according to claim 59, wherein the chemotherapeutic agent is a taxol, a vinca alkaloid, or an anthracycline.

61. The method according to claim 58, wherein the subject has been treated previously for the cancer.

62. The method according to claim 61, wherein the cancer is drug resistant or multidrug resistant.

63. The method according to claim 62, wherein the cancer is resistant to a chemotherapeutic agent.

64. The method according to claim 62, wherein the cancer is resistant to an immunotherapy agent.

65. The method according to claim 62, wherein the cancer is resistant to an inhibitor of a multidrug resistance transporter.

66. The method according to any one of claims 62 to 65, further comprising administering at least one additional active agent to the subject.

67. The method according to claim 66, wherein the at least one additional active agent comprises a chemotherapy agent.

68. The method according to claim 67, wherein the chemotherapy agent is a taxol, a vinca alkaloid, or an anthracycline.

69. The method according to claim 66, wherein the at least one additional active agent comprises an inhibitor of a multidrug resistance transporter.

70. The method according to claim 66, wherein the at least one additional active agent comprises an immunotherapy agent.

71. The method according to claim 70, wherein the immunotherapy agent comprises an antibody or a modified immune cell.

72. The method according to any one of claims 57 to 71, wherein the method increases the effectiveness of the at least one additional active agent as compared to treatment with the at least one additional active agent alone.

73. The method according to claim 72, wherein the increased effectiveness comprises an at least 5% increase in cancer cell killing.

74. One or more nucleic acids comprising one or more sequences encoding a multi-specific IgG1 antibody molecule according to any one of claims 1 to 19, a bispecific humanized IgG1 antibody molecule according to any one of claims 20 to 45, or the humanized anti-ABCB1 antibody according to any one of claims 48 to 52.

75. The one or more nucleic acid according to claim 74 operably linked to a promoter.

76. One or more recombinant expression vectors comprising the one of more nucleic acids according to claim 74 or 75.

77. A mammalian cell genetically modified with the one or more recombinant expression vectors according to claim 76.

78. The mammalian cell according to claim 77, which is an immune cell.

79. A kit comprising a multi-specific IgG1 antibody molecule according to any one of claims 1 to 19, a bispecific humanized IgG1 antibody molecule according to any one of claims 20 to 45, or the humanized anti-ABCB1 antibody according to any one of claims 48 to 52, a nucleic acid according to claim 74 or 75, or a mammalian cell according to claim 77 or claim 78.

80. The kit according to claim 79, further comprising at least one additional active agent.

81. The kit according to claim 80, wherein the addition active agent comprises a chemotherapy agent.

82. The kit according to any one of claims 79 to 81, further comprising instructions to use.


SEQUENCE LISTING (121 sequences)

SEQ ID NO: 1 - protein, 113 aa, synthetic construct
DIVMTQTPLSSPVTLGQPASISCRSSQSIVHSTGNTYLEWYQQRPGQPPRLLIYKISNRFSGVPDRFSGSGAGTDFTLKISRVEAEDVGVYYCFQASHFPRTFGGGTKLEIKR

SEQ ID NO: 2 - protein, 112 aa, synthetic construct
DIVMTQSPLSLPVTPGEPASISCRSSQSLVHSNGNTYLEWYLQKPGQSPQLLIYKVSNRFSGVPDRFSGSGSGTDFTLKISRVEAEDVGVYYCFQGSHFPRTFGQGTKLEIK

SEQ ID NO: 3 - protein, 112 aa, synthetic construct
DIVMTQTPLSSPVTLGQPASISCRSSQSIVHSTGNTYLEWYQQKPGQPPRLLIYKISNRFSGVPDRFSGSGSGTDFTLKISRVEAEDVGVYYCFQASHFPRTFGQGTKLEIK

SEQ ID NO: 4 - protein, 112 aa, synthetic construct
DIVMTQTPLSSPVTLGQPASISCRSSQSIVHSTGNTYLEWYQQKPGQPPRLLIYKVSNRFSGVPDRFSGSGSGTDFTLKISRVEAEDVGVYYCFQASHFPRTFGQGTKLEIK

SEQ ID NO: 5 - skipped

SEQ ID NO: 6 - protein, 112 aa, synthetic construct
DIVMTQSPLSLPVTPGEPASISCRSSQTIVHSNGNTYLEWYLQKPGQSPQLLIYKVSKRFSGVPDRFSGSGSGTDFTLKISRVEAEDVGVYYCFQASHFPRTFGQGTKLEIK

SEQ ID NO: 7 - protein, 113 aa, synthetic construct
DVVLTQSPLSLPVTPGEPASISCRSSQNIVHSTGNTYLDWYLQKPGQSPQLLIYKVSNRFSGVPDRFSGSGSGTDFTLKISRVEAEDVGVYYCFQGSHIPRTFGQGTKLEIKR

SEQ ID NO: 8 - protein, 112 aa, synthetic construct
DIVMTQSPLSLPVSLGDPASISCRSSQSLVHSNGNTYLEYYLQKPGQSPKLLIYKVSNRFSGVPDRFSGSGSGTDFTLKISRVEAEDLGVYYCFQGSHFPRTFGGGTKLEIK

SEQ ID NO: 9 - protein, 119 aa, synthetic construct
EVQLVESGGVVVQPGGSLRLSCAASGFTFSRYTMSWVRQAPGKGLEWVATISSGGGNTYYPDSVKGRFTVSRDNSKNSLYLQMNSLRTEDTALYYCARYGAGDAWFAYWGQGTLVTVSS

SEQ ID NO: 10 - protein, 119 aa, synthetic construct
EVQLVESGGVVVQPGGSLRLSCAASGFTFSRYTMSWVRQAPGKGLEWVATISSGGGNTYYPDSVKGRFTVSRDNSKNSLYLQMNSLRTEDTALYYCARYYRYDAWFAYWGQGTLVTVSS

SEQ ID NO: 11 - protein, 117 aa, synthetic construct
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 12 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFVLVSKLTVDKSRWQQGNVFSCSVMHEALHNHYTQKSLSLSPGK

SEQ ID NO: 13 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 14 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 15 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFVLVSDLTVDKSRWQQGNVFSCSVMHEALHNHYTQKSLSLSPGK

SEQ ID NO: 16 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSDLTVDKSRWQQGNVFSCSVMHEALHNHYTQKSLSLSPGK

SEQ ID NO: 17 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLKSDGSFFLVSKLTVDKSRWQQGNVFSCSVMHEALHNHYTQKSLSLSPGK

SEQ ID NO: 18 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLKSDGSFVLVSKLTVDKSRWQQGNVFSCSVMHEALHNHYTQKSLSLSPGK

SEQ ID NO: 19 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 20 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 21 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 22 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLKSDGSFFLYSKLTVDKSRWQQGNVFSCSVMHEALHNHYTQKSLSLSPGK

SEQ ID NO: 24 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSDLTVDKSRWQQGNVFSCSVMHEALHNHYTQKSLSLSPGK

SEQ ID NO: 25 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTENQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 27 - protein, 112 aa, synthetic construct
DVLMTQTPVSLSVSLGDQASISCRSSQSIVHSTGNTYLEWYLQKPGQSPKLLIYKISNRFSGVPDRFSGSGSGTDFTLKISRVEAEDLGVYYCFQASHFPRTFGGGTKLEIK

SEQ ID NO: 28 - protein, 119 aa, synthetic construct
EVKVVESGGVLVRPGGSLKLSCAASGFTFSRYTMSWVRQTPEKRLEWVATISSGGGNTYYPDSVKGRFTVSRDNAMSSLYLQMSSLRSEDTALYYCARYGAGDAWFAYWGQGTLVTVSS

SEQ ID NO: 29 - skipped

SEQ ID NO: 30 - protein, 448 aa, synthetic construct
EVKVVESGGVLVRPGGSLKLSCAASGFTFSRYTMSWVRQTPEKRLEWVATISSGGGNTYYPDSVKGRFTVSRDNAMSSLYLQMSSLRSEDTALYYCARYGAGDAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 31 - protein, 448 aa, synthetic construct
EVQLVESGGVVVQPGGSLRLSCAASGFTFSRYTMSWVRQAPGKGLEWVATISSGGGNTYYPDSVKGRFTVSRDNSKNSLYLQMNSLRTEDTALYYCARYGAGDAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 32 - protein, 446 aa, synthetic construct
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 33 - protein, 448 aa, synthetic construct
EVQLVESGGVVVQPGGSLRLSCAASGFTFSRYTMSWVRQAPGKGLEWVATISSGGGNTYYPDSVKGRFTVSRDNSKNSLYLQMNSLRTEDTALYYCARYGAGDAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFVLVSKLTVDKSRWQQGNVFSCSVMHEALHNHYTQKSLSLSPG

SEQ ID NO: 34 - protein, 448 aa, synthetic construct
EVQLVESGGVVVQPGGSLRLSCAASGFTFSRYTMSWVRQAPGKGLEWVATISSGGGNTYYPDSVKGRFTVSRDNSKNSLYLQMNSLRTEDTALYYCARYGAGDAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSDLTVDKSRWQQGNVFSCSVMHEALHNHYTQKSLSLSPG

SEQ ID NO: 35 - protein, 447 aa, synthetic construct
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 447 aa, synthetic construct
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFVLVSDLTVDKSRWQQGNVFSCSVMHEALHNHYTQKSLSLSPGK

SEQ ID NO: 37 - protein, 219 aa, synthetic construct
DIVMTQSPLSLPVTPGEPASISCRSSQTIVHSNGNTYLEWYLQKPGQSPQLLIYKVSKRFSGVPDRFSGSGSGTDFTLKISRVEAEDVGVYYCFQASHFPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 219 aa, synthetic construct
DVLMTQTPVSLSVSLGDQASISCRSSQSIVHSTGNTYLEWYLQKPGQSPKLLIYKISNRFSGVPDRFSGSGSGTDFTLKISRVEAEDLGVYYCFQASHFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 219 aa, synthetic construct
DIVMTQSPLSLPVTPGEPASISCRSSQSLVHSNGNTYLEWYLQKPGQSPQLLIYKVSNRFSGVPDRFSGSGSGTDFTLKISRVEAEDVGVYYCFQGSHFPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 15 aa, synthetic construct
  VARIANT 6..15: note = Wherein 1 or 2 copies of GGGGS can be deleted
GGGGSGGGGSGGGGS

SEQ ID NO: 41 - protein, 17 aa, synthetic construct
  VARIANT 8..17: note = Wherein 1 or 2 copies of GGGGS can be deleted
GSTGGGSGGGGSGGGGS

SEQ ID NO: 42 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 123 aa, synthetic construct
EVQLVESGGVVVQPGGSLRLSCAASGFTFSRYTMSWVRQAPGKGLEWVATISSGGGNTYYPDSVKGRFTVSRDNSKNSLYLQMNSLRTEDTALYYCARYGRGDAWFAYWGQGTLVTVSSASTK

SEQ ID NO: 44 - protein, 123 aa, synthetic construct
EVQLVESGGVVVQPGGSLRLSCAASGFTFSRYTMSWVRQAPGKGLEWVATISSGGGNTYYPDSVKGRFTVSRDNSKNSLYLQMNSLRTEDTALYYCARYYAYDAWFAYWGQGTLVTVSSASTK

SEQ ID NO: 45 - protein, 123 aa, synthetic construct
EVQLVESGGVVVQPGGSLRLSCAASGFTFSRYTMSWVRQAPGKGLEWVATISSGGGNTYYPDSVKGRFTVSRDNSKNSLYLQMNSLRTEDTALYYCARYGAGDAWFASWGQGTLVTVSSASTK

SEQ ID NO: 46 - protein, 123 aa, synthetic construct
EVQLVESGGVVVQPGGSLRLSCAASGFTFSRYTMSWVRQAPGKGLEWVATISSGGGNTYYPDSVKGRFTVSRDNSKNSLYLQMNSLRTEDTALYYCARYYAYDAWFASWGQGTLVTVSSASTK

SEQ ID NO: 47 - protein, 119 aa, synthetic construct
EVKVVESGGVLVRPGGSLKLSCAASGFTFSRYTMSWVRQTPEKRLEWVATISSGGGNTYYPDSVKGRFTVSRDNAMSSLYLQMSSLRSEDTALYYCARYGAGDAWFAYWGQGTLVTVSA

SEQ ID NO: 48 - protein, 112 aa, synthetic construct
DVLMTQTPLSLPVSLGDQASISCRSSQSIVHSTGNTYLEWYLQKPGQSPKLLIYKVSNRFSGVPDRFSGSGSGTDFTLKISRLEAEDLGVYYCFQGSHFPRTFGGGTRLEIK

SEQ ID NO: 49 - protein, 112 aa, synthetic construct
DVLMTQTPLSLPVSLGDQASISCRSSQSIVHRTGNTYLEWYLQKPGQSPKLLIYKVSNRFSGVPDRFSGSGSGTDFTLKISRVEAEDLGVYYCFQGSHVPYTFGGGTKLEIK

SEQ ID NO: 50 - protein, 120 aa, synthetic construct
EVKLVESGGGLVKFGGSLKLSCAASGFTLSSYYMSWVRQSPEKRLELVAVINSNGGSTYYPDTVKGRFTISRDNAKNTLYLQMSSLKSEDTALYYCARPFYYSNSPFAYWGQGTLVTVSS

SEQ ID NO: 51 - protein, 108 aa, synthetic construct
EIVLTQSPATLSLSPGERATLSCRASQSVGGSYLAWYQQKPGQAPRLLIYGASRRATGIPARFSGSGSGTDFTLTISSLQPEDFASYFCQQTNTFPLTFGGGTKVEIK

SEQ ID NO: 52 - protein, 114 aa, synthetic construct
QVQLVQSGGGVVQPGRSLRLSCAASGFTFSSYGMHWVRQDPGKGLMWVSSISTDGSATKYADSVKGRFTISRDNAKNTVSLQMNSLRAEDTAVYYCVGGFLGWWGQGTLVTVSS

SEQ ID NO: 53 - protein, 118 aa, synthetic construct [sequence omitted]

SEQ ID NO: 54 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 451 aa, synthetic construct
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 121 aa, synthetic construct [sequence omitted]

SEQ ID NO: 58 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 59 - protein, 120 aa, synthetic construct [sequence omitted]

SEQ ID NO: 60 - protein, 119 aa, synthetic construct [sequence omitted]

SEQ ID NO: 61 - protein, 118 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVS

SEQ ID NO: 62 - protein, 120 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 63 - protein, 121 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 64 - protein, 121 aa, synthetic construct
EVQLVESGGGLVQPGGSLRLSCAASGFSLSGSWIHWVRQAPGKGLEWVGWINPYRGYAYYADSVKGRFTISADTSKNTAYLQMNSLRAEDTAVYYCAREYSGWGGSSVGYAMDYWGQGTLV

SEQ ID NO: 65 - protein, 116 aa, synthetic construct
EVQLLESGGGLVQPGGSLRLSCAASGFTFSSYAMNWVRQAPGKGLEWVSTTSGSGASTYYADSVKGRFTISRDNSKNTLYLQMNSLRAEDTAVYYCAKIWIAFDIWGQGTMVTVSS

SEQ ID NO: 66 - protein, 120 aa, synthetic construct
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSS

SEQ ID NO: 67 - protein, 119 aa, synthetic construct
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 68 - protein, 120 aa, synthetic construct
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 69 - protein, 117 aa, synthetic construct
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 70 - protein, 113 aa, synthetic construct
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSS

SEQ ID NO: 71 - protein, 118 aa, synthetic construct
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 72 - protein, 119 aa, synthetic construct [sequence omitted]

SEQ ID NO: 73 - protein, 118 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 74 - protein, 119 aa, synthetic construct
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 75 - protein, 115 aa, synthetic construct [sequence omitted]

SEQ ID NO: 76 - protein, 121 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 77 - protein, 120 aa, synthetic construct
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSS

SEQ ID NO: 78 - skipped

SEQ ID NO: 79 - protein, 121 aa, synthetic construct
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSS

SEQ ID NO: 80 - protein, 124 aa, synthetic construct
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 81 - protein, 122 aa, synthetic construct [sequence omitted]

SEQ ID NO: 82 - protein, 121 aa, synthetic construct
QVQLQQSGAEVKKPGSSVKVSCKASGYTFTSYNMHWVKQAPGQGLEWIGAIYPGMGDTSYNQKFKGKATLTADESTNTAYMELSSLRSEDTAFYYCARSTYYGGDWYFDVWGQGTTVTVSS

SEQ ID NO: 83 - skipped

SEQ ID NO: 84 - protein, 122 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 85 - protein, 122 aa, synthetic construct [sequence omitted]

SEQ ID NO: 86 - protein, 121 aa, synthetic construct [sequence omitted]

SEQ ID NO: 87 - protein, 122 aa, synthetic construct
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSG

SEQ ID NO: 88 - skipped

SEQ ID NO: 89 - protein, 118 aa, synthetic construct
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSS

SEQ ID NO: 90 - protein, 116 aa, synthetic construct [sequence omitted]

SEQ ID NO: 91 - protein, 116 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 92 - protein, 119 aa, synthetic construct
QVQLVESGGGVVQPGRSLRLSCAASGFTFSSYDMHWVRQAPGKGLEWVAVIWYDGSNKYYADSVKGRFTISRDNSKNTLYLQMNSLRAEDTAVYYCARGSGNWGFFDYWGQGTLVTVSS

SEQ ID NO: 93 - protein, 117 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 94 - protein, 117 aa, synthetic construct
QVQLVQSGAEVKKPGASVKVSCKVSGYTFTDYYITWVRQAPGQALEWMGWIYPGSGNTKYSQKFQGRFVFSVDTSASTAYLQISSLKAEDTAVYYCANYGNYWFAYWGQGTLVTVSS

SEQ ID NO: 95 - protein, 117 aa, synthetic construct
QVQLQQSAAELARPGASVKMSCKASGYTFTDLSMHWIKQRPGQGLEWIGYITPSSGYTAYNQKFKGKTTLTADKSSSTAYMQLSSLTSEDSAVYYCARLILQRGAYWGQGTLVTVSA

SEQ ID NO: 96 - protein, 124 aa, synthetic construct
EVQLVESGGGLVQPGGSLRLSCATSGFDFSRYWMSWVRQAPGKGLVWIGEVNPDSTSINYTPSLKDQFTISRDNAKNTLYLQMNSLRAEDTAVYYCTRPNYYGSRYHYYAMDYWGQGTLVTVSS

SEQ ID NO: 97 - protein, 121 aa, synthetic construct
QLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSS

SEQ ID NO: 98 - protein, 122 aa, synthetic construct
EVQLVESGGGLVQPGGSLRLSCAASGFTFSVYYMNWVRQAPGKGLEWVSDINNEGGTTYYADSVKGRFTISRDNSKNSLYLQMNSLRAEDTAVYYCARDAGYSNHVPIFDSWGQGTLVTVSS

SEQ ID NO: 99 - protein, 118 aa, synthetic construct
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 100 - protein, 117 aa, synthetic construct
EVQLLESGGGLVQPGGSLRLSCAASGFTFSSYAYSWVRQAPGKGLEWVSAISGSGGRTYYADSVKGRFTISRDNSKNTLYLQMNSLRAEDTAVYYCARLGYGRVDEWGRGTLVTVSS

SEQ ID NO: 101 - protein, 122 aa, synthetic construct
QVQLVQSGAEVEKPGASVKVSCKASGYTFTSYWITWVRQAPGQGLEWMGDIYPGSGNTNYNEKFKTRVTITADKSTSTAYMELSSLRSEDTAVYYCAKEGGLTTEDYALDYWGQGTLVTVSS

SEQ ID NO: 102 - protein, 117 aa, synthetic construct [sequence omitted]

SEQ ID NO: 103 - protein, 116 aa, synthetic construct
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSS

SEQ ID NO: 104 - skipped

SEQ ID NO: 105 - protein, 118 aa, synthetic construct [sequence omitted]

SEQ ID NO: 106 - protein, 125 aa, synthetic construct [sequence omitted]

SEQ ID NO: 107 - protein, 5 aa, synthetic construct
DSWIH

SEQ ID NO: 108 - protein, 17 aa, synthetic construct
WISPYGGSTYYADSVKG

SEQ ID NO: 109 - protein, 9 aa, synthetic construct
RHWPGGFDY

SEQ ID NO: 110 - protein, 7 aa, synthetic construct
SGDYYWS

SEQ ID NO: 111 - protein, 16 aa, synthetic construct
YIYYSGSTDYNPSLKS

SEQ ID NO: 112 - protein, 11 aa, synthetic construct
VSIFGVGTFDY

SEQ ID NO: 113 - protein, 5 aa, synthetic construct
NYGVH

SEQ ID NO: 114 - protein, 16 aa, synthetic construct
VIWSGGNTDYNTPFTS

SEQ ID NO: 115 - protein, 11 aa, synthetic construct
ALTYYDYEFAY

SEQ ID NO: 116 - protein, 1280 aa, synthetic construct
MDLEGDRNGGAKKKNFFKLNNKSEKDKKEKKPTVSVFSMFRYSNWLDKLYMVVGTLAAIIHGAGLPLMMLVFGEMTDIFANAGNLEDLMSNITNRSDINDTGFFMNLEEDMTRYAYYYSGIGAGVLVAAYIQVSFWCLAAGRQIHKIRKQFFHAIMRQEIGWFDVHDVGELNTRLTDDVSKINEGIGDKIGMFFQSMATFFTGFIVGFTRGWKLTLVILAISPVLGLSAAVWAKILSSFTDKELLAYAKAGAVAEEVLAAIRTVIAFGGQKKELERYNKNLEEAKRIGIKKAITANISIGAAFLLIYASYALAFWYGTTLVLSGEYSIGQVLTVFFSVLIGAFSVGQASPSIEAFANARGAAYEIFKIIDNKPSIDSYSKSGHKPDNIKGNLEFRNVHFSYPSRKEVKILKGLNLKVQSGQTVALVGNSGCGKSTTVQLMQRLYDPTEGMVSVDGQDIRTINVRFLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQVALDKARKGRTTIVIAHRLSTVRNADVIAGFDDGVIVEKGNHDELMKEKGIYFKLVTMQTAGNEVELENAADESKSEIDALEMSSNDSRSSLIRKRSTRRSVRGSQAQDRKLSTKEALDESIPPVSFWRIMRLNLTEWPYFVVGVFCAIINGGLQPAFAIIFSKIIGVFTRIDDPETKRQNSNLFSLLFLALGIISFITFFLQGFTFGKAGEILTKRLRYMVFRSMLRQDVSWFDDPKNTTGALTTRLANDAAQVKGAIGSRLAVITQNIANLGTGIIISFIYGWQLTLLLLAIVPIIAIAGVVEMKMLSGQALKDKKELEGSGKIATEAIENFRTVVSLTQEQKFEHMYAQSLQVPYRNSLRKAHIFGITFSFTQAMMYFSYAGCFRFGAYLVAHKLMSFEDVLLVFSAVVFGAMAVGQVSSFAPDYAKAKISAAHIIMIIEKTPLIDSYSTEGLMPNTLEGNVTFGEVVFNYPTRPDIPVLQGLSLEVKKGQTLALVGSSGCGKSTVVQLLERFYDPLAGKVLLDGKEIKRLNVQWLRAHLGIVSQEPILFDCSIAENIAYGDNSRVVSQEEIVRAATEANIHAFIESLPNKYSTKVGDKGTQLSGGQKQRIAIARALVRQPHILLLDEATSALDTESEKVVQEALDKAREGRTCIVIAHRLSTIQNADLIVVFQNGRVKEHGTHQQLLAQKGIYFSMVSVQAGTKRQ

SEQ ID NO: 117 - protein, 111 aa, synthetic construct
DIVMTQSPLSLPVTPGEPASISCRSSQSIVYSNGNTYLGWYLQKPGQSPQLLIYKVSNRFSGVPDRFSGSGSGTDFTLKISRVEADVGVYYCFQGSHVPYTFGQGTKLEIK

SEQ ID NO: 118 - protein, 119 aa, synthetic construct
EVILVESGGGLVKPGGSLKLSCAASGFTFSSYTMSWVRQTPEKRLEWVATISSGGGNTYYPDSVKGRFTISRDNAKNNLYLQMSSLRSEDTALYYCARYYRYEAWFASWGQGTLVTVSA

SEQ ID NO: 119 - protein, 193 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSS

SEQ ID NO: 120 - protein, 446 aa, synthetic construct
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 446 aa, synthetic construct
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLKSDGSFFLYSKLTVDKSRWQQGNVFSCSVMHEALHNHYTQKSLSLSPG